SEQUENCE LISTING (74 sequences)

SEQ ID NO: 1 - RNA, 7 nt, Homo sapiens
  misc_feature (1)..(1): M is A or C
  misc_feature (6)..(7): R is A or G
maggurr

SEQ ID NO: 2 - RNA, 9 nt, Homo sapiens
acuuaccug

SEQ ID NO: 3 - RNA, 8 nt, Homo sapiens
auuugugg

SEQ ID NO: 4 - DNA, 153 bp, Artificial Sequence
  main U1 snRNA sequence (nt 410-562) of the U1_ 256 sequence
gcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttcccctg

SEQ ID NO: 5 - DNA, 7 bp, Unknown
  branch point consensus sequence in eukaryotes
  misc_feature (1)..(1): Y is a pyrimidine
  misc_feature (2)..(2): N is any nucleotide
  misc_feature (3)..(3): Y is a pyrimidine
  misc_feature (5)..(5): R is a purine
ynytrac

SEQ ID NO: 6 - RNA, 3 nt, Unknown
  3′ acceptor splice site consensus sequence in eukaryotes
  misc_feature (1)..(1): Y is a pyrimidine
yag

SEQ ID NO: 7 - DNA, 8 bp, Unknown
  canonical splice donor site consensus sequence
  misc_feature (2)..(3): cleavage site
  misc_feature (5)..(5): R is a purine
aggtragt

SEQ ID NO: 8 - DNA, 8 bp, Human immunodeficiency virus type 1
  misc_feature (2)..(3): cleavage site
  misc_feature (5)..(5): R is a purine
tggtragt

SEQ ID NO: 9 - DNA, 30 bp, Unknown
  splice donor region of the lentiviral vector genome
ggggcggcgactggtgagtacgccaaaaat

SEQ ID NO: 10 - DNA, 30 bp, Artificial Sequence
  MSD-2KO mutated splice donor region
ggggcggcgactgcagacaacgccaaaaat

SEQ ID NO: 11 - DNA, 30 bp, Artificial Sequence
  MSD-2KOv2 mutated splice donor region
ggggcggcgagtggagactacgccaaaaat

SEQ ID NO: 12 - DNA, 32 bp, Artificial Sequence
  MSD-2KOm5 mutated splice donor region
ggggaaggcaacagataaatatgccttaaaat

SEQ ID NO: 13 - DNA, 19 bp, Unknown
  splice donor region of lentiviral genome
ggcgactggtgagtacgcc

SEQ ID NO: 14 - DNA, 7 bp, Unknown
  major splice donor and cryptic splice donor region core sequence
  misc_feature (1)..(1): cleavage site
  misc_feature (4)..(5): cleavage site
gtgagta

SEQ ID NO: 15 - DNA, 5 bp, Artificial Sequence
  major splice donor consensus sequence
ctggt

SEQ ID NO: 16 - DNA, 5 bp, Artificial Sequence
  cryptic splice donor site consensus sequence
tgagt

SEQ ID NO: 17 - DNA, 6 bp, Artificial Sequence
  splice donor region
cagaca

SEQ ID NO: 18 - DNA, 19 bp, Artificial Sequence
  mutated splice donor region
ggcgactgcagacaacgcc

SEQ ID NO: 19 - DNA, 9 bp, Artificial Sequence
  mutated splice donor region
gtggagact

SEQ ID NO: 20 - DNA, 19 bp, Artificial Sequence
  mutated splice donor region
ggcgagtggagactacgcc

SEQ ID NO: 21 - DNA, 23 bp, Artificial Sequence
  mutated splice donor region
aaggcaacagataaatatgcctt

SEQ ID NO: 22 - DNA, 642 bp, Artificial Sequence
  DNA-based expression constructs for the modified U1 snRNAs
taaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgctgcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcccaagatctcatttgccgtgcgcgcttgcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttcccctggtttcaaaagtagactgtacgctaagggtcatatctttttttgttttggtttgtgtcttggttggcgtcttaaatgttaa

SEQ ID NO: 23 - DNA, 15 bp, Artificial Sequence
  U1_16 HIV-1 target sequence [NL4-3]
gaccagatctgagcc

SEQ ID NO: 24 - DNA, 17 bp, Artificial Sequence
  U1_16 U1 snRNA target-annealing sequence
atggctcagatctggtc

SEQ ID NO: 25 - DNA, 15 bp, Artificial Sequence
  U1_31 HIV-1 target sequence [NL4-3]
tgggagctctctggc

SEQ ID NO: 26 - DNA, 17 bp, Artificial Sequence
  U1_31 U1 snRNA target-annealing sequence
atgccagagagctccca

SEQ ID NO: 27 - DNA, 15 bp, Artificial Sequence
  U1_76 HIV-1 target sequence [NL4-3]
taaagcttgccttga

SEQ ID NO: 28 - DNA, 17 bp, Artificial Sequence
  U1_76 U1 snRNA target-annealing sequence
attcaaggcaagcttta

SEQ ID NO: 29 - DNA, 15 bp, Artificial Sequence
  U1_136 HIV-1 target sequence [NL4-3]
tagagatccctcaga

SEQ ID NO: 30 - DNA, 17 bp, Artificial Sequence
  U1_136 U1 snRNA target-annealing sequence
attctgagggatctcta

SEQ ID NO: 31 - DNA, 9 bp, Artificial Sequence
  U1_179(9nt) HIV-1 target sequence [NL4-3]
gcagtggcg

SEQ ID NO: 32 - DNA, 11 bp, Artificial Sequence
  U1_179(9nt) U1 snRNA target-annealing sequence
atcgccactgc

SEQ ID NO: 33 - DNA, 15 bp, Artificial Sequence
  U1_181 HIV-1 target sequence [NL4-3]
agtggcgcccgaaca

SEQ ID NO: 34 - DNA, 17 bp, Artificial Sequence
  U1_181 U1 snRNA target-annealing sequence
attgttcgggcgccact

SEQ ID NO: 35 - DNA, 15 bp, Artificial Sequence
  U1_196 HIV-1 target sequence [NL4-3]
gggacttgaaagcga

SEQ ID NO: 36 - DNA, 17 bp, Artificial Sequence
  U1_196 U1 snRNA target-annealing sequence
attcgctttcaagtccc

SEQ ID NO: 37 - DNA, 15 bp, Artificial Sequence
  U1_211 HIV-1 target sequence [NL4-3]
aagggaaaccagagg

SEQ ID NO: 38 - DNA, 17 bp, Artificial Sequence
  U1_211 U1 snRNA target-annealing sequence
atcctctggtttccctt

SEQ ID NO: 39 - DNA, 15 bp, Artificial Sequence
  U1_226 HIV-1 target sequence [NL4-3]
agctctctcgacgca

SEQ ID NO: 40 - DNA, 17 bp, Artificial Sequence
  U1_226 U1 snRNA target-annealing sequence
attgcgtcgagagagct

SEQ ID NO: 41 - DNA, 15 bp, Artificial Sequence
  U1_241 HIV-1 target sequence [NL4-3]
ggactcggcttgctg

SEQ ID NO: 42 - DNA, 17 bp, Artificial Sequence
  U1_241 U1 snRNA target-annealing sequence
atcagcaagccgagtcc

SEQ ID NO: 43 - DNA, 15 bp, Artificial Sequence
  U1_256 HIV-1 target sequence [NL4-3]
aagcgcgcacggcaa

SEQ ID NO: 44 - DNA, 17 bp, Artificial Sequence
  U1_256 U1 snRNA target-annealing sequence
atttgccgtgcgcgctt

SEQ ID NO: 45 - DNA, 15 bp, Artificial Sequence
  U1_271 HIV-1 target sequence [NL4-3]
gaggcgaggggcggc

SEQ ID NO: 46 - DNA, 17 bp, Artificial Sequence
  U1_271 U1 snRNA target-annealing sequence
atgccgcccctcgcctc

SEQ ID NO: 47 - DNA, 15 bp, Artificial Sequence
  U1_286 HIV-1 target sequence [NL4-3]
gactggtgagtacgc

SEQ ID NO: 48 - DNA, 17 bp, Artificial Sequence
  U1_286 U1 snRNA target-annealing sequence
atgcgtactcaccagtc

SEQ ID NO: 49 - DNA, 11 bp, Artificial Sequence
  U1_305(9nt) HIV-1 target sequence [NL4-3]
aattttgacta

SEQ ID NO: 50 - DNA, 11 bp, Artificial Sequence
  U1_305(9nt) U1 snRNA target-annealing sequence
atgtcaaaatt

SEQ ID NO: 51 - DNA, 15 bp, Artificial Sequence
  U1_305 HIV-1 target sequence [NL4-3]
aattttgactagcgg

SEQ ID NO: 52 - DNA, 17 bp, Artificial Sequence
  U1_305 U1 snRNA target-annealing sequence
atccgctagtcaaaatt

SEQ ID NO: 53 - DNA, 15 bp, Artificial Sequence
  U1_316 HIV-1 target sequence [NL4-3]
gcggaggctagaagg

SEQ ID NO: 54 - DNA, 17 bp, Artificial Sequence
  U1_316 U1 snRNA target-annealing sequence
atccttctagcctccgc

SEQ ID NO: 55 - DNA, 15 bp, Artificial Sequence
  U1_331 HIV-1 target sequence [NL4-3]
agagagatgggtgcg

SEQ ID NO: 56 - DNA, 17 bp, Artificial Sequence
  U1_331 U1 snRNA target-annealing sequence
atcgcacccatctctct

SEQ ID NO: 57 - DNA, 15 bp, Artificial Sequence
  U1_346 HIV-1 target sequence [NL4-3]
agagcgtcggtatta

SEQ ID NO: 58 - DNA, 17 bp, Artificial Sequence
  U1_346 U1 snRNA target-annealing sequence
attaatactgacgctct

SEQ ID NO: 59 - DNA, 15 bp, Artificial Sequence
  U1_361 HIV-1 target sequence [NL4-3]
agcgggggagaatta

SEQ ID NO: 60 - DNA, 17 bp, Artificial Sequence
  U1_361 U1 snRNA target-annealing sequence
attaattctcccccgct

SEQ ID NO: 61 - DNA, 15 bp, Artificial Sequence
  U1_376 HIV-1 target sequence [NL4-3]
gatcgcgatgggaaa

SEQ ID NO: 62 - DNA, 17 bp, Artificial Sequence
  U1_376 U1 snRNA target-annealing sequence
attttcccatcgcgatc

SEQ ID NO: 63 - DNA, 15 bp, Artificial Sequence
  U1_391 HIV-1 target sequence [NL4-3]
aaattcggttaaggc

SEQ ID NO: 64 - DNA, 17 bp, Artificial Sequence
  U1_391 U1 snRNA target-annealing sequence
atgccttaaccgaattt

SEQ ID NO: 65 - DNA, 15 bp, Artificial Sequence
  U1_690 HIV-1 target sequence [NL4-3]
gatcttcagacctgg

SEQ ID NO: 66 - DNA, 17 bp, Artificial Sequence
  U1_690 U1 snRNA target-annealing sequence
atccaggtctgaagatc

SEQ ID NO: 67 - DNA, 15 bp, Artificial Sequence
  U1_1203 HIV-1 target sequence [NL4-3]
ttacacaagcttaat

SEQ ID NO: 68 - DNA, 17 bp, Artificial Sequence
  U1_1203 U1 snRNA target-annealing sequence
atattaagcttgtgtaa

SEQ ID NO: 69 - DNA, 15 bp, Artificial Sequence
  U1_1546 HIV-1 target sequence [NL4-3]
tagtagacataatag

SEQ ID NO: 70 - DNA, 17 bp, Artificial Sequence
  U1_1546 U1 snRNA target-annealing sequence
atctattatgtctacta

SEQ ID NO: 71 - DNA, 9 bp, Artificial Sequence
  U1_LacZ1 Target sequence
ctacaggaa

SEQ ID NO: 72 - DNA, 11 bp, Artificial Sequence
  U1_LacZ1 U1 snRNA target-annealing sequence
atttcctgtag

SEQ ID NO: 73 - DNA, 9 bp, Artificial Sequence
  U1_LacZ2 Target sequence
tcatctgtg

SEQ ID NO: 74 - DNA, 11 bp, Artificial Sequence
  U1_LacZ2 U1 snRNA target-annealing sequence
atcacagatga

(19) United States
(12) Patent Application Publication
(10) Pub. No.: US 2023/0183742 A1
(43) Pub. Date: Jun. 15, 2023

(54) Viral Vector Production

(71) Applicant: Oxford BioMedica (UK) Limited, Oxford (GB)

(72) Inventors: Rui Andre Saraiva Raposo, Oxford (GB); Charles Moore-Kelly, Oxford (GB); Jordan Wright, Oxford (GB); Daniel Farley, Oxford (GB)

(21) Appl. No.: 17/925,004

(22) PCT Filed: May 14, 2021
(86) PCT No.: PCT/GB2021/051169

§ 371 Date: Nov. 13, 2022

(30) Foreign Application Priority Data
May 15, 2020 (GB) 2007199.9

(51) Int. Cl. C12N 15/86; C12N 5/00; C12N 15/64

(52) U.S. Cl. CPC C12N 15/86; C12N 5/0018; C12N 15/64; C12N 2500/90; C12N 2501/40; C12N 2310/335; C12N 2740/15043; C12N 2740/15051

(57) ABSTRACT

The present invention provides novel methods for producing a viral vector. Corresponding viral vector production systems and uses are also provided.

The present invention provides novel methods for producing a viral vector. Corresponding viral vector production systems and uses are also provided.

### BACKGROUND

The development and manufacture of viral vectors towards vaccines and human gene therapy over the last several decades is well documented in scientific journals and in patents. The use of engineered viruses to deliver transgenes for therapeutic effect is wide-ranging. Contemporary gene therapy vectors based on RNA viruses such as γ-retroviruses and lentiviruses (Muhlebach, M. D. et al., 2010, Retroviruses: Molecular Biology, Genomics and Pathogenesis, 13:347-370; Antoniou, M. N., Skipper, K. A. & Anakok, O., 2013, Hum. Gene Ther., 24:363-374), and DNA viruses such as adenovirus (Capasso, C. et al., 2014, Viruses, 6:832-855) and adeno-associated virus (AAV) (Kotterman, M. A. & Schaffer, D. V., 2014, Nat. Rev. Genet., 15:445-451) have shown promise in a growing number of human disease indications. These include ex vivo modification of patient cells for hematological conditions (Morgan, R. A. & Kakarla, S., 2014, Cancer J., 20:145-150; Touzot, F. et al., 2014, Expert Opin. Biol. Ther., 14:789-798), and in vivo treatment of ophthalmic (Balaggan, K. S. & Ali, R. R., 2012, Gene Ther., 19:145-153), cardiovascular (Katz, M. G. et al., 2013, Hum. Gene Ther., 24:914-927), neurodegenerative diseases (Coune, P. G., Schneider, B. L. & Aebischer, P., 2012, Cold Spring Harb. Perspect. Med., 4:a009431) and tumor therapy (Pazarentzos, E. & Mazarakis, N. D., 2014, Adv. Exp. Med Biol., 818:255-280). As the successes of these approaches in clinical trials begin to build towards regulatory approval and commercialisation, attention has focused on the emerging bottleneck in mass production of good manufacturing practice (GMP) grade vector material (Van der Loo J C M, Wright J F., 2016, Human Molecular Genetics, 25(R1):R42-R52).

A way to overcome this challenge is to find new ways to maximise titre during viral vector production. Common methods of viral vector manufacture include the transfection of primary cells or mammalian/insect cell lines with vector DNA components, followed by a limited incubation period and then harvest of crude vector from culture media and/or cells (Merten, O-W., Schweizer, M., Chahal, P., & Kamen, A. A., 2014, Pharmaceutical Bioprocessing, 2:183-203). In other cases, producer cell lines (PrCLs; where all of the necessary vector component expression cassettes are stably integrated into the production cell DNA) are used during transfection-independent approaches, which is advantageous at larger scales. The efficiency of viral vector manufacturing is typically affected by several factors at the ‘upstream phase’, including [1] viral serotype/pseudotype employed, [2] transgenic sequence composition and size, [3] media composition/gassing/pH, [4] transfection reagent/process, [5] chemical induction and vector harvest timings, [6] cell fragility/viability, [7] bioreactor shear-forces and [8] impurities. Clearly there are also other factors to consider during the ‘downstream’ purification/concentration phase (Merten, O-W. et al., 2014, Pharmaceutical Bioprocessing, 2:237-251).

Thus, there is a need in the art to provide alternative methods of producing viral vectors which help to address the known issues associated with the mass production of GMP grade vector material.

### BRIEF SUMMARY OF THE DISCLOSURE

The inventors have surprisingly shown that use of a PKC activator alone or in combination with a HDAC inhibitor during viral vector production significantly increases viral vector titre. The invention therefore relates to the use of a PKC activator i) on its own as an inducer of viral vector production, and ii) as an enhancer of HDAC inhibitor induction of viral vector production.

The inventors have also shown that cells treated with a PKC activator maintain high cell viabilities, which is beneficial during viral vector production.

Accordingly, a method for producing a viral vector is provided, the method comprising culturing a cell comprising nucleic acid sequences encoding viral vector components in a cell culture medium that comprises a PKC activator.

Suitably, the viral vector may be a self-inactivating viral vector.

Suitably, the PKC activator may be prostratin or phorbol 12-myristate 13-acetate, an analogue, derivative or pharmaceutically acceptable salt thereof.

Suitably,

a) prostratin may be in the cell culture medium at a concentration of at least about 0.5 μM, optionally wherein prostratin may be at a concentration of from about 0.5 to about 32 μM; or

b) phorbol 12-myristate 13-acetate may be in the cell culture medium at a concentration of at least about 1 nM, optionally wherein phorbol 12-myristate 13-acetate may be at a concentration of from about 1 to about 32 nM.

Suitably, the viral vector may be a lentiviral vector and a modified U1 snRNA may be co-expressed with the lentiviral vector components, wherein said modified U1 snRNA binds to a nucleotide sequence within the packaging region of the lentiviral vector genome sequence.

Suitably, the viral vector may be a lentiviral vector and splicing activity from the major splice donor region of the lentiviral vector may have been functionally ablated.

Suitably, the viral vector may be a lentiviral vector, wherein the lentiviral vector genome has been mutated in the major splice donor region or mutated in the major splice donor region and at least one cryptic splice donor region.

Suitably, the cell culture medium may further comprise a HDAC inhibitor.

Suitably, the HDAC inhibitor may be an aliphatic HDAC inhibitor or a hydroxamic acid HDAC inhibitor.

Suitably, the aliphatic HDAC inhibitor may be sodium butyrate, sodium valproate or valeric acid, an analogue, derivative or pharmaceutically acceptable salt thereof.

Suitably, the PKC activator may be prostratin and the HDAC inhibitor may be sodium butyrate.

Suitably, the hydroxamic acid HDAC inhibitor may be suberanilohydroxamic acid, an analogue, derivative or pharmaceutically acceptable salt thereof.

Suitably,

a) sodium butyrate may be in the cell culture medium at a concentration of at least about 2.5 mM, optionally wherein sodium butyrate may be at a concentration of from about 2.5 to about 30 mM;

b) sodium valproate may be in the cell culture medium at a concentration of at least about 3 mM, optionally wherein sodium valproate may be at a concentration of from about 3 to about 30 mM;

c) valeric acid may be in the cell culture medium at a concentration of at least about 3 mM, optionally wherein valeric acid may be at a concentration of from about 3 to about 30 mM; or

d) suberanilohydroxamic acid may be in the cell culture medium at a concentration of at least about 0.5 μM, optionally wherein suberanilohydroxamic acid may be at a concentration of from about 0.5 to about 16 μM.

Suitably, the cell may be a transiently transfected production cell. In this context, the nucleic acid sequences encoding the viral vector components are transiently transfected into the production cell.

Suitably, the cell may be a stable producer cell. In this context, the nucleic acid sequences encoding the viral vector components are stably integrated into the producer cell.

Suitably, the cell may be a eukaryotic cell.

Suitably, the cell may be a mammalian cell.

Suitably, the cell may be a human cell.

Suitably, the cell may be adherent.

Suitably, the cell may be a HEK293 cell, or a derivative thereof.

Suitably, the HEK293 production cell may be a HEK293T cell.

Suitably, the cell may be in suspension.

Suitably, the viral vector may be selected from the group consisting of: a retroviral vector, an adenoviral vector, an adeno-associated viral vector, a herpes simplex viral vector and a vaccinia viral vector.

Suitably, the retroviral vector may be a lentiviral vector.

Suitably, the lentiviral vector may be selected from the group consisting of: HIV-1, HIV-2, SIV, FIV, BIV, EIAV, CAEV and visna lentiviral vector.

Suitably, the viral vector may comprise a nucleotide of interest (NOI).

Suitably, the cell culture medium may comprise a volume of at least about 5 litres of medium.

Suitably, the cell culture medium may be serum-free.

Suitably, at least one nucleic acid sequence encoding a viral vector component may be operably linked to a promoter selected from the group consisting of: a CMV promoter, an RSV promoter, a CAG synthetic promoter, a CHEF1 promoter, a GRP78 promoter, a UBC promoter, an HIV-1 U3 promoter, and a FERH promoter, optionally wherein the promoter may be selected from the group consisting of: a CMV promoter, an RSV promoter, and a CAG synthetic promoter.

A viral vector production system is also provided, comprising:

i) a cell comprising nucleic acid sequences encoding viral vector components; and

ii) a cell culture medium that comprises a PKC activator.

Suitably, the viral vector may be a self-inactivating viral vector.

Suitably, the PKC activator may be prostratin or phorbol 12-myristate 13-acetate, an analogue, derivative or pharmaceutically acceptable salt thereof.

Suitably:

a) prostratin may be in the cell culture medium at a concentration of at least about 0.5 μM, optionally wherein prostratin may be at a concentration of from about 0.5 to about 32 μM; or

b) phorbol 12-myristate 13-acetate may be in the cell culture medium at a concentration of at least about 1 nM, optionally wherein phorbol 12-myristate 13-acetate may be at a concentration of from about 1 to about 32 nM.

Suitably, the viral vector production system may further comprise a nucleic acid sequence encoding a modified U1 snRNA, wherein the modified U1 snRNA binds to a nucleotide sequence within the packaging region of the lentiviral vector genome sequence.

Suitably, the viral vector may be a lentiviral vector, wherein splicing activity from the major splice donor region of the lentiviral vector genome has been functionally ablated.

Suitably, the viral vector may be a lentiviral vector and wherein the lentiviral vector genome has been mutated in the major splice donor region or mutated in the major splice donor region and at least one cryptic splice donor region.

Suitably, the cell culture medium may further comprise a HDAC inhibitor.

Suitably, the HDAC inhibitor may be an aliphatic HDAC inhibitor or a hydroxamic acid HDAC inhibitor.

Suitably, the aliphatic HDAC inhibitor may be sodium butyrate, sodium valproate or valeric acid, an analogue, derivative or pharmaceutically acceptable salt thereof.

Suitably, the PKC activator may be prostratin and the HDAC inhibitor may be sodium butyrate.

Suitably, the hydroxamic acid HDAC inhibitor may be suberanilohydroxamic acid, an analogue, derivative or pharmaceutically acceptable salt thereof.

Suitably:

a) sodium butyrate may be in the cell culture medium at a concentration of at least about 2.5 mM, optionally wherein sodium butyrate may be at a concentration of from about 2.5 to about 30 mM;

b) sodium valproate may be in the cell culture medium at a concentration of at least about 3 mM, optionally wherein sodium valproate may be at a concentration of from about 3 to about 30 mM;

c) valeric acid may be in the cell culture medium at a concentration of at least about 3 mM, optionally wherein valeric acid may be at a concentration of from about 3 to about 30 mM; or

d) suberanilohydroxamic acid may be in the cell culture medium at a concentration of at least about 0.5 μM, optionally wherein suberanilohydroxamic acid may be at a concentration of from about 0.5 to about 16 μM.

Suitably, the cell may be a transiently transfected production cell.

Suitably, the cell may be a stable producer cell.

Suitably, the cell may be a eukaryotic cell.

Suitably, the cell may be a mammalian cell.

Suitably, the cell may be a human cell.

Suitably, the cell may be adherent.

Suitably, the cell may be a HEK293 cell, or a derivative thereof.

Suitably, the HEK293 production may be a HEK293T cell.

Suitably, the cell may be in suspension.

Suitably, the viral vector may be selected from the group consisting of: a retroviral vector, an adenoviral vector, an adeno-associated viral vector, a herpes simplex viral vector and a vaccinia viral vector.

Suitably, the retroviral vector may be a lentiviral vector.

Suitably, the lentiviral vector may be selected from the group consisting of: HIV-1, HIV-2, SIV, FIV, BIV, EIAV, CAEV and visna lentiviral vector.

Suitably, the viral vector may comprise a nucleotide of interest (NOI).

Suitably, the cell culture medium may be serum-free.

Suitably, at least one nucleic acid sequence encoding a viral vector component may be operably linked to a promoter selected from the group consisting of: a CMV promoter, an RSV promoter, a CAG synthetic promoter, a CHEF1 promoter, a GRP78 promoter, a UBC promoter, an HIV-1 U3 promoter, and a FERH promoter, optionally wherein the promoter may be selected from the group consisting of: a CMV promoter, an RSV promoter, and a CAG synthetic promoter.

In the context of the method or viral vector production system described herein, nucleic acid sequences encoding viral vector components may encode the viral vector components required for the production of a lentiviral vector. For example, they may encode i) gag-pol; ii) env; iii) the viral vector genome (typically encoding the NOI) and iv) optionally rev, or a functional substitute thereof, wherein the env may be VSV-G env. Each nucleic acid sequence of i) to iv) may be a separate or may be part of a module construct. For example, at least two of the nucleic acid sequences of i) to iv) may be modular constructs encoding the viral vector components located at the same genetic locus. In a further example, at least two of the nucleic acid sequences may be modular constructs encoding the viral vector components in reverse and/or alternating orientations. In the yet further example, at least two of the nucleic acid sequences are modular constructs encoding gag-pol and/or env, wherein the modular constructs are associated with at least one regulator element.

The nucleic acid sequences encoding viral vector components may alternatively encode the viral vector components required for the production of a different retroviral vector, or the viral components required for the production of an adeno-associated viral vector, a herpes simplex viral vector or a vaccinia viral vector. The functional components required for the production of each of these viral vectors is well known in the art. For example, for the production of AAV vectors, a nucleic acid sequence encoding a capsid protein may be used, The use of a PKC activator for increasing viral vector titre during viral vector production is also provided.

Suitably, the PKC activator may be used in combination with a HDAC inhibitor.

Suitably, the viral vector may be a self-inactivating viral vector.

Suitably, the PKC activator may be prostratin or phorbol 12-myristate 13-acetate, an analogue, derivative or pharmaceutically acceptable salt thereof.

Suitably, the HDAC inhibitor may be an aliphatic HDAC inhibitor or a hydroxamic acid HDAC inhibitor.

Suitably, the aliphatic HDAC inhibitor may be sodium butyrate, sodium valproate or valeric acid, an analogue, derivative or pharmaceutically acceptable salt thereof.

Suitably, the PKC activator may be prostratin and the HDAC inhibitor may be sodium butyrate.

Suitably, the hydroxamic acid HDAC inhibitor may be suberanilohydroxamic acid, an analogue, derivative or pharmaceutically acceptable salt thereof.

Suitably, the viral vector may be produced from a cell comprising nucleic acid sequences encoding viral vector components, wherein at least one of the nucleic acid sequences is operably linked to a promoter selected from the group consisting of: a CMV promoter, an RSV promoter, a CAG synthetic promoter, a CHEF1 promoter, a GRP78 promoter, a UBC promoter, an HIV-1 U3 promoter, and a FERH promoter, optionally wherein the promoter may be selected from the group consisting of: a CMV promoter, an RSV promoter, and a CAG synthetic promoter.

Various aspects of the invention are described in further detail below.

### BRIEF DESCRIPTION OF THE FIGURES

Embodiments of the invention are further described hereinafter with reference to the accompanying drawings, in which:

FIG. 1 shows (A) a schematic of the typical configuration of a third generation (Self-inactivating (SIN)) lentiviral vector expression cassette, containing a functional major splice donor embedded within stem loop (SL2) of the packaging signal, and the types of mRNA generated during lentiviral vector production. The types of mRNA generated from a ‘standard’ lentiviral vector (LV) DNA cassette and a lentiviral vector DNA cassette with (a) functional mutation(s) in the MSD region (‘MSD-KO LV DNA cassette’) that suppress or ablate the promiscuous activity from the MSD are shown. For both cassettes, the full-length (Unspliced) vector RNA (vRNA) results from the co-expression of rev, which binds to the rev response element (RRE), and is generally believed to repress splicing from the MSD to splice acceptor 7 (sa7) included with the RRE sequence. For a standard lentiviral vector DNA cassette, in the absence of rev, it is generally believed that splicing-out of all introns occurs efficiently (Spliced). However, ‘aberrant’ splice products can be made during lentiviral vector production wherein the MSD highly efficiently splices to splice acceptor sites or cryptic splice acceptor sites (“Aberrant′ spliced”), typically ‘over-looking’ the RRE-containing intron such that rev has minimal impact on this activity of the MSD. Lentiviral vector production can also be performed with co-expression of modified U1 snRNAs redirected to the packaging region of MSD-mutated lentiviral vector DNA cassettes. (Key: Pro, promoter; region from 5′R to gag contains the packaging element {Ψ}; msd, major splice donor; cppt, central polypurine tract; Int, intron; sd/sa, splice donor/acceptor; GOI, gene of interest; grey arrow indicate position of forward {f} and reverse {r} primers to assess the proportion of Unspliced vRNA produced during 3^{rd} generation lentiviral vector production. Post-transcriptional regulatory element {PRE} not shown for clarity). (B) U3 is removed from LTR of SIN LV vectors (C) 3^{rd} gen CMV driven LV vector plasmids.

FIG. 2 shows final vector titres from HEK293T cells induced with varying concentrations of antioxidant (NAC), HDAC inhibitors: sodium butyrate, sodium valproate, valeric acid, SAHA and TSA; HAT inhibitor (tannic acid); transcriptional activators: PMA, HMBA and prostratin.

FIG. 3 shows final vector titres from HEK293T cells induced with randomised combinations of NAC; HDAC inhibitors: sodium butyrate, sodium valproate, valeric acid, SAHA, TSA; HAT inhibitor (tannic acid) and transcriptional activators: PMA, HMBA and prostratin. Dotted line indicates level of induction at 20 mM sodium butyrate.

FIG. 4 shows final vector titres from HEK293T cells induced with varying concentrations of HDAC inhibitors: sodium butyrate, sodium valproate, valeric acid and SAHA; transcriptional activators: HMBA, prostratin and PMA; and HDAC inhibitors in combination with transcriptional activators. Arrows indicate the induction agent concentrations used in combination.

FIG. 5 shows (A) final vector titre from HEK293T cells induced with varying concentrations of sodium butyrate, prostratin and HMBA. (B) JMP prediction profiler of vector titre results.

FIG. 6 shows JMP prediction profiler of vector titre results for (A) sodium valproate, (B) valeric acid and (C) SAHA.

FIG. 7 shows final vector titre from HEK1.65s cells induced with varying concentrations of sodium butyrate, sodium valproate, valeric acid and SAHA with and without prostratin.

FIG. 8 shows final vector titre from HEK1.65s cells induced with varying concentrations of prostratin and sodium butyrate.

FIG. 9 shows (A) Surface plot of interaction between sodium butyrate and prostratin on vector titre. (B) DOE Actual by Predicted plot, Effect Summary and Lack of Fit table, and (C) Prediction Profiler.

FIG. 10 shows viral titre determined by (A) FACS and (B) duplex integration QPCR assays.

FIG. 11 is a schematic of a U1 snRNA molecule and an example of how to modify the targeting sequence for use in the invention. The endogenous non-coding RNA, U1 snRNA binds to the consensus splice donor site (5′-MAGGURR-3′ (SEQ ID NO: 1)) via the 5′-(AC)UUACCUG-3′ (SEQ ID NO:2) (grey highlighted) native splice donor targeting sequence during early steps of intron splicing. Stem loop I binds to U1A-70K protein that has been shown to be important for polyA suppression. Stem loop II binds to U1A protein, and the 5′-AUUUGUGG-3′ (SEQ ID NO: 3) sequence binds to Sm proteins, which together with Stem loop IV, is important for U1 snRNA processing. In the invention, the modified U1 snRNA is modified to introduce a heterologous sequence that is complementary to a target sequence within the vector genome vRNA molecule at the site of the native splice donor targeting sequence; in this figure the example given directs the modified U1 snRNA to 15 nucleotides (256-270 relative to the first nucleotide of the vector genome molecule, 256U1) of a standard HIV-1 lentiviral vector genome (located in the SL1 loop if the packaging signal).

FIG. 12: Implications of aberrant splicing from the major splice donor site (MSD) within HIV-1 based lentiviral vectors. (A) A schematic to show the typical configuration of a third generation (Self-inactivating (SIN)) lentiviral vector expression cassette is shown in FIG. 1. In FIG. 12, standard 3rd generation lentiviral vector production was performed +/−rev in HEK293T cells and total RNA extracted from post-production cells. Total RNA was subjected to qPCR (SYBR green) using two primer sets (position marked in A): f+rT amplified total transcripts generated from the lentiviral vector expression cassette, and f+rUS amplified Unspliced transcripts; therefore the proportion of Unspliced-to-Total vRNA transcripts were calculated and plotted. The data indicates that the proportion of Unspliced vRNA relative to total during standard 3rd generation lentiviral vector production is modest and varies according to the internal transgene cassette (in this case containing different promoters and the GFP gene); moreover, this proportion is only minimally increased by the action of rev.

FIG. 13: (A) A schematic to show the configuration of standard or MSD-mutated lentiviral vector expression cassettes encoding an EF1a-GFP internal expression cassette, and the types of mRNA generated during lentiviral vector production. (Key: Pro, promoter; region from 5′R to gag contains the packaging element {Ψ}; msd, major splice donor; cppt, central polypurine tract; Int, intron; sd/sa, splice donor/acceptor; GOI, gene of interest; grey arrow indicate position of forward {f} and reverse {r} primers to assess the proportion of Unspliced vRNA produced during 3rd generation lentiviral vector production. Post-transcriptional regulatory element {PRE} not shown for clarity). (B) [i] The standard lentiviral vectors or MSD-2KO lentiviral vectors were produced in HEK293T cells +/−tat, or 179U1, or 305U1, and titrated. [ii] Total cytoplasmic mRNA was extracted from post-production cells and analysed by RT-PCR/gel electrophoreses using primers (f+rG) that could detect the main ‘aberrant’ splice product from the SL2 splicing region SL2 splicing region to the EF1a splice acceptor. The data show that modified U1 snRNAs redirected to the 5′ packaging region of MSD-2KO lentiviral vector genome (vRNA) were able to increase titres of both standard and MSD-2KO lentiviral vectors in a manner similar to tat. The MSD-2KO mutation abolished detection of the ‘aberrant’ splice product, which is from the SL2 splicing region to the EF1a splice acceptor (see FIG. 14A). Importantly, the increase in titres by the modified U1 snRNAs was accompanied by maintenance of virtually undetectable ‘aberrant’ spliced product, in contrast to the use of tat.

FIG. 14: A description of functional major splice donor mutations, their impact on lentiviral vector titres, and recovery by modified U1 snRNA. (A) The sequence of the stem loop 2 (SL2) region of ‘wild type’ HIV-1 (NL4-3; the ‘standard’ sequence within current lentiviral vector genomes) is shown at the top. The sequence comprises the major splice donor site (MSD: consensus=CTGGT) and a cryptic splice donor site (that is utilized when the MSD site is mutated on its own (crSD: consensus=TGAGT)). The nucleotides at the position of splicing when the splice donor site is used are identified in bold and by arrows. Four functional MSD mutations that ablate both the MSD and the crSD site splicing activities are described: MSD-2KO, which mutates the two ‘GT’ motifs from the MSD and the crSD; MSD-2KOv2, which also comprises mutations that ablate both the MSD and crSD sites; MSD-2KOm5, which introduces an entirely new stem-loop structure lacking any splice donor sites; and ΔSL2, which deletes the SL2 sequence entirely. The substitutions introduced to the SL2 sequence in the MSD-2KO, MSD-2KOv2 and MSD-2KOm5 mutations are shown in lowercase italics. (B) The four lentiviral vector genome variants comprising functional MSD mutations (described in FIG. 14A) were cloned with EFS-GFP internal cassettes, and MSD-2KO or MSD-2KOm5 variants additionally cloned with EF1a-, CMV- or huPGK-GFP internal cassettes. Standard and MSD-mutated LVs were produced in HEK293T cells +/−256U1, and titrated. The data indicates that the degree of attenuation of lentiviral vector titre can vary according to the specific mutation, and that the MSD-2KOm5 variant generally produced a less attenuated phenotype. The modified U1 snRNA was capable of increasing lentiviral vector titres for the four lentiviral vector genome variants comprising functional MSD mutations when co-expressed during production. Titre increases were greatest when the 256U1 was expressed with MSD-mutated LV genomes harbouring the MSD-2KOm5 sequence.

FIG. 15: The use of Prostratin alone or in combination with modified U1 snRNA to enhance production titres of lentiviral vectors (LVs) harbouring functional mutations within the major splice donor (MSD) region. The effect of mutating the MSD, and the cryptic splice site immediately downstream of the MSD, is the reduction in production titres, due to a reduction in vector RNA (vRNA) production. The titres of MSD-mutated LVs can be restored by supplying a modified U1 snRNA such that it can anneal to a region within the packaging region of the vRNA, thus increasing the pool of packageable vRNA. To test whether the supply of Prostratin during MSD-mutated LV production might also boost titres, HIV-MSD2KOm5-EFS-GFP (A) or HIV-MSD2KOm5-EF1a-GFP (B) was produced in serum-free suspension HEK293T cells in the absence of inducers, or with 11 μM Prostratin (added at the sodium butyrate step) or with co-transfection with a plasmid expressing the ‘256U1’ modified U1 snRNA. Surprisingly, Prostratin increased titres of the MSD-mutated LV vectors by 5-10 fold, and when Prostratin and 256U1 were applied together the titres achieved were higher that standard LV production titres produced in the absence of inducers.

FIG. 16 shows vector titres of CAR #1, CAR #2 and CAR #2-T2A-GFP produced in transiently transfected HEK1.65s cells in absence of titre enhancing agents, with 256U1 expression, with 11 μM prostratin at induction, and with 256U1 expression combined with 11 μM prostratin at induction.

FIG. 17 shows vector titres (TU/mL) at harvest for the production of EIAV-CMV-GFP with and without 11 μM prostratin at induction.

FIG. 18 shows induction of promoters of different strengths by prostratin. Suspension (serum-free) HEK293T cells were transfected with plasmids encoding a GFP reporter gene driven by the stated promoters. To model expression of a viral vector component (e.g. AAV capsid, LV genome) during production, two different plasmid input amounts were performed (0.1 μg/mL [Lo] and 0.95 μg/mL [Hi]), and all cultures were induced with 10 mM sodium butyrate with or without 11 μM prostratin post-transfection. Approximately 2 days post-transfection, cells were analysed by flow cytometry to measure GFP expression. Transgene expression scores (& GFP-positive×median fluorescence intensity) were generated for each condition, and plotted on a linear y-axis (note the two sets of graphs' y-axis ranges differ in magnitude by log-10 from top to bottom i.e. strongest promoters to weakest). Cytomegalovirus promoter—CMV, Rous Sarcoma virus U3 promoter—RSV, CAG synthetic promoter (CMV enhancer, promoter-exon/intron of chicken beta-actin gene, the splice acceptor of the rabbit beta-globin gene), Chinese hamster EF-1alpha-1 promoter—CHEF1, GRP78/BiP (stress-inducible) promoter—GRP78, Ubiquitin-C promoter—UBC, HIV-1 U3 promoter—HIV-1 U3, Human ferritin heavy chain promoter—FERH, Untransfected control—UTC.

Various aspects of the invention are described in further detail below.

### DETAILED DESCRIPTION

The inventors have identified that PKC activators may be used to increase viral vector titres during viral vector production. In addition, they have shown that cell viability is maintained when a PKC activator is present during viral vector production. The methods, viral vector production systems, and uses described herein therefore comprise the use of PKC activators as described in more detail below.

A. PKC Activator

(i) Methods for Producing a Viral Vector

A method for producing a viral vector is provided, the method comprising culturing a cell comprising nucleic acid sequences encoding viral vector components in a cell culture medium that comprises a PKC activator.

The terms “cell”, “culture”, “cell culture”, “cell culture medium”, “nucleic acid sequence”, “viral vector” and “viral vector components” are described in more detail elsewhere herein and apply equally here.

The cells used in the methods described herein may be transiently transfected production cells or stable producer cells. The terms “transiently transfected production cell” and “stable producer cell” are described in more detail elsewhere herein and apply equally here.

The cell may be a eukaryotic cell, such as a mammalian cell (e.g. a human cell). Alternative cell types are discussed in more detail below.

The cells may be adherent or in suspension. Suitable cell types are discussed in more detail elsewhere herein, and include HEK293 cells (e.g. HEK293T cells), or derivatives thereof.

The methods described herein may be used for the production of any suitable viral vector. Appropriate viral vectors are described in more detail in the definitions section herein and apply equally here. Examples of viral vectors that may be produced by the methods are described herein include a viral vector selected from the group consisting of: a retroviral vector, an adenoviral vector, an adeno-associated viral vector, a herpes simplex viral vector and a vaccinia viral vector. Details of each of these vectors is provided elsewhere and applies equally here.

The methods described herein are particularly suitable for the production of a retroviral vector, particularly a lentiviral vector. For example, the methods described herein may be used for the production of a lentiviral vector selected from the group consisting of: HIV-1, HIV-2, SIV, FIV, BIV, EIAV, CAEV and visna lentiviral vector. In one example, the methods described herein may be used for the production of a lentiviral vector selected from an HIV (e.g. HIV-1, HIV-2) or an EIAV lentiviral vector.

Each of these lentiviral vectors is described in more detail elsewhere herein.

The methods provided herein are particularly useful when producing self-inactivating (SIN) viral vectors (for example, SIN lentiviral vectors). The characteristics of SIN vectors are described in more detail elsewhere herein. In a particular example, the SIN vector may be a 3^{rd} generation SIN viral vector (e.g. a 3^{rd} generation lentiviral vector).

Typically, the viral vectors produced by the methods described herein comprise a nucleotide of interest (NOI). The NOI may be any suitable NOI. Examples of appropriate NOIs are provided elsewhere herein.

Typically, in some examples, the nucleic acid sequences encoding viral vector components encode vector components including gag-pol, env, optionally rev, and the genome of the viral vector. Further details are provided elsewhere herein.

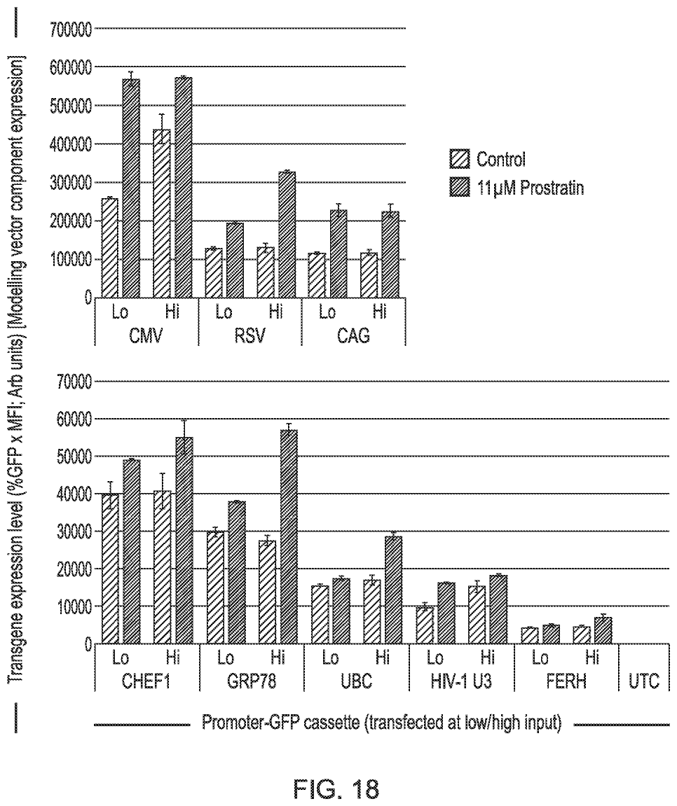

The inventors have shown that the addition of a PKC activator (such as prostratin) increases viral vector titre (and maintains cell viability) irrespective of which promoter is used (i.e. the effect is not promoter specific). The inventors have tested several different promoters to demonstrate that the effects observed herein are independent of the promoter that is used. By way of example, the inventors have shown that the methods of the invention are compatible with the use of CMV (Cytomegalovirus), CHEF-1 (CHO-derived elongation factor 1), RSV (Rous Sarcoma Virus) and GRP78 (Immunoglobulin heavy chain-binding protein) promoters (for driving GFP genome, Gag/Pol, Rev and VSVG plasmid expression respectively). In addition, the inventors have demonstrated that the following promoters can be used when inducing GFP plasmid expression with prostratin: Cytomegalovirus promoter—CMV, Rous Sarcoma virus U3 promoter—RSV, CAG synthetic promoter (CMV enhancer, promoter-exon/intron of chicken beta-actin gene, the splice acceptor of the rabbit beta-globin gene), Chinese hamster EF-1alpha-1 promoter—CHEF1, GRP78/BiP (stress-inducible) promoter—GRP78, Ubiquitin-C promoter—UBC, HIV-1 U3 promoter—HIV-1 U3, and Human ferritin heavy chain promoter—FERH (see FIG. 18). These promoters can be used to drive viral vector production of several different types of viral vector including a viral vector selected from the group consisting of: a retroviral vector, an adenoviral vector, an adeno-associated viral vector, a herpes simplex viral vector and a vaccinia viral vector. Strong promoters such as CMV, RSV and CAG, for example, may be selected for use in driving expression of viral vector components, such as structural viral vector components, including the AAV capsid protein. The PKC activators described herein can advantageously be used to increase viral vector production when one or more of these promoters is used. The invention therefore provides for the use of a PKC activator for increasing viral vector titre during viral vector production from a cell comprising nucleic acid sequences encoding viral vector components, wherein at least one of the nucleic acid sequences is operably linked to a promoter selected from the group consisting of: a CMV promoter, an RSV promoter, a CAG synthetic promoter, a CHEF1 promoter, a GRP78 promoter, a UBC promoter, an HIV-1 U3 promoter, and a FERH promoter. Optionally, the promoter may be selected from the group consisting of: a CMV promoter, an RSV promoter, and a CAG synthetic promoter. This is particularly relevant for nucleic acid sequences encoding structural viral vector components (such as the AAV capsid protein), when the use of strong promoters is desirable. Optionally the viral vector may be a retroviral vector (e.g. a lentiviral vector), an adenoviral vector, or an adeno-associated viral vector.

The invention therefore provides for the use of a PKC activator for increasing viral vector titre during viral vector production from a cell comprising nucleic acid sequences encoding viral vector components, wherein at least one of the nucleic acid sequences is operably linked to a promoter selected from the group consisting of: a CMV promoter, an RSV promoter, a CAG synthetic promoter, a CHEF1 promoter, a GRP78 promoter, a UBC promoter, an HIV-1 U3 promoter, and a FERH promoter, and wherein the viral vector is selected from the group consisting of: a retroviral vector, an adenoviral vector, an adeno-associated viral vector, a herpes simplex viral vector and a vaccinia viral vector. Optionally, the promoter may be selected from the group consisting of: a CMV promoter, an RSV promoter, and a CAG synthetic promoter. Optionally the viral vector may be a retroviral vector (e.g. lentiviral vector), an adenoviral vector, or an adeno-associated viral vector.

The inventors have identified that the presence of a PKC activator in the cell culture medium during viral vector production increases viral vector titre. The cell culture medium used in the methods described herein may therefore be any suitable cell culture medium, provided that it comprises a PKC activator. Suitable cell culture media and cell culture methodology is described in more detail elsewhere herein and applies equally here.

In one particular example, the cell culture medium may be serum free. As used herein “serum free conditions” are conditions in which serum is omitted from the culture medium such that e.g. the culture medium does not comprise (i.e. is essentially free from or not supplemented with) serum.

As used herein, “protein kinase C activator” or “PKC activator” refers to a substance that increases the rate of the reaction catalyzed by PKC. Several PKC activators and methods of identifying PKC activators are well known in the art. Examples of appropriates methods of identifying PKC activators are provided in Chakravarthy et al., Analytical Biochemistry, Vol 196, Issue 1, 1991, pp 144-150). A PKC activator is also referred to herein as a PKC agonist.

Protein kinase C (PKC) is one of the largest gene families of protein kinases. The protein kinase C (PKC) family of serine-threonine kinases plays an important regulatory role in a variety of biological phenomena. The PKC family is composed of at least 12 individual isoforms which belong to 3 distinct categories: (i) conventional isoforms 25 (α, β1, β2, γ) activated by Ca2+, phorbol esters and diacylglycerol liberated intracellularly by phospholipase C; (ii) novel isoforms (δ, η, ε, θ) which are also activated by phorbol esters and diacylglycerol but not by Ca2+; and (iii) atypical (ζ, λ, ι) members of the family, which are not activated by Ca2+, phorbol esters or diacylglycerol. The identity of protein kinase C is generally established by its ability to phosphorylate proteins when adenosine triphosphate and phospholipid cofactors are present, with greatly reduced activity when these cofactors are absent. Additionally, some forms of protein kinase C require the presence of calcium ions for maximal activity. Protein kinase C activity is also substantially stimulated by certain 1,2-sn-diacylglycerols that bind specifically and stoichiometrically to a recognition site on the enzyme. On activation, most but not all isoforms are thought to translocate to the plasma membrane from the cytoplasm. Numerous studies have characterized the structure and function of PKC because of its importance in a wide variety of biological processes.

PKC activators can be non-specific or specific activators. A specific activator activates one PKC isoform, e.g., PKC-c (epsilon), to a greater detectable extent than another PKC isoform. Exemplary PKC activators are disclosed in WO 2017/062924 A1, in particular at paragraphs [039], [040], [053], [058]-[0112], the entire content of which is incorporated by reference herein. Wu-Zhang and Newton, Biochem. J. (2013) 452, 195-209, the entire content of which is incorporated by reference herein, also discloses exemplary PKC activators.

A PKC activator may be selected from prostratin, phorbol 12-myristate 13-acetate, macrocyclic lactones, bryologs, diacylglcerols, isoprenoids, octylindolactam, gnidimacrin, ingenol, iripallidal, napthalenesulfonamides, diacylglycerol inhibitors, growth factors, polyunsaturated fatty acids, monounsaturated fatty acids, cyclopropanated polyunsaturated fatty acids, cyclopropanated monounsaturated fatty acids, fatty acids alcohols and derivatives, and fatty acid esters, or a pharmaceutically acceptable salt or derivative thereof.

The PKC activator may be prostratin or phorbol 12-myristate 13-acetate (PMA), or an analogue, derivative or pharmaceutically acceptable salt thereof. The PKC activator may be a macrocyclic lactone, e.g. comprising a 14-, 15-, or 16-membered lactone ring. The macrocyclic lactone may be a bryostatin (such as Bryostatin-1, Bryostatin-2, Bryostatin-3, Bryostatin-4, Bryostatin-5, Bryostatin-6, Bryostatin-7, Bryostatin-8, Bryostatin-9, Bryostatin-10, Bryostatin-11, Bryostatin-12 Bryostatin-13, Bryostatin-14, Bryostatin-15, Bryostatin-16, Bryostatin-17, and/or Bryostatin-18); a neristatin (such as neristatin-1); a macrocylic derivative of cyclopropanated polyunsaturated fatty acids (such as 24-octaheptacyclononacosan-25-one); a bryolog (analogue of a bryostatin). The PKC activator may be a diacylglcerol (or derivative thereof) that binds to and activates PKC. The PKC activator may be an isoprenoid, such as farnesyl thiotriazole. The PKC activator may be octylindolactam V, gnidimacrin, ingenol, or iripallidal. The PKC may be a napthalenesulfonamide, such as N-(n-heptyl)-5-chloro-1-naphthalenesulfonamide, or N-(6-phenylhexyl)-5-chloro-1-naphthalenesulfonamide. The PKC activator may be a diacylglycerol kinase inhibitor, which indirectly activates PKC, (such as 6-(2-(4-[R4-fluorophenyl)phenylmethylene]-1-piperidinyl)ethyl)-7-methyl-5H-thiazolo[3,2-a]pyrimidin-5-one (R59022), or [3-[2-[4-(bis-(4-fluorophenyl)methylene]piperidin-1-yl)ethyl]-2,3-dihydro-2-thioxo-4(1)-quinazolinone (R59949)). The PKC activator may be a growth factor (such as fibroblast growth factor 18 (FGF-18), insulin growth factor, 4-methylcatechol acetic acid, NGF, or BDNF). The PKC activator may be a polyunsaturated fatty acid, a monounsaturated fatty acid, a cyclopropanated polyunsaturated fatty acid, a cyclopropanated monounsaturated fatty acid, a fatty acid alcohol, a cyclopropanated polyunsaturated fatty acid alcohol, a cyclopropanated monounsaturated fatty acid alcohol, a fatty acid ester, a cyclopropanated polyunsaturated fatty acid ester, or a cyclopropanated monounsaturated fatty acid ester.

Prostratin is also known as 12-Deoxyphorbol-13-acetate (≥98% HPLC, Sigma: P0077). Prostratin was initially isolated at the National Cancer Institute (NCI) as the active constituent of extracts of the tropical plant, Homalanthus nutans, which was used in traditional Samoan herbal medicine for treatment of “yellow fever,” i.e., hepatitis (Gustafson et al., 1992, J Med Chem 35(11): 1978-86). In contrast to other phorbol esters, prostratin is a potent anti-tumor agent. Prostratin and structural analogues thereof may be purified from a natural source or may be synthetically made. Methods for synthetically producing prostratin and structural analogues are known in the art (Wender et al., 2008, Science 320(5876): 649-52).

Phorbol 12-myristate 13-acetate (PMA) is also known as 12-O-Tetradecanoylphorbol-13-acetate (TPA). Phorbol 12-myristate 13-acetate is a potent tumor promoter and activates protein kinase C in vivo and in vitro. It is a phorbol ester that is associated with many cellular responses including gene transcription, cell division and differentiation, apoptosis and immune response.

The PKC activator may be present in the cell culture medium at any suitable concentration. A range of suitable concentrations may readily be identified by a person of skill in the art, using routine experimentation. For example, methods similar to those in the examples section below may be used to identify a PKC activator concentration that increases viral titre. Several methods for measuring viral titre are known in the art. Further details are provided elsewhere herein.

The cells may be cultured in the presence of the PKC activator in any appropriate cell culture vessel, using any appropriate cell culture volume. Cell culture tubes, cell culture flasks, cell culture dishes and cell culture plates are referred to herein as cell culture vessels as they are examples of discrete cell culture products (or consumables) that may be used within the methods described herein. Cell culture tubes, cell culture flasks, cell culture dishes are typically cell culture vessels with a single cell culture reaction chamber, whereas cell culture plates are typically cell culture vessels with several cell culture reaction chambers (i.e. several wells). Other appropriate cell culture vessels are well known in the art.

The cells may be cultured in the presence of the PKC activator for an appropriate duration of time. Typically, cells are cultured in the presence of the PKC activator for a duration of at least 30 minutes. In other words, the cells may be cultured in the presence of the PKC activator for a duration of at least 30 minutes, at least 60 minutes, at least 2 hours, at least 6 hours, at least 12 hours, at least 18 hours etc. The cells may be cultured in the presence of the PKC activator for a duration of from about 30 minutes to about 5 days. For example, the cells may be cultured in the presence of the PKC activator for a maximum duration of e.g. about 5 days, about 4 days, about 2 days, about 24 hours.

As would be clear to a person of skill in the art, typically, when cells are cultured for a duration of at least two days, it is beneficial to passage the cells into fresh medium. As used herein, a “passage” refers to the step of harvesting grown cells from one “parent” cell culture aliquot and reseeding them to generate a new “daughter” cell culture aliquot. Accordingly, passaging refers to the transfer of a proportion of cell suspension and/or supernatant from an aliquot to another.

When adherent cells are passaged, the cells are typically washed in PBS while still adherent, detached from the aliquot and then resuspended in media. A proportion of the resuspended cells are transferred to a new aliquot. When non-adherent cells are passaged, the cells are in suspension so a proportion of an aliquot can be directly transferred to a new aliquot.

The passage number of a cell culture refers to the number of times it has been harvested and reseeded. During passage, a volume of the parent cell culture aliquot is harvested and re-seeded in the new daughter aliquot (typically into fresh cell culture medium). In examples where the cells are cultured in the presence of the PKC activator for durations of time which include passaging, it is clear that the fresh medium used for passaging also comprises the PKC activator of interest. In other words, the cell culture medium comprising the PKC activator may be refreshed (partially or completely removed from the cells and replaced with fresh culture medium comprising the PKC activator) during cell culture.

In one non-limiting example, the PKC activator present in the cell culture medium is prostratin, an analogue, derivative or pharmaceutically acceptable salt thereof. The term “prostratin” is generally used broadly herein, to encompass analogues, derivatives or pharmaceutically acceptable salts thereof. Accordingly, throughout the description, the term “prostratin” is interchangeable with the phrase “prostratin, an analogue, derivative or pharmaceutically acceptable salt thereof”.

Prostratin may be present in the cell culture medium at any suitable concentration. For example, prostratin may be present in the cell culture medium at a concentration of at least about 0.1 μM. In one example, prostratin may be present in the cell culture medium at a concentration of at least about 0.5 μM. In other words, prostratin may be present in the cell culture medium at a concentration of at least about 1 μM, at least about 2 μM, at least about 4 μM, at least about 8 μM, at least about 10 μM, at least about 15 μM, at least about 16 μM, at least about 20 μM, at least about 25 μM, at least about 30 μM etc.

For example, prostratin may be present in the cell culture medium at a concentration between about 0.1 μM and 50 μM. In other words, prostratin may be present within the cell culture medium at a concentration of from about 0.5 μM to about 32 μM, from about 1 μM to about 32 μM, from about 2 μM to about 32 μM, from about 4 μM to about 32 μM, from about 5 μM to about 32 μM, from about 8 μM to about 32 μM, from about 10 μM to about 32 μM, from about 15 μM to about 32 μM, from about 16 μM to about 32 μM, from about 20 μM to about 32 μM, from about 25 μM to about 32 μM etc.

The cells may be cultured in the presence of prostratin for an appropriate duration of time. Typically, cells are cultured in the presence of prostratin for a duration of at least 30 minutes. In other words, the cells may be cultured in the presence of prostratin for a duration of at least 30 minutes, at least 60 minutes, at least 2 hours, at least 6 hours, at least 12 hours, at least 18 hours etc. The cells may be cultured in the presence of prostratin for a duration of from about 30 minutes to about 5 days. For example, the maximum duration may be e.g. about 5 days, about 4 days, about 2 days, about 24 hours.

For example, the cells may be cultured in the presence of at least 0.1 μM prostratin for a duration of at least 30 minutes. In other words, the cells may be cultured in the presence of at least 0.1 μM prostratin for a duration of at least 30 minutes, at least 60 minutes, at least 2 hours, at least 6 hours, at least 12 hours, at least 18 hours etc. The cells may be cultured in the presence of at least 0.1 μM prostratin for a duration of from about 30 minutes to about 5 days. For example, the maximum duration may be e.g. about 5 days, about 4 days, about 2 days, about 24 hours.

For example, the cells may be cultured in the presence of at least 0.5 μM prostratin for a duration of at least 30 minutes. In other words, the cells may be cultured in the presence of at least 0.5 μM prostratin for a duration of at least 30 minutes, at least 60 minutes, at least 2 hours, at least 6 hours, at least 12 hours, at least 18 hours etc. The cells may be cultured in the presence of at least 0.5 μM prostratin for a duration of from about 30 minutes to about 5 days. For example, the maximum duration may be e.g. about 5 days, about 4 days, about 2 days, about 24 hours.

For example, the cells may be cultured in the presence of at least 1 μM prostratin for a duration of at least 30 minutes. In other words, the cells may be cultured in the presence of at least 1 μM prostratin for a duration of at least 30 minutes, at least 60 minutes, at least 2 hours, at least 6 hours, at least 12 hours, at least 18 hours etc. The cells may be cultured in the presence of at least 1 μM prostratin for a duration of from about 30 minutes to about 5 days. For example, the maximum duration may be e.g. about 5 days, about 4 days, about 2 days, about 24 hours.

For example, the cells may be cultured in the presence of at least 2 μM prostratin for a duration of at least 30 minutes. In other words, the cells may be cultured in the presence of at least 2 μM prostratin for a duration of at least 30 minutes, at least 60 minutes, at least 2 hours, at least 6 hours, at least 12 hours, at least 18 hours etc. The cells may be cultured in the presence of at least 2 μM prostratin for a duration of from about 30 minutes to about 5 days. For example, the maximum duration may be e.g. about 5 days, about 4 days, about 2 days, about 24 hours.

For example, the cells may be cultured in the presence of at least 4 μM prostratin for a duration of at least 30 minutes. In other words, the cells may be cultured in the presence of at least 4 μM prostratin for a duration of at least 30 minutes, at least 60 minutes, at least 2 hours, at least 6 hours, at least 12 hours, at least 18 hours etc. The cells may be cultured in the presence of at least 4 μM prostratin for a duration of from about 30 minutes to about 5 days. For example, the maximum duration may be e.g. about 5 days, about 4 days, about 2 days, about 24 hours.

For example, the cells may be cultured in the presence of at least 8 μM prostratin for a duration of at least 30 minutes. In other words, the cells may be cultured in the presence of at least 8 μM prostratin for a duration of at least 30 minutes, at least 60 minutes, at least 2 hours, at least 6 hours, at least 12 hours, at least 18 hours etc. The cells may be cultured in the presence of at least 8 μM prostratin for a duration of from about 30 minutes to about 5 days. For example, the maximum duration may be e.g. about 5 days, about 4 days, about 2 days, about 24 hours.

For example, the cells may be cultured in the presence of at least 16 μM prostratin for a duration of at least 30 minutes. In other words, the cells may be cultured in the presence of at least 16 μM prostratin for a duration of at least 30 minutes, at least 60 minutes, at least 2 hours, at least 6 hours, at least 12 hours, at least 18 hours etc. The cells may be cultured in the presence of at least 16 μM prostratin for a duration of from about 30 minutes to about 5 days. For example, the maximum duration may be e.g. about 5 days, about 4 days, about 2 days, about 24 hours.

In another non-limiting example, the PKC activator present in the cell culture medium is phorbol 12-myristate 13-acetate, an analogue, derivative or pharmaceutically acceptable salt thereof. The term “phorbol 12-myristate 13-acetate” is generally used broadly herein, to encompass analogues, derivatives or pharmaceutically acceptable salts thereof. Accordingly, throughout the description, the term “phorbol 12-myristate 13-acetate” is interchangeable with the phrase “phorbol 12-myristate 13-acetate, an analogue, derivative or pharmaceutically acceptable salt thereof”.

Phorbol 12-myristate 13-acetate may be present in the cell culture medium at any suitable concentration. For example, phorbol 12-myristate 13-acetate may be present in the cell culture medium at a concentration of at least about 0.1 nM. In one example, phorbol 12-myristate 13-acetate may be present in the cell culture medium at a concentration of at least about 0.5 nM. In other words, phorbol 12-myristate 13-acetate may be present in the cell culture medium at a concentration of at least about 1 nM, at least about 2 nM, at least about 4 nM, at least about 8 nM, at least about 10 nM, at least about 15 nM, at least about 16 nM, at least about 20 nM, at least about 25 nM, at least about 30 nM etc.

For example, phorbol 12-myristate 13-acetate may be present in the cell culture medium at a concentration between about 0.1 nM and 50 nM. In other words, phorbol 12-myristate 13-acetate may be present within the cell culture medium at a concentration of from about 0.5 nM to about 32 nM, from about 1 nM to about 32 nM, from about 2 nM to about 32 nM, from about 4 nM to about 32 nM, from about 5 nM to about 32 nM, from about 8 nM to about 32 nM, from about 10 nM to about 32 nM, from about 15 nM to about 32 nM, from about 16 nM to about 32 nM, from about 20 nM to about 32 nM, from about 25 nM to about 32 nM etc.

The cells may be cultured in the presence of phorbol 12-myristate 13-acetate for an appropriate duration of time. Typically, cells are cultured in the presence of phorbol 12-myristate 13-acetate for a duration of at least 30 minutes. In other words, the cells may be cultured in the presence of phorbol 12-myristate 13-acetate for a duration of at least 30 minutes, at least 60 minutes, at least 2 hours, at least 6 hours, at least 12 hours, at least 18 hours etc. The cells may be cultured in the presence of phorbol 12-myristate 13-acetate for a duration of from about 30 minutes to about 5 days. For example, the maximum duration may be e.g. about 5 days, about 4 days, about 2 days, about 24 hours.

For example, the cells may be cultured in the presence of at least 0.1 nM phorbol 12-myristate 13-acetate for a duration of at least 30 minutes. In other words, the cells may be cultured in the presence of at least 0.1 nM phorbol 12-myristate 13-acetate for a duration of at least 30 minutes, at least 60 minutes, at least 2 hours, at least 6 hours, at least 12 hours, at least 18 hours etc. The cells may be cultured in the presence of at least 0.1 nM phorbol 12-myristate 13-acetate for a duration of from about 30 minutes to about 5 days. For example, the maximum duration may be e.g. about 5 days, about 4 days, about 2 days, about 24 hours.

For example, the cells may be cultured in the presence of at least 0.5 nM phorbol 12-myristate 13-acetate for a duration of at least 30 minutes. In other words, the cells may be cultured in the presence of at least 0.5 nM phorbol 12-myristate 13-acetate for a duration of at least 30 minutes, at least 60 minutes, at least 2 hours, at least 6 hours, at least 12 hours, at least 18 hours etc. The cells may be cultured in the presence of at least 0.5 nM phorbol 12-myristate 13-acetate for a duration of from about 30 minutes to about 5 days. For example, the maximum duration may be e.g. about 5 days, about 4 days, about 2 days, about 24 hours.

For example, the cells may be cultured in the presence of at least 1 nM phorbol 12-myristate 13-acetate for a duration of at least 30 minutes. In other words, the cells may be cultured in the presence of at least 1 nM phorbol 12-myristate 13-acetate for a duration of at least 30 minutes, at least 60 minutes, at least 2 hours, at least 6 hours, at least 12 hours, at least 18 hours etc. The cells may be cultured in the presence of at least 1 nM phorbol 12-myristate 13-acetate for a duration of from about 30 minutes to about 5 days. For example, the maximum duration may be e.g. about 5 days, about 4 days, about 2 days, about 24 hours.

For example, the cells may be cultured in the presence of at least 2 nM phorbol 12-myristate 13-acetate for a duration of at least 30 minutes. In other words, the cells may be cultured in the presence of at least 2 nM phorbol 12-myristate 13-acetate for a duration of at least 30 minutes, at least 60 minutes, at least 2 hours, at least 6 hours, at least 12 hours, at least 18 hours etc. The cells may be cultured in the presence of at least 2 nM phorbol 12-myristate 13-acetate for a duration of from a about 30 minutes to about 5 days. For example, the maximum duration may be e.g. about 5 days, about 4 days, about 2 days, about 24 hours.

For example, the cells may be cultured in the presence of at least 4 nM phorbol 12-myristate 13-acetate for a duration of at least 30 minutes. In other words, the cells may be cultured in the presence of at least 4 nM phorbol 12-myristate 13-acetate for a duration of at least 30 minutes, at least 60 minutes, at least 2 hours, at least 6 hours, at least 12 hours, at least 18 hours etc. The cells may be cultured in the presence of at least 4 nM phorbol 12-myristate 13-acetate for a duration of from about 30 minutes to about 5 days. For example, the maximum duration may be e.g. about 5 days, about 4 days, about 2 days, about 24 hours.

For example, the cells may be cultured in the presence of at least 8 nM phorbol 12-myristate 13-acetate for a duration of at least 30 minutes. In other words, the cells may be cultured in the presence of at least 8 nM phorbol 12-myristate 13-acetate for a duration of at least 30 minutes, at least 60 minutes, at least 2 hours, at least 6 hours, at least 12 hours, at least 18 hours etc. The cells may be cultured in the presence of at least 8 nM phorbol 12-myristate 13-acetate for a duration of from about 30 minutes to about 5 days. For example, the maximum duration may be e.g. about 5 days, about 4 days, about 2 days, about 24 hours.

For example, the cells may be cultured in the presence of at least 16 nM phorbol 12-myristate 13-acetate for a duration of at least 30 minutes. In other words, the cells may be cultured in the presence of at least 16 nM phorbol 12-myristate 13-acetate for a duration of at least 30 minutes, at least 60 minutes, at least 2 hours, at least 6 hours, at least 12 hours, at least 18 hours etc. The cells may be cultured in the presence of at least 16 nM phorbol 12-myristate 13-acetate for a duration of from about 30 minutes to about 5 days. For example, the maximum duration may be e.g. about 5 days, about 4 days, about 2 days, about 24 hours.

The PKC activator may be included in the cell culture medium via any appropriate means. For example, the PKC activator may be added to the cell culture medium as a supplement. In this example, the PKC activator may be added to the cell culture medium before or after the cell culture medium has been added to the cells. The PKC activator may also be included in the cell culture through other means known in the art.

The presence of a PKC activator in the cell culture medium during viral vector production has been shown to increase viral vector titre. In this context, an “increase in viral vector titre” may include “inducing viral vector titre” or “enhancing viral vector titre” during viral vector production. As would be clear to a person of skill in the art, in this context, “increasing” viral vector titre refers to an increase in viral vector titre relative to viral vector production in the absence of the PKC activator. Thus, production of a viral vector in the presence of a PKC activator increases viral vector titre relative to viral vector production in the absence of the PKC activator. A suitable assay for the measurement of viral vector titre is as described herein (e.g. for a lentivirus). In some embodiments, the increase in viral vector titre (e.g. lentiviral vector titre) occurs in the presence or absence of a functional 5′LTR polyA site. In some embodiments, the increase in viral vector titres (e.g. lentiviral vector titre) mediated by a PKC activator is independent of polyA site suppression in the 5′LTR of the vector genome.

In some examples, the presence of a PKC activator may increase viral vector titre during viral vector production by at least 30% relative to viral vector production in the absence of the PKC activator. Suitably, PKC activator may increase viral vector titre during viral vector production by at least 35% (suitably at least 40%, 45%, 50%, 60%, 70%, 100%, 150%, 200%, 250%, 300%, 350%, 400%, 450%, 500%, 550%, 600%, 650%, 700%, 750%, 800%, 850%, 900%, 950% or 1000%) relative to viral vector production in the absence of the PKC activator.

The methods described herein are particularly advantageous when viral vector production occurs in the presence of a PKC activator and a HDAC inhibitor. Accordingly, a method for producing a viral vector is provided, the method comprising culturing a cell comprising nucleic acid sequences encoding viral vector components in a cell culture medium that comprises a PKC activator and a HDAC inhibitor.

Methods for inducing viral vector production in which cells are cultured in the presence of a HDAC inhibitor (commonly sodium butyrate) in the absence of other transcription promoting agents are known. The inventors have now found that exposing cells to the specific combination of a HDAC inhibitor and a PKC activator resulted in an unexpected further increase (enhancement) in viral vector titre during viral vector production.

The combination of a PKC activator and a HDAC inhibitor described herein may be useful for the production of any suitable viral vector. Examples of viral vectors that may be produced by the methods are provided elsewhere herein and include a viral vector selected from the group consisting of: a retroviral vector, an adenoviral vector, an adeno-associated viral vector, a herpes simplex viral vector and a vaccinia viral vector. Details of each of these vectors is provided elsewhere and applies equally here.

Methods wherein a combination of a PKC activator and a HDAC inhibitor are used are particularly suitable for the production of a retroviral vector, particularly for lentiviral vector production. For example, the methods described herein may be used for the production of a lentiviral vector selected from the group consisting of: HIV-1, HIV-2, SIV, FIV, BIV, EIAV, CAEV and visna lentiviral vector. In one example, the methods described herein may be used for the production of a lentiviral vector selected from an HIV (e.g. HIV-1, HIV-2) or an EIAV lentiviral vector.

The methods provided herein wherein a combination of a PKC activator and a HDAC inhibitor are used are particularly useful when producing self-inactivating (SIN) viral vectors (for example, SIN lentiviral vectors). The characteristics for SIN vectors are described in more detail elsewhere herein. In a particular example, the SIN vector may be a 3rd generation SIN viral vector (e.g. a 3rd generation lentiviral vector).

In one example, the cell culture medium comprises a PKC activator and a HDAC inhibitor.

Nuclear DNA is wrapped around histones. Modification of histones by acetylation plays a key role in epigenetic regulation of gene expression and is controlled by the balance between the activity of histone acetyltransferases (HAT) and histone deacetylases (HDAC) which attach or remove the acetyl group, respectively, from the lysine tails of these histone barrels. Acetyl groups mask positive lysine residues from interacting closely with the DNA phosphate-backbone, resulting in a more “open” chromatin state. HDACs remove these acetyl groups, resulting in a more “closed” or compacted DNA-histone state.

Histone deacetylases (HDACs) are enzymes that remove acetyl groups from the lysine residues in core histones, thus leading to the formation of a condensed and transcriptionally silenced chromatin. There are currently 18 known histone deacetylases, which are classified into four groups. Class I HDACs, which include HDAC1, HDAC2, HDAC3, and HDAC8, are related to the yeast RPD3 gene. Class II HDACs, which include HDAC4, HDAC5, HDAC6, HDAC7, HDAC9, and HDAC10, are related to the yeast Hda1 gene. Class III HDACs, which are also known as the sirtuins are related to the Sir2 gene and include SIRT1-7. Class IV HDACs, which contains only HDAC11, has features of both Class I and II HDACs.

The term “HDAC” as used herein, refers to one or more histone deacetylase.

The term “HDAC inhibitor” as used herein, refers to a substance that reduces the rate of reaction catalyzed by HDAC. Exemplary HDAC inhibitors are disclosed in Xu et al, Oncogene (2007), 26, 5541-5552, in particular in Table 2 on page 5543, the entire content of which is incorporated by reference herein. The terms “histone deacetylase inhibitor”, “HDAC inhibitor” and “HDACi” are used interchangeably herein. HDAC inhibitors described herein may be selective or non-selective to a particular type of histone deacetylase enzyme.

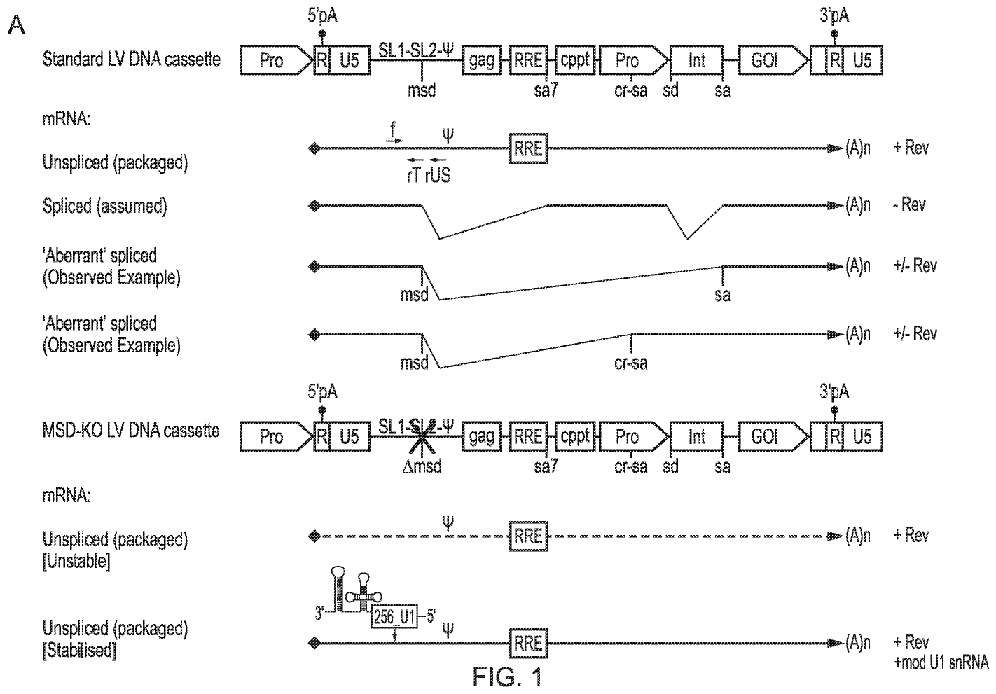

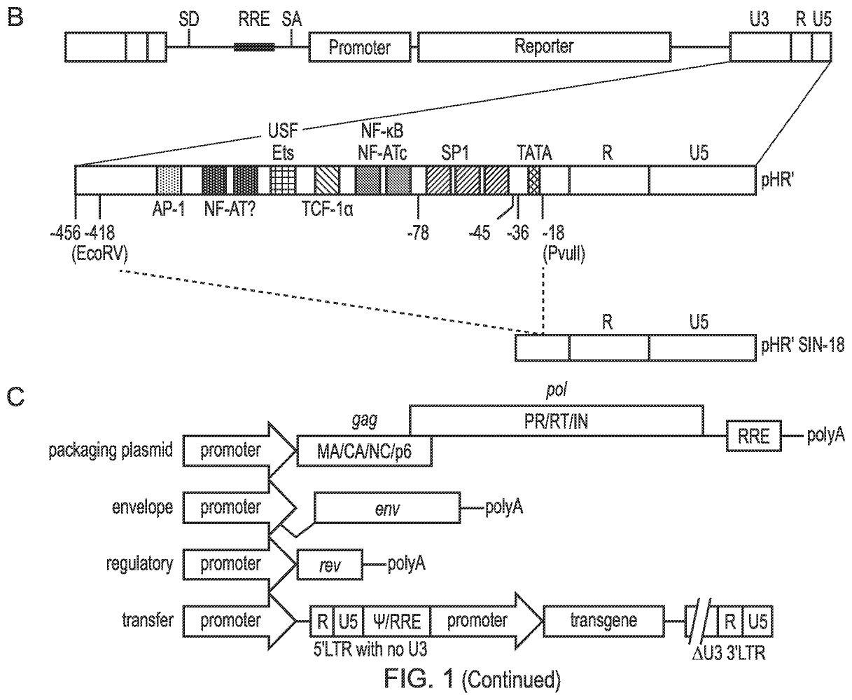

Several HDAC inhibitors are known in the art. A HDAC inhibitor may be selected from a hydroxamate, a cyclic peptide, a benzamide, or an aliphatic acid, or a pharmaceutically acceptable salt or derivative thereof, Examples of HDAC inhibitors that fall within each of these classes can be found in FIG. 1 of Kim H J, Bae S C. Histone deacetylase inhibitors: molecular mechanisms of action and clinical trials as anti-cancer drugs. Am J Transl Res. 2011; 3(2):166-179 which is incorporated herein in its entirety. The HDAC inhibitor may be an aliphatic add, such as butyric acid, valproic add, valeric add, or phenylbutyric acid, or a pharmaceutically acceptable salt thereof. The HDAC inhibitor may be a hydroxamate, such as suberanilohydroxarnic acid, panobinostat, belinostat, givinostat, or abexinostat, or a pharmaceutically acceptable salt thereof. The HDAC inhibitor may be a cyclic peptide, such as romidepsin, or a pharmaceutically acceptable salt thereof. The HDAC inhibitor may be a benzamide, such as pyridin-3-ylmethyl N-[[4-[(2 aminophenyl)carbamoyl]phenyl]methyl]carbamate, or N-(2-Aminophenyl)-4-[[(4-pyridin-3-ylpyrimidin-2-yl)amino]methyl]benzamide, or a pharmaceutically acceptable salt thereof. The HDAC inhibitor may be butyric acid, valproic add, valeric acid, phenylbutyric add, or suberanilohydroxarnic add, or a pharmaceutically acceptable salt thereof. Methods of identifying HDAC inhibitors are well known in the art. Examples of appropriate methods for identifying HDAC inhibitors are provided by Wei et al., PLoS Pathog. 2014 Apr. 10; 10(4):e1004071 and Zaikos et al., J Virol. 2018 Mar. 15; 92(6): e02110-17.

In one example, the HDAC inhibitor may be selected from an aliphatic HDAC inhibitor or a hydroxamic acid HDAC inhibitor. Suitable aliphatic HDAC inhibitors include but are not limited to sodium butyrate, sodium valproate or valeric acid, an analogue, derivative or pharmaceutically acceptable salt thereof. A particularly suitable aliphatic HDAC inhibitor is sodium butyrate, an analogue, derivative or pharmaceutically acceptable salt thereof.

Sodium butyrate is a sodium salt of butyrate. Sodium valproate is a sodium salt of valproic acid. Valeric acid is also known as pentanoic acid.

The term “sodium butyrate” is generally used broadly herein, to encompass analogues, derivatives or pharmaceutically acceptable salts thereof. Accordingly, throughout the description, the term “sodium butyrate” is interchangeable with the phrase “sodium butyrate, an analogue, derivative or pharmaceutically acceptable salt thereof”.

The term “sodium valproate” is generally used broadly herein, to encompass analogues, derivatives or pharmaceutically acceptable salts thereof. Accordingly, throughout the description, the term “sodium valproate” is interchangeable with the phrase “sodium valproate, an analogue, derivative or pharmaceutically acceptable salt thereof”.

The term “valeric acid” is used generally broadly herein, to encompass analogues, derivatives or pharmaceutically acceptable salts thereof. Accordingly, throughout the description, the term “valeric acid” is interchangeable with the phrase “valeric acid, an analogue, derivative or pharmaceutically acceptable salt thereof”.

Suitable hydroxamic acid HDAC inhibitors include, but are not limited to suberanilohydroxamic acid, an analogue, derivative or pharmaceutically acceptable salt thereof.

The term “suberanilohydroxamic acid” is generally used broadly herein, to encompass analogues, derivatives or pharmaceutically acceptable salts thereof. Accordingly, throughout the description, the term “suberanilohydroxamic acid” is interchangeable with the phrase “suberanilohydroxamic acid, an analogue, derivative or pharmaceutically acceptable salt thereof”.

In one particular example, a method for producing a viral vector is provided, the method comprising culturing a cell comprising nucleic acid sequences encoding viral vector components in a cell culture medium that comprises a PKC activator (preferably prostratin) and a HDAC inhibitor (preferably sodium butyrate). Appropriate concentrations for the PKC activator are provided above. Corresponding concentrations for the HDAC inhibitor are provided below.

The HDAC inhibitor may be present in the cell culture medium at any suitable concentration. A range of suitable concentrations may readily be identified by a person of skill in the art, using routine experimentation. For example, methods similar to those in the examples section below may be used to identify a HDAC inhibitor concentration that increases viral titre. Several methods for measuring viral titre are known in the art. Further details are provided elsewhere herein.

The cells may be cultured in the presence of the HDAC inhibitor for an appropriate duration of time. Typically, the PKC activator and HDAC inhibitor are present in the culture medium at the same time for at least some of the culture time. The PKC activator and HDAC inhibitor may be added to the cells simultaneously or sequentially. For example, the HDAC inhibitor may be added to the cells, with the PKC activator being added to the cells at the same time or at some point after the HDAC inhibitor. The PKC activator may be added to the cells 0 to 10 hours after the HDAC inhibitor for example.

Typically, cells are cultured in the presence of the HDAC inhibitor for a similar duration as the PKC activator they are used in combination with. For example, the cells may be cultured in the presence of a HDAC inhibitor for a duration of at least 30 minutes. In other words, the cells may be cultured in the presence of the HDAC inhibitor for a duration of at least 30 minutes, at least 60 minutes, at least 2 hours, at least 6 hours, at least 12 hours, at least 18 hours etc. The cells may be cultured in the presence of the HDAC inhibitor for a duration of from about 30 minutes to about 5 days. For example, the maximum duration may be e.g. about 5 days, about 4 days, about 2 days, about 24 hours.

As would be clear to a person of skill in the art, typically, when cells are cultured for a duration of at least two days, it is beneficial to passage the cells into fresh medium. In examples where the cells are cultured in the presence of the HDAC inhibitor for durations of time which include passaging, it is clear that the fresh medium used for passaging also comprises the HDAC inhibitor of interest. In other words, the cell culture medium comprising the HDAC inhibitor may be refreshed (partially or completely removed from the cells and replaced with fresh culture medium comprising the HDAC inhibitor) during culture.

For example, the HDAC inhibitor present in the cell culture medium may be sodium butyrate. Sodium butyrate may be present in the cell culture medium at any suitable concentration. For example, sodium butyrate may be present in the cell culture medium at a concentration of at least about 1 mM. In one example, sodium butyrate may be present in the cell culture medium at a concentration of at least about 2 mM. In other words, sodium butyrate may be present in the cell culture medium at a concentration of at least about 2.5 mM, at least about 3 mM, at least about 4 mM, at least about 5 mM, at least about 10 mM, at least about 15 mM, at least about 20 mM, at least about 25 mM etc.

For example, sodium butyrate may be present in the cell culture medium at a concentration between about 1 mM and 50 mM. In other words, sodium butyrate may be present within the cell culture medium at a concentration of from about 2 mM to about 30 mM, from about 2.5 mM to about 30 mM, from about 3 mM to about 30 mM, from about 4 mM to about 30 mM, from about 5 mM to about 30 mM, from about 8 mM to about 30 mM, from about 10 mM to about 30 mM, from about 15 mM to about 30 mM, from about 20 mM to about 30 mM, from about 25 mM to about 30 mM etc.

The cells may be cultured in the presence of sodium butyrate for an appropriate duration of time. Typically, cells are cultured in the presence of sodium butyrate for a similar duration as for the PKC activator that it is used in combination with. In one example, the cells are cultured in the presence of sodium butyrate for at least 30 minutes. In other words, the cells may be cultured in the presence of sodium butyrate for a duration of at least 30 minutes, at least 60 minutes, at least 2 hours, at least 6 hours, at least 12 hours, at least 18 hours etc. The cells may be cultured in the presence of sodium butyrate for a duration of from about 30 minutes to about 5 days. For example, the maximum duration may be e.g. about 5 days, about 4 days, about 2 days, about 24 hours.

For example, the cells may be cultured in the presence of at least 1 mM sodium butyrate for a duration of at least 30 minutes. In other words, the cells may be cultured in the presence of at least 1 mM sodium butyrate for a duration of at least 30 minutes, at least 60 minutes, at least 2 hours, at least 6 hours, at least 12 hours, at least 18 hours etc. The cells may be cultured in the presence of at least 1 mM sodium butyrate for a duration of from about 30 minutes to about 5 days. For example, the maximum duration may be e.g. about 5 days, about 4 days, about 2 days, about 24 hours.

For example, the cells may be cultured in the presence of at least 2 mM sodium butyrate for a duration of at least 30 minutes. In other words, the cells may be cultured in the presence of at least 2 mM sodium butyrate for a duration of at least 30 minutes, at least 60 minutes, at least 2 hours, at least 6 hours, at least 12 hours, at least 18 hours etc. The cells may be cultured in the presence of at least 2 mM sodium butyrate for a duration of from about 30 minutes to about 5 days. For example, the maximum duration may be e.g. about 5 days, about 4 days, about 2 days, about 24 hours.

For example, the cells may be cultured in the presence of at least 2.5 mM sodium butyrate for a duration of at least 30 minutes. In other words, the cells may be cultured in the presence of at least 2.5 mM sodium butyrate for a duration of at least 30 minutes, at least 60 minutes, at least 2 hours, at least 6 hours, at least 12 hours, at least 18 hours etc. The cells may be cultured in the presence of at least 2.5 mM sodium butyrate for a duration of from about 30 minutes to about 5 days. For example, the maximum duration may be e.g. about 5 days, about 4 days, about 2 days, about 24 hours.

For example, the cells may be cultured in the presence of at least 4 mM sodium butyrate for a duration of at least 30 minutes. In other words, the cells may be cultured in the presence of at least 4 mM sodium butyrate for a duration of at least 30 minutes, at least 60 minutes, at least 2 hours, at least 6 hours, at least 12 hours, at least 18 hours etc. The cells may be cultured in the presence of at least 4 mM sodium butyrate for a duration of from about 30 minutes to about 5 days. For example, the maximum duration may be e.g. about 5 days, about 4 days, about 2 days, about 24 hours.

For example, the cells may be cultured in the presence of at least 5 mM sodium butyrate for a duration of at least 30 minutes. In other words, the cells may be cultured in the presence of at least 5 mM sodium butyrate for a duration of at least 30 minutes, at least 60 minutes, at least 2 hours, at least 6 hours, at least 12 hours, at least 18 hours etc. The cells may be cultured in the presence of at least 5 mM sodium butyrate for a duration of from about 30 minutes to about 5 days. For example, the maximum duration may be e.g. about 5 days, about 4 days, about 2 days, about 24 hours.

For example, the cells may be cultured in the presence of at least 8 mM sodium butyrate for a duration of at least 30 minutes. In other words, the cells may be cultured in the presence of at least 8 mM sodium butyrate for a duration of at least 30 minutes, at least 60 minutes, at least 2 hours, at least 6 hours, at least 12 hours, at least 18 hours etc. The cells may be cultured in the presence of at least 8 mM sodium butyrate for a duration of from about 30 minutes to about 5 days. For example, the maximum duration may be e.g. about 5 days, about 4 days, about 2 days, about 24 hours.

The above concentrations and durations for sodium butyrate may be appropriately combined with the concentrations and durations provided for prostratin, for example.

Alternatively, the above concentrations and durations for sodium butyrate may be appropriately combined with the concentrations and durations provided for phorbol 12-myristate 13-acetate, for example.

As another example, the HDAC inhibitor present in the cell culture medium may be sodium valproate. Sodium valproate may be present in the cell culture medium at any suitable concentration. For example, sodium valproate may be present in the cell culture medium at a concentration of at least about 1 mM. In one example, sodium valproate may be present in the cell culture medium at a concentration of at least about 2 mM. In other words, sodium valproate may be present in the cell culture medium at a concentration of at least about 2.5 mM, at least about 3 mM, at least about 4 mM, at least about 5 mM, at least about 10 mM, at least about 15 mM, at least about 20 mM, at least about 25 mM etc.

For example, sodium valproate may be present in the cell culture medium at a concentration between about 1 mM and 50 mM. In other words, sodium valproate may be present within the cell culture medium at a concentration of from about 2 mM to about 30 mM, from about 2.5 mM to about 30 mM, from about 3 mM to about 30 mM, from about 4 mM to about 30 mM, from about 5 mM to about 30 mM, from about 8 mM to about 30 mM, from about 10 mM to about 30 mM, from about 15 mM to about 30 mM, from about 20 mM to about 30 mM, from about 25 mM to about 30 mM etc.

The cells may be cultured in the presence of sodium valproate for an appropriate duration of time. Typically, cells are cultured in the presence of sodium valproate for a similar duration as for the PKC activator that it is used in combination with. In one example, the cells are cultured in the presence of sodium valproate for at least 30 minutes. In other words, the cells may be cultured in the presence of sodium valproate for a duration of at least 30 minutes, at least 60 minutes, at least 2 hours, at least 6 hours, at least 12 hours, at least 18 hours etc.

The cells may be cultured in the presence of sodium valproate for a duration of from about 30 minutes to about 5 days. For example, the maximum duration may be e.g. about 5 days, about 4 days, about 2 days, about 24 hours.

For example, the cells may be cultured in the presence of at least 1 mM sodium valproate for a duration of at least 30 minutes. In other words, the cells may be cultured in the presence of at least 1 mM sodium valproate for a duration of at least 30 minutes, at least 60 minutes, at least 2 hours, at least 6 hours, at least 12 hours, at least 18 hours etc. The cells may be cultured in the presence of at least 1 mM sodium valproate for a duration of from about 30 minutes to about 5 days. For example, the maximum duration may be e.g. about 5 days, about 4 days, about 2 days, about 24 hours.

For example, the cells may be cultured in the presence of at least 2 mM sodium valproate for a duration of at least 30 minutes. In other words, the cells may be cultured in the presence of at least 2 mM sodium valproate for a duration of at least 30 minutes, at least 60 minutes, at least 2 hours, at least 6 hours, at least 12 hours, at least 18 hours etc. The cells may be cultured in the presence of at least 2 mM sodium valproate for a duration of from about 30 minutes to about 5 days. For example, the maximum duration may be e.g. about 5 days, about 4 days, about 2 days, about 24 hours.

For example, the cells may be cultured in the presence of at least 2.5 mM sodium valproate for a duration of at least 30 minutes. In other words, the cells may be cultured in the presence of at least 2.5 mM sodium valproate for a duration of at least 30 minutes, at least 60 minutes, at least 2 hours, at least 6 hours, at least 12 hours, at least 18 hours etc. The cells may be cultured in the presence of at least 2.5 mM sodium valproate for a duration of from about 30 minutes to about 5 days. For example, the maximum duration may be e.g. about 5 days, about 4 days, about 2 days, about 24 hours.

For example, the cells may be cultured in the presence of at least 4 mM sodium valproate for a duration of at least 30 minutes. In other words, the cells may be cultured in the presence of at least 4 mM sodium valproate for a duration of at least 30 minutes, at least 60 minutes, at least 2 hours, at least 6 hours, at least 12 hours, at least 18 hours etc. The cells may be cultured in the presence of at least 4 mM sodium valproate for a duration of from about 30 minutes to about 5 days. For example, the maximum duration may be e.g. about 5 days, about 4 days, about 2 days, about 24 hours.

For example, the cells may be cultured in the presence of at least 5 mM sodium valproate for a duration of at least 30 minutes. In other words, the cells may be cultured in the presence of at least 5 mM sodium valproate for a duration of at least 30 minutes, at least 60 minutes, at least 2 hours, at least 6 hours, at least 12 hours, at least 18 hours etc. The cells may be cultured in the presence of at least 5 mM sodium valproate for a duration of from about 30 minutes to about 5 days. For example, the maximum duration may be e.g. about 5 days, about 4 days, about 2 days, about 24 hours.

For example, the cells may be cultured in the presence of at least 8 mM sodium valproate for a duration of at least 30 minutes. In other words, the cells may be cultured in the presence of at least 8 mM sodium valproate for a duration of at least 30 minutes, at least 60 minutes, at least 2 hours, at least 6 hours, at least 12 hours, at least 18 hours etc. The cells may be cultured in the presence of at least 8 mM sodium valproate for a duration of from about 30 minutes to about 5 days. For example, the maximum duration may be e.g. about 5 days, about 4 days, about 2 days, about 24 hours.

The above concentrations and durations for sodium valproate may be appropriately combined with the concentrations and durations provided for prostratin, for example.

Alternatively, the above concentrations and durations for sodium valproate may be appropriately combined with the concentrations and durations provided for phorbol 12-myristate 13-acetate, for example.

As another example, the HDAC inhibitor present in the cell culture medium may be valeric acid. Valeric acid may be present in the cell culture medium at any suitable concentration. For example, valeric acid may be present in the cell culture medium at a concentration of at least about 1 mM. In one example, valeric acid may be present in the cell culture medium at a concentration of at least about 2 mM. In other words, valeric acid may be present in the cell culture medium at a concentration of at least about 2.5 mM, at least about 3 mM, at least about 4 mM, at least about 5 mM, at least about 10 mM, at least about 15 mM, at least about 20 mM, at least about 25 mM etc.

For example, valeric acid may be present in the cell culture medium at a concentration between about 1 mM and 50 mM. In other words, valeric acid may be present within the cell culture medium at a concentration of from about 2 mM to about 30 mM, from about 2.5 mM to about 30 mM, from about 3 mM to about 30 mM, from about 4 mM to about 30 mM, from about 5 mM to about 30 mM, from about 8 mM to about 30 mM, from about 10 mM to about 30 mM, from about 15 mM to about 30 mM, from about 20 mM to about 30 mM, from about 25 mM to about 30 mM etc.

The cells may be cultured in the presence of valeric acid for an appropriate duration of time. Typically, cells are cultured in the presence of valeric acid for a similar duration as for the PKC activator that it is used in combination with. In one example, the cells are cultured in the presence of valeric acid for at least 30 minutes. In other words, the cells may be cultured in the presence of valeric acid for a duration of at least 30 minutes, at least 60 minutes, at least 2 hours, at least 6 hours, at least 12 hours, at least 18 hours etc. The cells may be cultured in the presence of valeric acid for a duration of from about 30 minutes to about 5 days. For example, the maximum duration may be e.g. about 5 days, about 4 days, about 2 days, about 24 hours.

For example, the cells may be cultured in the presence of at least 0.1 mM valeric acid for a duration of at least 30 minutes. In other words, the cells may be cultured in the presence of at least 0.1 mM valeric acid for a duration of at least 30 minutes, at least 60 minutes, at least 2 hours, at least 6 hours, at least 12 hours, at least 18 hours etc. The cells may be cultured in the presence of at least 0.1 mM valeric acid for a duration of from about 30 minutes to about 5 days. For example, the maximum duration may be e.g. about 5 days, about 4 days, about 2 days, about 24 hours.

For example, the cells may be cultured in the presence of at least 0.5 mM valeric acid for a duration of at least 30 minutes. In other words, the cells may be cultured in the presence of at least 0.5 mM valeric acid for a duration of at least 30 minutes, at least 60 minutes, at least 2 hours, at least 6 hours, at least 12 hours, at least 18 hours etc. The cells may be cultured in the presence of at least 0.5 mM valeric acid for a duration of from about 30 minutes to about 5 days. For example, the maximum duration may be e.g. about 5 days, about 4 days, about 2 days, about 24 hours.

For example, the cells may be cultured in the presence of at least 1 mM valeric acid for a duration of at least 30 minutes. In other words, the cells may be cultured in the presence of at least 1 mM valeric acid for a duration of at least 30 minutes, at least 60 minutes, at least 2 hours, at least 6 hours, at least 12 hours, at least 18 hours etc. The cells may be cultured in the presence of at least 1 mM valeric acid for a duration of from about 30 minutes to about 5 days. For example, the maximum duration may be e.g. about 5 days, about 4 days, about 2 days, about 24 hours.

For example, the cells may be cultured in the presence of at least 2 mM valeric acid for a duration of at least 30 minutes. In other words, the cells may be cultured in the presence of at least 2 mM valeric acid for a duration of at least 30 minutes, at least 60 minutes, at least 2 hours, at least 6 hours, at least 12 hours, at least 18 hours etc. The cells may be cultured in the presence of at least 2 mM valeric acid for a duration of from about 30 minutes to about 5 days. For example, the maximum duration may be e.g. about 5 days, about 4 days, about 2 days, about 24 hours.

For example, the cells may be cultured in the presence of at least 4 mM valeric acid for a duration of at least 30 minutes. In other words, the cells may be cultured in the presence of at least 4 mM valeric acid for a duration of at least 30 minutes, at least 60 minutes, at least 2 hours, at least 6 hours, at least 12 hours, at least 18 hours etc. The cells may be cultured in the presence of at least 4 mM valeric acid for a duration of from about 30 minutes to about 5 days. For example, the maximum duration may be e.g. about 5 days, about 4 days, about 2 days, about 24 hours.

For example, the cells may be cultured in the presence of at least 8 mM valeric acid for a duration of at least 30 minutes. In other words, the cells may be cultured in the presence of at least 8 mM valeric acid for a duration of at least 30 minutes, at least 60 minutes, at least 2 hours, at least 6 hours, at least 12 hours, at least 18 hours etc. The cells may be cultured in the presence of at least 8 mM valeric acid for a duration of from about 30 minutes to about 5 days. For example, the maximum duration may be e.g. about 5 days, about 4 days, about 2 days, about 24 hours.

The above concentrations and durations for valeric acid may be appropriately combined with the concentrations and durations provided for prostratin, for example.

Alternatively, the above concentrations and durations for valeric acid may be appropriately combined with the concentrations and durations provided for phorbol 12-myristate 13-acetate, for example.

As another example, the HDAC inhibitor present in the cell culture medium may be suberanilohydroxamic acid. Suberanilohydroxamic acid may be present in the cell culture medium at any suitable concentration. For example, suberanilohydroxamic acid may be present in the cell culture medium at a concentration of at least about 0.1 μM. In one example, suberanilohydroxamic acid may be present in the cell culture medium at a concentration of at least about 0.5 μM. In other words, suberanilohydroxamic acid may be present in the cell culture medium at a concentration of at least about 1 μM, at least about 2 μM, at least about 3 μM, at least about 4 μM, at least about 5 μM, at least about 6 μM, at least about 10 μM etc.

For example, suberanilohydroxamic acid may be present in the cell culture medium at a concentration between about 0.1 μM and 50 μM. In other words, suberanilohydroxamic acid may be present within the cell culture medium at a concentration of from about 0.5 μM to about 30 μM, from about 0.5 μM to about 16 μM, from about 1 μM to about 16 μM, from about 2 μM to about 16 μM, from about 3 μM to about 16 μM, from about 4 μM to about 16 μM, from about 5 μM to about 16 μM, from about 6 μM to about 16 μM, from about 10 μM to about 16 μM, from about 10 μM to about 30 μM etc.

The cells may be cultured in the presence of suberanilohydroxamic acid for an appropriate duration of time. Typically, cells are cultured in the presence of valeric acid for a similar duration as for the PKC activator that it is used in combination with. In one example, the cells are cultured in the presence of suberanilohydroxamic acid for at least 30 minutes. In other words, the cells may be cultured in the presence of suberanilohydroxamic acid for a duration of at least 30 minutes, at least 60 minutes, at least 2 hours, at least 6 hours, at least 12 hours, at least 18 hours etc. The cells may be cultured in the presence of suberanilohydroxamic acid for a duration of from about 30 minutes to about 5 days. For example, the maximum duration may be e.g. about 5 days, about 4 days, about 2 days, about 24 hours.

For example, the cells may be cultured in the presence of at least 1 μM suberanilohydroxamic acid for a duration of at least 30 minutes. In other words, the cells may be cultured in the presence of at least 1 μM suberanilohydroxamic acid for a duration of at least 30 minutes, at least 60 minutes, at least 2 hours, at least 6 hours, at least 12 hours, at least 18 hours etc. The cells may be cultured in the presence of at least 1 μM suberanilohydroxamic acid for a duration of from about 30 minutes to about 5 days. For example, the maximum duration may be e.g. about 5 days, about 4 days, about 2 days, about 24 hours.

For example, the cells may be cultured in the presence of at least 2 μM suberanilohydroxamic acid for a duration of at least 30 minutes. In other words, the cells may be cultured in the presence of at least 2 μM suberanilohydroxamic acid for a duration of at least 30 minutes, at least 60 minutes, at least 2 hours, at least 6 hours, at least 12 hours, at least 18 hours etc. The cells may be cultured in the presence of at least 2 μM suberanilohydroxamic acid for a duration of from about 30 minutes to about 5 days. For example, the maximum duration may be e.g. about 5 days, about 4 days, about 2 days, about 24 hours.

For example, the cells may be cultured in the presence of at least 2.5 μM suberanilohydroxamic acid for a duration of at least 30 minutes. In other words, the cells may be cultured in the presence of at least 2.5 μM suberanilohydroxamic acid for a duration of at least 30 minutes, at least 60 minutes, at least 2 hours, at least 6 hours, at least 12 hours, at least 18 hours etc. The cells may be cultured in the presence of at least 2.5 μM suberanilohydroxamic acid for a duration of from about 30 minutes to about 5 days. For example, the maximum duration may be e.g. about 5 days, about 4 days, about 2 days, about 24 hours.

For example, the cells may be cultured in the presence of at least 4 μM suberanilohydroxamic acid for a duration of at least 30 minutes. In other words, the cells may be cultured in the presence of at least 4 μM suberanilohydroxamic acid for a duration of at least 30 minutes, at least 60 minutes, at least 2 hours, at least 6 hours, at least 12 hours, at least 18 hours etc. The cells may be cultured in the presence of at least 4 μM suberanilohydroxamic acid for a duration of from about 30 minutes to about 5 days. For example, the maximum duration may be e.g. about 5 days, about 4 days, about 2 days, about 24 hours.

For example, the cells may be cultured in the presence of at least 5 μM suberanilohydroxamic acid for a duration of at least 30 minutes. In other words, the cells may be cultured in the presence of at least 5 μM suberanilohydroxamic acid for a duration of at least 30 minutes, at least 60 minutes, at least 2 hours, at least 6 hours, at least 12 hours, at least 18 hours etc. The cells may be cultured in the presence of at least 5 μM suberanilohydroxamic acid for a duration of from about 30 minutes to about 5 days. For example, the maximum duration may be e.g. about 5 days, about 4 days, about 2 days, about 24 hours.

For example, the cells may be cultured in the presence of at least 8 μM suberanilohydroxamic acid for a duration of at least 30 minutes. In other words, the cells may be cultured in the presence of at least 8 μM suberanilohydroxamic acid for a duration of at least 30 minutes, at least 60 minutes, at least 2 hours, at least 6 hours, at least 12 hours, at least 18 hours etc. The cells may be cultured in the presence of at least 8 μM suberanilohydroxamic acid for a duration of from about 30 minutes to about 5 days. For example, the maximum duration may be e.g. about 5 days, about 4 days, about 2 days, about 24 hours.

The above concentrations and durations for suberanilohydroxamic acid may be appropriately combined with the concentrations and durations provided for prostratin, for example.

Alternatively, the above concentrations and durations for suberanilohydroxamic acid may be appropriately combined with the concentrations and durations provided for phorbol 12-myristate 13-acetate, for example.

The HDAC inhibitor may be included within the cell culture medium using any appropriate means. For example, the HDAC inhibitor may be added to the cell culture medium as a supplement. In this example, the HDAC inhibitor may be added to the cell culture medium before or after the cell culture medium has been added to the cells. The HDAC inhibitor may also be included in the cell culture through other means known in the art.

The presence of a HDAC inhibitor in the cell culture medium during viral vector production has been shown to increase viral vector titre when it is combined with a PKC activator as already described. In this context, an “increase in viral vector titre” may include “inducing viral vector titre” or “enhancing viral vector titre” during viral vector production. As would be clear to a person of skill in the art, in this context, “increasing” viral vector titre refers to an increase in viral vector titre relative to viral vector production in the absence of either one of the PKC activator or the HDAC inhibitor. Thus, production of a viral vector in the presence of a PKC activator and a HDAC inhibitor increases viral vector titre relative to viral vector production in the absence of either one of the PKC activator or the HDAC inhibitor. A suitable assay for the measurement of viral vector titre is as described herein (e.g. for a lentivirus). In some embodiments, the increase in viral vector titre (e.g. lentiviral vector titre) occurs in the presence or absence of a functional 5′LTR polyA site. In some embodiments, the increase of viral vector titres (e.g. lentiviral vector titre) mediated by a PKC activator is independent of polyA site suppression in the 5′LTR of the vector genome.

In some examples, the presence of a PKC activator and a HDAC inhibitor may increase viral vector titre during viral vector production by at least 30% relative to viral vector production in the absence of either one of the PKC activator or the HDAC inhibitor. Suitably, PKC activator may increase viral vector titre during viral vector production by at least 35% (suitably at least 40%, 45%, 50%, 60%, 70%, 100%, 150%, 200%, 250%, 300%, 350%, 400%, 450%, 500%, 550%, 600%, 650%, 700%, 750%, 800%, 850%, 900%, 950% or 1000%) relative to viral vector production in the absence of either one of the PKC activator or the HDAC inhibitor.

The method described herein may be part of suitable viral vector production protocol to increase viral vector titre. Accordingly, the methods provided herein may be used to produce viral vector as part of a first or subsequent (e.g. second) harvest.

The nucleotide sequences encoding vector components may be introduced into the cell either simultaneously or sequentially in any order.

As would be clear to a person of skill in the art, in these methods, the vector components may include gag, env, rev and/or the RNA genome of a lentiviral vector. These vector components are encoded by nucleotide sequences described elsewhere herein.

(ii) Viral Vector Production System

A viral vector production system is also provided herein, comprising:

i) a cell comprising nucleic acid sequences encoding viral vector components; and

ii) a cell culture medium that comprises a PKC activator.

In one example, the cell culture medium comprises a PKC activator and a HDAC inhibitor.

Details of appropriate viral vectors, PKC activators and concentrations, HDAC inhibitors and concentrations, cells and cell culture medium are provided in the methods section above and apply equally here.

Furthermore, the terms “viral vector production system”, “culture”, “cell”, “nucleic acid sequence”, “viral vector”, “viral vector components”, “cell culture” and “cell culture medium” are described in more detail in the general definitions section herein and apply equally here.

(iii) Uses

The inventors have identified, for the first time, that a PKC activator can be used for increasing viral vector titre during viral vector production. They have also shown that the PKC activator may advantageously be used in combination with a HDAC inhibitor to further increase viral vector titre during viral vector production.

Details of appropriate viral vectors, PKC activators and concentrations, HDAC inhibitors and concentrations, cells and cell culture medium are provided in the methods section above and apply equally here.

Furthermore, the terms “viral vector production system”, “culture”, “cell”, “nucleic acid sequence”, “viral vector”, “viral vector components”, “cell culture” and “cell culture medium” are described in more detail in the general definitions section herein and apply equally here.

B. Modified U1 snRNA

In the context of lentiviral vector production specifically, the methods, viral vector production systems, and uses comprising PKC activators (and optionally HDAC inhibitors) described herein may also comprise co-expression of a modified U1 snRNA as described further herein. Accordingly, when contemplating lentiviral vector production, each of the features described in relation to PKC activators (and optionally HDAC inhibitors) may be combined with the features described in this section relating to modified U1 snRNA.

The present inventors have previously shown that the output titres of lentiviral vectors can be enhanced by co-expressing non-coding RNAs based on U1 snRNAs, which have been modified so that they no longer target the endogenous sequence (a splice donor site) but now target a sequence within the vRNA molecule. They have now also found that co-expression of modified U1 snRNA during the viral vector production methods described herein results in a further increase in viral vector output titres. Accordingly, methods, systems and uses are provided herein wherein a PKC activator and a modified U1 snRNA are used in combination (optionally together with a HDAC inhibitor, as described above). This approach comprises co-expression of modified U1 snRNAs together with the other vector components during vector production. The modified U1 snRNAs are designed such that binding to the consensus splice donor site has been ablated by replacing the native splice donor annealing sequence in U1 snRNA with a heterologous sequence that is complementary to a target sequence within the vector genome vRNA. Optimal characteristics of the modified U1 snRNAs, including target sequences and complementarity length, design and modes of expression are described below.

Modified U1 snRNA

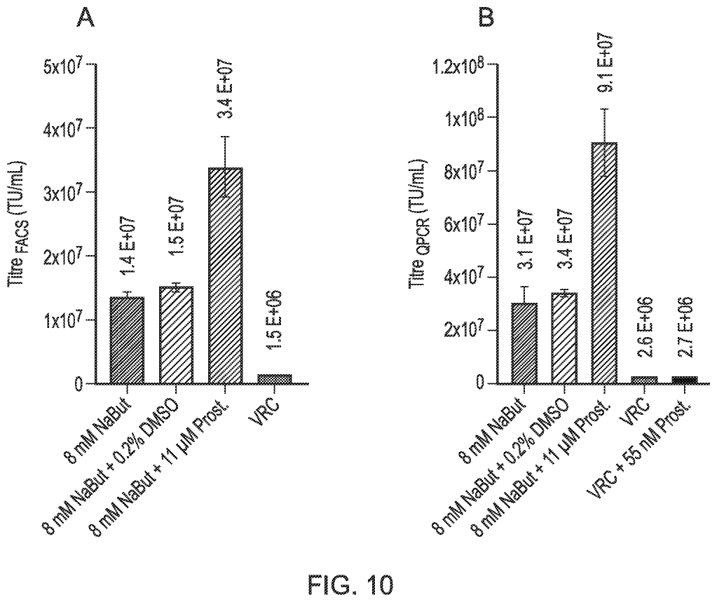

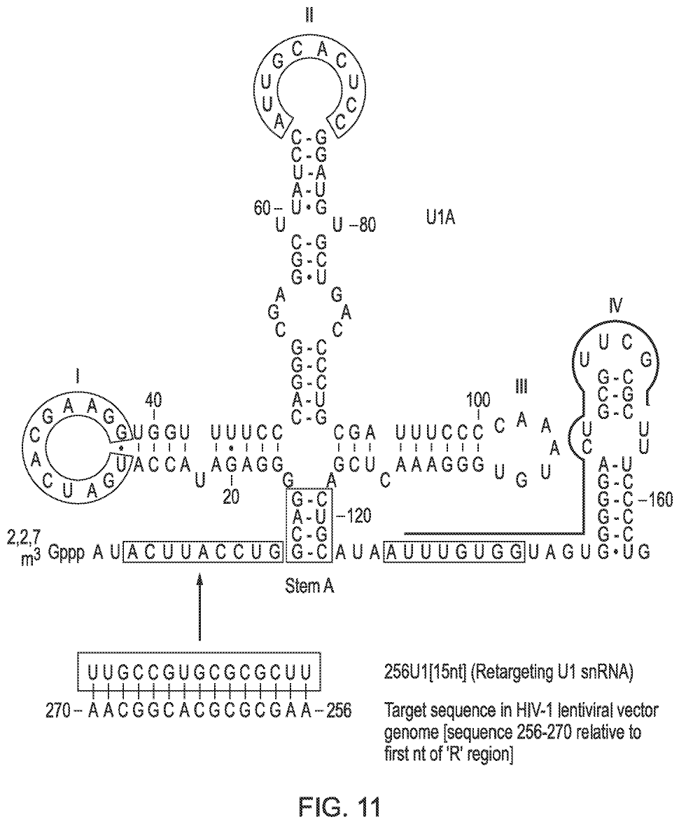

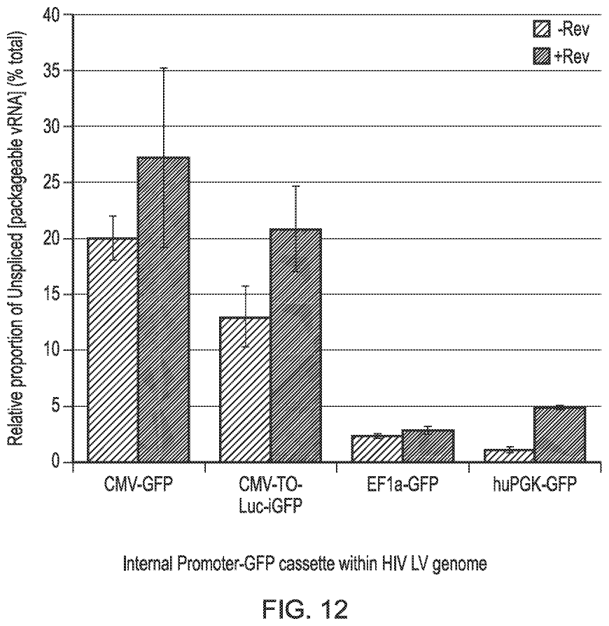

Human U1 snRNA (small nuclear RNA) is 164 nt long with a well-defined structure consisting of four stem-loops (see FIG. 11). The endogenous non-coding RNA, U1 snRNA, binds to the consensus 5′ splice donor site (e.g. 5′-MAGGURR-3′ (SEQ ID NO: 1) wherein M is A or C and R is A or G) via the native splice donor annealing sequence (e.g. 5′-ACUUACCUG-3′ (SEQ ID NO: 2)) during early steps of intron splicing. Stem loop I binds to U1A-70K protein that has been shown to be important for polyA suppression. Stem loop II binds to U1A protein, and the 5′-AUUUGUGG-3′ (SEQ ID NO: 3) sequence binds to Sm proteins, which together with Stem loop IV, is important for U1 snRNA processing. The modified U1 snRNA described herein is modified to introduce a heterologous sequence that is complementary to a target sequence within the vector genome vRNA molecule at the site of the native splice donor targeting sequence (see FIG. 11).

As used herein, the terms “modified U1 snRNA”, “re-directed U1 snRNA”, “re-targeted U1 snRNA”, “re-purposed U1 snRNA” and “mutant U1 snRNA”, mean a U1 snRNA that has been modified so that it no longer binds the consensus 5′ splice donor site sequence (e.g. 5′-MAGGURR-3′ (SEQ ID NO: 1)) that it uses to initiate the splicing process of a target gene. Thus, a modified U1 snRNA is a U1 snRNA which has been modified so that it no longer binds to the splice donor site sequence (e.g. 5′-MAGGURR-3′ (SEQ ID NO: 1)) based on complementarity of the donor site sequence with the native splice donor annealing sequence at the 5′ end of the U1 snRNA. Instead, the modified U1 snRNA is designed so that it binds a nucleotide sequence having a unique RNA sequence within the packaging region of the lentiviral vector genome molecule (target site), i.e. a sequence that is unrelated to splicing of the gene. The nucleotide sequence within the packaging region of the lentiviral vector genome molecule can be preselected. Thus, the modified U1 snRNA is a U1 snRNA which has been modified so that its 5′ end binds a nucleotide sequence within the packaging region of the lentiviral vector genome molecule. As a result, the modified U1 snRNA binds to the target site sequence based on complementarity of the target site sequence with the short sequence at the 5′ end of the modified U1 snRNA.

As used herein, the terms “native splice donor annealing sequence” and “native splice donor targeting sequence” mean the short sequence at the 5′-end of the endogenous U1 snRNA that is broadly complementary to the consensus 5′ splice donor site of introns. The native splice donor annealing sequence may be 5′-ACUUACCUG-3′ (SEQ ID NO: 2).

As used herein, the term “consensus 5′ splice donor site” means the consensus RNA sequence at the 5′ end of introns used in splice-site selection, e.g. having the sequence 5′-MAGGURR-3′ (SEQ ID NO: 1).

As used herein, the terms “nucleotide sequence within the packaging region of the lentiviral vector genome sequence”, “target sequence” and “target site” mean a site having a particular RNA sequence within the packaging region of the lentiviral vector genome molecule which has been preselected as the target site for binding the modified U1 snRNA.

As used herein, the terms “packaging region of a lentiviral vector genome molecule” and “packaging region of a lentiviral vector genome sequence” means the region at the 5′ end of a lentiviral vector genome from the beginning of the 5′ U5 domain to the terminus of the sequence derived from gag gene. Thus, the packaging region of a lentiviral vector genome molecule includes the 5′ U5 domain, PBS element, stem loop (SL) 1 element, SL2 element, SL3ψ element, SL4 element and the sequence derived from the gag gene. It is common in the art to provide the complete gag gene in trans to the genome during lentiviral vector production to enable the production of replication-defective viral vector particle. The nucleotide sequence of the gag gene provided in trans need not be encoded by wild type nucleotides but may be codon-optimised; importantly the chief attribute of the gag gene provided in trans is that it encodes and directs expression of the gag and gagpol proteins. Accordingly, it will be understood by the person skilled in the art that, if the complete gag gene is to be provided in trans during lentiviral vector production, the term “packaging region of a lentiviral vector genome molecule” may mean the region at the 5′ end of the lentiviral vector genome molecule from the beginning of the 5′ U5 domain through to the ‘core’ packaging signal at the SL3 ψ element, and the native gag nucleotide sequence from the ATG codon (present within SL4) to the end of the remaining gag nucleotide sequence present on the vector genome.

As used herein, the term “sequence derived from gag gene” means, any native sequence of the gag gene derived from the ATG codon to nucleotide 688 (Kharytonchyk, S. et. al., 2018, J. Mol. Biol., 430:2066-79) that may be present, e.g. remain, in the vector genome.

As used herein, the terms “to introduce within the first 11 nucleotides of the U1 snRNA, which encompasses the native splice donor annealing sequence, a heterologous sequence”, “to introduce within the nine nucleotides at positions 3-to-11 said heterologous sequence” and “to introduce within the first 11 nucleotides at the 5′ end of the U1 snRNA a heterologous sequence” include to replace the first 11 nucleotides, or the nine nucleotides at positions 3-to-11, of the U1 snRNA all or in part with said heterologous sequence or to modify the first 11 nucleotides, or the nine nucleotides at positons 3-to-11, of the U1 snRNA to have the same sequence as said heterologous sequence.

As used herein, the terms “to introduce within the native splice donor annealing sequence a heterologous sequence” and “to introduce within the native splice donor annealing sequence at the 5′ end of the U1 snRNA a heterologous sequence” include to replace the native splice donor annealing sequence all or in part with said heterologous sequence or to modify the native splice donor annealing sequence to have the same sequence as said heterologous sequence.

A modified U1 snRNA may be used in the methods described elsewhere herein, wherein the U1 snRNA has been modified to bind to a nucleotide sequence within the packaging region of a lentiviral vector genome sequence. In some embodiments, the modified U1 snRNA is modified at the 5′ end relative to the endogenous U1 snRNA to introduce a heterologous sequence that is complementary to a nucleotide sequence within the packaging region of a lentiviral vector genome sequence. In some embodiments, the modified U1 snRNA is modified at the 5′ end relative to the endogenous U1 snRNA to introduce within the native splice donor annealing sequence a heterologous sequence that is complementary to a nucleotide sequence within the packaging region of a lentiviral vector genome sequence.

The modified U1 snRNA may be modified at the 5′ end relative to the endogenous U1 snRNA to replace a sequence encompassing the native splice donor annealing sequence with a heterologous sequence that is complementary to a nucleotide sequence within the packaging region of a lentiviral vector genome sequence.

The modified U1 snRNA may be a modified U1 snRNA variant. The U1 snRNA variant which is modified in accordance with the invention may be a naturally occurring U1 snRNA variant, a U1 snRNA variant containing a mutation within the stem loop I region ablating U1-70K protein binding, or a U1 snRNA variant containing a mutation in the stem loop II region ablating U1A protein binding. The U1 snRNA variant containing a mutation within the stem loop I region ablating U1-70K protein binding may be U1_m1 or U1_m2, preferably U1A_m1 or U1A_m2.

In some embodiments, the modified U1 snRNA comprises a nucleotide sequence having at least 70% identity (suitably at least 75%, at least 80%, 85%, 90%, 91%, 92%, 93%, 94%, 95%, 96%, 97%, 98%, 99% identity) with the main U1 snRNA sequence [clover leaf] (nt 410-562) of the U1_256 sequence as described herein. In some embodiments, the modified U1 snRNA of the invention comprises the main U1 snRNA sequence [clover leaf] (nt 410-562) of the U1_256 sequence as described herein. The main U1 snRNA sequence [clover leaf] (nt 410-562) of the U1_256 sequence is contained in SEQ ID NO: 4:

SEQ ID NO: 4:
GCAGGGGAGATACCATGATCACGAAGGTGGTTTTCCCAGGGC
GAGGCTTATCCATTGCACTCCGGATGTGCTGACCCCTGCGAT
TTCCCCAAATGTGGGAAACTCGACTGCATAATTTGTGGTAGT
GGGGGACTGCGTTCGCGCTTTCCCCTG.

In some preferred embodiments, the first 11 nucleotides of the U1 snRNA, which encompasses the native splice donor annealing sequence, may be all or in part replaced with a heterologous sequence that is complementary to a nucleotide sequence within the packaging region of a lentiviral vector genome sequence. Suitably, 1-11 (suitably 2-11, 3-11, 1, 2, 3, 4, 5, 6, 7, 8, 9, 10 or 11), nucleic acids of the first 11 nucleotides of the U1 snRNA are replaced with a heterologous sequence that is complementary to a nucleotide sequence within the packaging region of a lentiviral vector genome sequence.

In some embodiments, the native splice donor annealing sequence, may be all or in part replaced with a heterologous sequence that is complementary to a nucleotide sequence within the packaging region of a lentiviral vector genome sequence. Suitably, 1-11 (suitably 2-11, 3-11, 5-11, 1, 2, 3, 4, 5, 6, 7, 8, 9, 10 or 11), nucleic acids of the native splice donor annealing sequence are replaced with a heterologous sequence that is complementary to a nucleotide sequence within the packaging region of a lentiviral vector genome sequence. In a preferred embodiment, the entire native splice donor annealing sequence is replaced with a heterologous sequence that is complementary to a nucleotide sequence within the packaging region of a lentiviral vector genome sequence, i.e. the native splice donor annealing sequence (e.g. 5′-ACUUACCUG-3′ (SEQ ID NO: 2)) is fully replaced with a heterologous sequence in accordance with the invention.

In some embodiments, the modified U1 snRNA comprising a heterologous sequence that is complementary to a nucleotide sequence within the packaging region of a lentiviral vector genome sequence will encode an A at the first nucleotide at the 5′ end of said heterologous sequence, irrespective of whether the A partakes in annealing to the target sequence.

In some embodiments, the modified U1 snRNA comprising a heterologous sequence that is complementary to a nucleotide sequence within the packaging region of a lentiviral vector genome sequence will encode a AU at the first two nucleotides at the 5′ end of said heterologous sequence, irrespective of whether the A or the U partakes in annealing to the target sequence.

In some embodiments, the modified U1 snRNA comprising a heterologous sequence that is complementary to a nucleotide sequence within the packaging region of a lentiviral vector genome sequence will not encode AU at the first two nucleotides at the 5′ end of said heterologous sequence, and the first nucleotide may or may not partake in annealing to the target sequence.

In some embodiments, a heterologous sequence that is complementary to a nucleotide sequence within the packaging region of a lentiviral vector genome sequence comprises at least 7 nucleotides of complementarity to said nucleotide sequence. In some embodiments, a heterologous sequence that is complementary to a nucleotide sequence within the packaging region of a lentiviral vector genome sequence comprises at least 9 nucleotides of complementarity to said nucleotide sequence. Preferably, a heterologous sequence for use in the present invention comprises 15 nucleotides of complementarity to said nucleotide sequence.

Suitably, a heterologous sequence for use in the present invention may comprise 7-25 (suitably 7-20, 7-15, 9-15, 7, 8, 9, 10, 11, 12, 13, 14, 15, 16, 17, 18, 19, 20, 21, 22, 23, 24 or 25) nucleotides. Suitably, a heterologous sequence for use in the present invention may comprise 7 nucleotides. Suitably, a heterologous sequence for use in the present invention may comprise 8 nucleotides. Suitably, a heterologous sequence for use in the present invention may comprise 9 nucleotides. Suitably, a heterologous sequence for use in the present invention may comprise 10 nucleotides. Suitably, a heterologous sequence for use in the present invention may comprise 11 nucleotides. Suitably, a heterologous sequence for use in the present invention may comprise 12 nucleotides. Suitably, a heterologous sequence for use in the present invention may comprise 13 nucleotides. Suitably, a heterologous sequence for use in the present invention may comprise 14 nucleotides.

Suitably, a heterologous sequence for use in the present invention may comprise 15 nucleotides. Suitably, a heterologous sequence for use in the present invention may comprise 16 nucleotides. Suitably, a heterologous sequence for use in the present invention may comprise 17 nucleotides. Suitably, a heterologous sequence for use in the present invention may comprise 18 nucleotides. Suitably, a heterologous sequence for use in the present invention may comprise 19 nucleotides. Suitably, a heterologous sequence for use in the present invention may comprise 20 nucleotides. Suitably, a heterologous sequence for use in the present invention may comprise 21 nucleotides. Suitably, a heterologous sequence for use in the present invention may comprise 22 nucleotides. Suitably, a heterologous sequence for use in the present invention may comprise 23 nucleotides. Suitably, a heterologous sequence for use in the present invention may comprise 24 nucleotides. Suitably, a heterologous sequence for use in the present invention may comprise 25 nucleotides.

In some embodiments, the nucleotide sequence within the packaging region of a lentiviral vector genome sequence is located within the 5′ U5 domain, PBS element, SL1 element, SL2 element, SL3ψ element, SL4 element and/or the sequence derived from gag gene. Suitably, the nucleotide sequence within the packaging region of a lentiviral vector genome sequence is located within the SL1, SL2 and/or SL3ψ element(s). In some preferred embodiments, the nucleotide sequence within the packaging region of a lentiviral vector genome sequence is located within the SL1 and/or SL2 element(s). In some particularly preferred embodiments, the nucleotide sequence within the packaging region of a lentiviral vector genome sequence is located within the SL1 element.

In some embodiments, a nucleotide sequence within the packaging region of a lentiviral vector genome sequence comprises at least 7 nucleotides. In some embodiments, a nucleotide sequence within the packaging region of a lentiviral vector genome sequence comprises at least 9 nucleotides. Suitably, a nucleotide sequence within the packaging region of a lentiviral vector genome sequence comprises 7-25 (suitably 7-20, 7-15, 9-15, 7, 8, 9, 10, 11, 12, 13, 14, or 15) nucleotides. Suitably, a nucleotide sequence within the packaging region of a lentiviral vector genome sequence comprises 7 nucleotides. Suitably, a nucleotide sequence within the packaging region of a lentiviral vector genome sequence comprises 8 nucleotides. Suitably, a nucleotide sequence within the packaging region of a lentiviral vector genome sequence comprises 9 nucleotides. Suitably, a nucleotide sequence within the packaging region of a lentiviral vector genome sequence comprises 10 nucleotides. Suitably, a nucleotide sequence within the packaging region of a lentiviral vector genome sequence comprises 11 nucleotides. Suitably, a nucleotide sequence within the packaging region of a lentiviral vector genome sequence comprises 12 nucleotides. Suitably, a nucleotide sequence within the packaging region of a lentiviral vector genome sequence comprises 13 nucleotides. Suitably, a nucleotide sequence within the packaging region of a lentiviral vector genome sequence comprises 14 nucleotides. Suitably, a nucleotide sequence within the packaging region of a lentiviral vector genome sequence comprises 15 nucleotides. Suitably, a nucleotide sequence within the packaging region of a lentiviral vector genome sequence comprises 16 nucleotides. Suitably, a nucleotide sequence within the packaging region of a lentiviral vector genome sequence comprises 17 nucleotides. Suitably, a nucleotide sequence within the packaging region of a lentiviral vector genome sequence comprises 18 nucleotides. Suitably, a nucleotide sequence within the packaging region of a lentiviral vector genome sequence comprises 19 nucleotides. Suitably, a nucleotide sequence within the packaging region of a lentiviral vector genome sequence comprises 20 nucleotides. Suitably, a nucleotide sequence within the packaging region of a lentiviral vector genome sequence comprises 21 nucleotides. Suitably, a nucleotide sequence within the packaging region of a lentiviral vector genome sequence comprises 22 nucleotides.

Suitably, a nucleotide sequence within the packaging region of a lentiviral vector genome sequence comprises 23 nucleotides. Suitably, a nucleotide sequence within the packaging region of a lentiviral vector genome sequence comprises 24 nucleotides. Suitably, a nucleotide sequence within the packaging region of a lentiviral vector genome sequence comprises 25 nucleotides. Preferably, a nucleotide sequence within the packaging region of a lentiviral vector genome sequence comprises 15 nucleotides.

The binding of a modified U1 snRNA to the nucleotide sequence within the packaging region of a lentiviral vector genome sequence may enhance lentiviral vector titre during lentiviral vector production relative to lentiviral vector production in the absence of a modified U1 snRNA.

The modified U1 snRNAs may be designed by (a) selecting a target site in the packaging region of a lentiviral vector genome for binding the modified U1 snRNA (the preselected nucleotide site); and (b) introducing within the native splice donor annealing sequence (e.g. 5′-ACUUACCUG-3′ (SEQ ID NO: 2)) at the 5′ end of the U1 snRNA a heterologous sequence that is complementary to the preselected nucleotide site selected in step (a).

The introduction of a heterologous sequence that is complementary to the target site within, or in place of, the native splice donor annealing sequence (e.g. 5′-ACUUACCUG-3′ (SEQ ID NO: 2)) at the 5′ end of the endogenous U1 snRNA using conventional techniques in molecular biology is within the capabilities of a person of ordinary skill in the art. Generally speaking, suitable routine methods include directed mutagenesis or replacement via homologous recombination.

The modification of the native splice donor annealing sequence (e.g. 5′-ACUUACCUG-3′ (SEQ ID NO: 2)) at the 5′ end of the endogenous U1 snRNA to have the same sequence as a heterologous sequence that is complementary to the target site using conventional techniques in molecular biology is within the capabilities of a person of ordinary skill in the art. For example, suitable methods include directed mutagenesis or random mutagenesis followed by selection for mutations which provide a modified U1 snRNA in accordance with the invention.

The modified U1 snRNAs of the present invention can be manufactured according to methods generally known in the art. For example, the modified U1 snRNAs can be manufactured by chemical synthesis or recombinant DNA/RNA technology.

The introduction of a nucleotide sequence encoding a modified U1 snRNA of the present invention into a cell using conventional molecular and cell biology techniques is within the capabilities of a person of ordinary skill in the art. For example, an expression cassette could be used as described below.

Lentiviral vector production may involve co-expression of a modified U1 snRNA of the invention with vector components in a suitable production cell as described herein. The production cell may be a stable production cell comprising a nucleic acid sequence encoding the modified U1 snRNA. Alternatively, the cell may be transiently transfected with a nucleic acid sequence encoding the modified U1 snRNA.

A method for producing a lentiviral vector is therefore provided, comprising the steps of:

a) introducing nucleotide sequences encoding vector components and at least one nucleotide sequence encoding a modified U1 snRNA, into a cell;

b) selecting for a cell which comprises said nucleotide sequences encoding vector components and at least one nucleotide sequence encoding a modified U1 snRNA of the invention;

c) further culturing the cell in the presence of a PKC activator (and optionally a HDAC inhibitor) under conditions in which the lentiviral vector is produced; and

d) optionally isolating the lentiviral vector.

Details of the PKC activator (and optionally a HDAC inhibitor) are provided elsewhere herein and apply equally here.

In these methods, the vector components may include gag, env, rev and/or the RNA genome of the lentiviral vector. These vector components are encoded by nucleotide sequences described elsewhere herein.

The nucleotide sequences encoding vector components and at least one nucleotide sequence encoding a modified U1 snRNA of the invention may be introduced into the cell either simultaneously or sequentially in any order. The nucleotide sequences encoding vector components may be introduced into the cell prior to at least one nucleotide sequence encoding a modified U1 snRNA of the invention. The at least one nucleotide sequence encoding a modified U1 snRNA of the invention may be introduced into the cell prior to nucleotide sequences encoding vector components.

Accordingly, the methods, systems and uses described herein comprising a PKC activator (and optionally a HDAC inhibitor) may also comprise a modified U1 snRNA, wherein said modified U1 snRNA has been modified to bind to a nucleotide sequence within the packaging region of a lentiviral vector genome sequence.

Suitably, the modified U1 snRNA may be modified to introduce a heterologous sequence that is complementary to a nucleotide sequence within the packaging region of a lentiviral vector genome sequence.

Suitably, the modified U1 snRNA may be modified at the 5′ end to introduce within the nine nucleotides at positions 3-to-11 said heterologous sequence.

Suitably, the modified U1 snRNA may be modified at the 5′ end to introduce within the native splice donor annealing sequence said heterologous sequence. Optionally, 1-9 nucleic acids of said native splice donor annealing sequence are replaced with said heterologous sequence.

Suitably, the modified U1 snRNA may be modified at the 5′ end to replace a sequence encompassing the native splice donor annealing sequence with a heterologous sequence that is complementary to a nucleotide sequence within the packaging region of a lentiviral vector genome sequence.

Suitably, the heterologous sequence may comprise at least 9 nucleotides of complementarity to a nucleotide sequence within the packaging region of a lentiviral vector genome sequence.

Suitably, the heterologous sequence may comprise 15 nucleotides of complementarity to a nucleotide sequence within the packaging region of a lentiviral vector genome sequence.

Suitably, the packaging region of a lentiviral vector genome sequence may be the beginning of the 5′ U5-domain to the terminus of the sequence derived from gag gene.

Suitably, the nucleotide sequence within the packaging region of a lentiviral vector genome sequence may be located within the 5′ U5 domain, PBS element, SL1 element, SL2 element, SL3ψ element, SL4 element and/or the sequence derived from gag gene. Suitably, the nucleotide sequence may be located within the SL1, SL2 and/or SL3ψ element(s). Suitably, the nucleotide sequence may be located within the SL1 and/or SL2 element(s). Suitably, the nucleotide sequence may be located within the SL1 element.

Suitably, the modified U1 snRNA may be a modified U1A snRNA or a modified U1A snRNA variant.

Suitably, the first two nucleotides at the 5′ end of the modified U1 snRNA are not AU.

The modified U1 snRNA may be encoded by an expression cassette.

The modified U1 snRNA may be present within a cell. In other words, a cell for producing lentiviral vectors comprising nucleotide sequences encoding viral vector components (e.g. including gag, env, rev and the RNA genome of a lentiviral vector) and at least one nucleotide sequence encoding a modified U1 snRNA as described herein may be used in the methods, systems or uses of the invention. Alternatively, a stable or transient production cell for producing lentiviral vectors may be used in the methods, systems or uses of the invention, comprising at least one nucleotide sequence encoding a modified U1 snRNA as described herein.

For example, a suitable method for producing a lentiviral vector may comprise the steps of:

a. introducing nucleotide sequences encoding vector components (e.g. including gag, env, rev and the RNA genome of a lentiviral vector), and at least one nucleotide sequence encoding a modified U1 snRNA described herein into a cell;

b. optionally selecting for a cell which comprises said nucleotide sequences encoding vector components and at least one modified U1 snRNA;

c. culturing the cell in the presence of a PKC activator (and optionally a HDAC inhibitor) under conditions in which said vector components are co-expressed with said modified U1 snRNA and the lentiviral vector is produced.

Examples of suitable modified U1 snRNA sequences are provided in Table 8 herein. This includes for example, the sequences relevant to 305U1, 179U1 and 256U1, which are used in the examples section below to illustrate the invention. Of these modified U1 snRNAs, 256U1 is particularly preferred.

Details of the PKC activator (and optionally a HDAC inhibitor) are provided elsewhere herein and apply equally here.

C. Major Splice Donor (MSD) Mutations

In the context of lentiviral vector production specifically, the methods, viral vector production systems, and uses comprising PKC activators (and optionally HDAC inhibitors and/or modified U1 snRNA) described herein may be used with a lentiviral vector genome molecule comprising an MSD mutant as described further herein. Each of the features described herein in relation to PKC activators (and optionally HDAC inhibitors and/or modified U1 snRNA) may therefore be combined with the features described in this section relating to MSD mutations.

Mutation of the major splice donor site in the packaging region of the RNA genome of a viral vector has been shown to be detrimental to vector production titres, and additionally activate a cryptic splice donor (crSD) immediately adjacent to the MSD. Aberrant splicing from the MSD or CrSD leads to production of spliced RNA that cannot be packaged into vector virions. Splicing from the MSD to cellular transcripts from transcription read-through products derived from integrated vectors in transduced cells has also been reported, raising safety concerns. The present inventors have previously described novel mutations within the MSD splicing region that lead to less pronounced reduction in vector titres (in the absence of modified U1 snRNA) leading to further increases in titres in the presence of modified U1 snRNAs. Such a mutation or deletion of the major splice donor site may have additional improved effects on vector titre to those described herein, and may be used in combination with any other aspect of the invention as described herein.

RNA splicing is catalysed by a large RNA-protein complex called the spliceosome, which is comprised of five small nuclear ribonucleoproteins (snRNPs). The borders between introns and exons are marked by specific nucleotide sequences within a pre-mRNA, which delineate where splicing will occur. Such boundaries are referred to as “splice sites.” The term “splice site” refers to polynucleotides that are capable of being recognized by the splicing machinery of a eukaryotic cell as suitable for being cut and/or ligated to another splice site.

Splice sites allow for the excision of introns present in a pre-mRNA transcript. Typically, the 5′ splice boundary is referred to as the “splice donor site” or the “5′ splice site,” and the 3′ splice boundary is referred to as the “splice acceptor site” or the “3′ splice site.” Splice sites include, for example, naturally occurring splice sites, engineered or synthetic splice sites, canonical or consensus splice sites, and/or non-canonical splice sites, for example, cryptic splice sites.

Splice acceptor sites generally consist of three separate sequence elements: the branch point or branch site, a polypyrimidine tract and the acceptor consensus sequence. The branch point consensus sequence in eukaryotes is YNYTRAC ((SEQ ID NO: 5) where Y is a pyrimidine, N is any nucleotide, and R is a purine). The 3′ acceptor splice site consensus sequence is YAG ((SEQ ID NO: 6) where Y is a pyrimidine) (see, e.g., Griffiths et al., eds., Modern Genetic Analysis, 2nd edition, W.H. Freeman and Company, New York (2002)). The 3′ splice acceptor site typically is located at the 3′ end of an intron.

As such, the major splice donor site may be inactivated in the nucleotide sequence encoding an RNA genome of a lentiviral vector for use in the methods, systems and uses described herein.

In other words, the cells that are used in the methods, systems and uses described herein may comprise nucleic acid sequences encoding lentiviral vector components (e.g. gag, env, rev, and/or the RNA genome of a lentiviral vector) wherein the major splice donor site in the RNA genome of the lentiviral vector is inactivated, for example is mutated or deleted.

The terms “canonical splice site” or “consensus splice site” may be used interchangeably and refer to splice sites that are conserved across species.

Consensus sequences for the 5′ donor splice site and the 3′ acceptor splice site used in eukaryotic RNA splicing are well known in the art. These consensus sequences include nearly invariant dinucleotides at each end of the intron: GT at the 5′ end of the intron, and AG at the 3′ end of an intron.

The canonical splice donor site consensus sequence may be (for DNA) AG/GTRAGT (SEQ ID NO: 7) (where A is adenosine, T is thymine, G is guanine, C is cytosine, R is a purine and “/” indicates the cleavage site). This conforms to the more general splice donor consensus sequence MAGGURR (SEQ ID NO: 1) described herein. It is well known in the art that a splice donor sequence may deviate from this consensus, especially in viral genomes where other constraints bear on the same sequence, such as secondary structure for example within a vRNA packaging region. Non-canonical splice sites are also well known in the art, albeit they occur rarely compared to the canonical splice donor consensus sequence.

The term “major splice donor site” refers to the first (dominant) splice donor site in the viral vector genome, encoded and embedded within the native viral RNA packaging sequence typically located in the 5′ region of the viral vector nucleotide sequence.

In one aspect the viral vector genome does not contain an active major splice donor site, that is splicing does not occur from the major splice donor site in said nucleotide sequence, and splicing activity from the major splice donor site is ablated.

The major splice donor site is located in the 5′ packaging region of a lentiviral genome. In the case of the HIV-1 virus, the major splice donor consensus sequence is (for DNA) TG/GTRAGT ((SEQ ID NO: 8) where A is adenosine, T is thymine, G is guanine, C is cytosine, R is a purine and “/” indicates the cleavage site).

The splice donor region, i.e. the region of the vector genome which comprises the major splice donor site prior to mutation may have the following sequence:

(SEQ ID NO: 9)
GGGGCGGCGACTGGTGAGTACGCCAAAAAT

In one example, the mutated splice donor region may comprise the sequence:

(SEQ ID NO: 10, MSD-2KO)
GGGGCGGCGACTGCAGACAACGCCAAAAAT

In one example, the mutated splice donor region may comprise the sequence:

(SEQ ID NO: 11, MSD-2KOv2)
GGGGCGGCGAGTGGAGACTACGCCAAAAAT

In another example, the mutated splice donor region may comprise the sequence:

(SEQ ID NO: 12, MSD-2KOm5)
GGGGAAGGCAACAGATAAATATGCCTTAAAAT

In one example, prior to modification the splice donor region may comprise the sequence:

(SEQ ID NO: 13)
GGCGACTGGTGAGTACGCC

This sequence is also referred to herein as the “stem loop 2” region (SL2). This sequence may form a stem loop structure in the splice donor region of the vector genome. In one example, this sequence (SL2) may have been deleted from the nucleotide sequence described herein.

As such, a nucleotide sequence that does not comprise SL2 may be used. A nucleotide sequence that does not comprise a sequence according to SL2 above may also be used.

The major splice donor site may have the following consensus sequence, wherein R is a purine and “/” is the cleavage site:

(SEQ ID NO: 8)
TG/GTRAGT

In one example, R may be guanine (G).

The major splice donor and cryptic splice donor region may have the following core sequence, wherein “/” are the cleavage sites at the major splice donor and cryptic splice donor sites:

(SEQ ID NO: 14)
/GTGA/GTA

In one example, the MSD-mutated vector genome may have at least two mutations in the major splice donor and cryptic splice donor ‘region’, wherein the first and second ‘GT’ nucleotides are immediately 3′ of the major splice donor and cryptic splice donor nucleotides respectively.

In one aspect of the invention the major splice donor consensus sequence is CTGGT (SEQ ID NO: 15). The major splice donor site may contain the sequence CTGGT (SEQ ID NO: 15).

In one aspect the nucleotide sequence comprises an inactivated major splice donor site which would otherwise have a cleavage site between nucleotides corresponding to nucleotides 13 and 14 of GGGGCGGCGACTGGTGAGTACGCCAAAAAT (SEQ ID NO: 9).

As described herein, the nucleotide sequence may also contain an inactive cryptic splice donor site. In one aspect the nucleotide sequence does not contain an active cryptic splice donor site adjacent to (3′ of) the major splice donor site, that is to say that splicing does not occur from the adjacent cryptic splice donor site, and splicing from the cryptic splice donor site is ablated.

The term “cryptic splice donor site” refers to a nucleic acid sequence which does not normally function as a splice donor site or is utilised less efficiently as a splice donor site due to the adjacent sequence context (e.g. the presence of a nearby ‘preferred’ splice donor), but can be activated to become a more efficient functioning splice donor site by mutation of the adjacent sequence (e.g. mutation of the nearby ‘preferred’ splice donor).

In one aspect the cryptic splice donor site is the first cryptic splice donor site 3′ of the major splice donor.

In one aspect the cryptic splice donor site is within 6 nucleotides of the major splice donor site on the 3′ side of the major splice donor site. Preferably the cryptic splice donor site is within 4 or 5, preferably 4, nucleotides of the major splice donor cleavage site.

In one aspect of the invention the cryptic splice donor site has the consensus sequence TGAGT (SEQ ID NO: 16).

In one aspect the nucleotide sequence comprises an inactivated cryptic splice donor site which would otherwise have a cleavage site between nucleotides corresponding to nucleotides 17 and 18 of GGGGCGGCGACTGGTGAGTACGCCAAAAAT (SEQ ID NO: 9).

In one aspect of the invention the major splice donor site and/or adjacent cryptic splice donor site contain a “GT” motif. In one aspect of the invention both the major splice donor site and adjacent cryptic splice donor site contain a “GT” motif which is mutated. The mutated GT motifs may inactivate splice activity from both the major splice donor site and adjacent cryptic splice donor site. An example of such a mutation is referred to herein as “MSD-2K0”.

In one aspect the splice donor region may comprise the following sequence:

(SEQ ID NO: 17)
CAGACA

For example, in one aspect the mutated splice donor region may comprise the following sequence:

(SEQ ID NO: 18)
GGCGACTGCAGACAACGCC

A further example of an inactivating mutation is referred to herein as “MSD-2KOv2”.

In one aspect the mutated splice donor region may comprise the following sequence:

(SEQ ID NO: 19)
GTGGAGACT

For example, in one aspect the mutated splice donor region may comprise the following sequence:

(SEQ ID NO: 20)
GGCGAGTGGAGACTACGCC

For example, in one aspect the mutated splice donor region may comprise the following sequence:

(SEQ ID NO: 21)
AAGGCAACAGATAAATATGCCTT

In one aspect the stem loop 2 region as described above may be deleted from the splice donor region, resulting in inactivation of both the major splice donor site and the adjacent cryptic splice donor site. Such a deletion is referred to herein as “ΔSL2”.

A variety of different types of mutations can be introduced into the viral vector nucleotide sequence in order to inactivate the major and adjacent cryptic splice donor sites.

In one aspect the mutation is a functional mutation to ablate or suppress splicing activity in the splice region. Suitable mutations will be known to one skilled in the art, and are described herein.

For example, a point mutation can be introduced into the nucleic acid sequence. The term “point mutation,” as used herein, refers to any change to a single nucleotide. Point mutations include, for example, deletions, transitions, and transversions; these can be classified as nonsense mutations, missense mutations, or silent mutations when present within protein coding sequence. A “nonsense” mutation produces a stop codon. A “missense” mutation produces a codon that encodes a different amino acid. A “silent” mutation produces a codon that encodes either the same amino acid or a different amino acid that does not alter the function of the protein. One or more point mutations can be introduced into the nucleic acid sequence comprising the cryptic splice donor site. For example, the nucleic acid sequence comprising the cryptic splice site can be mutated by introducing two or more point mutations therein.

At least two point mutations can be introduced in several locations within the nucleic acid sequence comprising the major splice donor and cryptic splice donor sites to achieve attenuation of splicing from the splice donor region. In one aspect the mutations may be within the four nucleotides at the splice donor cleavage site; in the canonical splice donor consensus sequence this is A1G2/G3T4, wherein “/” is the cleavage site. It is well known in the art that a splice donor cleavage site may deviate from this consensus, especially in viral genomes where other constraints bear on the same sequence, such as secondary structure for example within a vRNA packaging region. It is well known that the G3T4 dinucleotide is generally the least variable sequence within the canonical splice donor consensus sequence, and mutations to the G3 and or T4 will most likely achieve the greatest attenuating effect. For example, for the major splice donor site in HIV-1 viral vector genomes this can be T1G2/G3T4, wherein “/” is the cleavage site. For example, for the cryptic splice donor site in HIV-1 viral vector genomes this can be G1A2/G3T4, wherein “/” is the cleavage site. Additionally, the point mutation(s) can be introduced adjacent to a splice donor site. For example, the point mutation can be introduced upstream or downstream of a splice donor site. In embodiments where the nucleic acid sequence comprising a major and/or cryptic splice donor site is mutated by introducing multiple point mutations therein, the point mutations can be introduced upstream and/or downstream of the cryptic splice donor site.

A nucleotide sequence encoding the RNA genome of a lentiviral vector may therefore be used in the methods, systems and uses described herein, wherein the major splice donor site in the RNA genome of the lentiviral vector is inactivated, and wherein the cryptic splice donor site 3′ to the major splice donor site is inactivated.

Suitably, the lentiviral vector may be a third generation lentiviral vector.

Suitably, the cryptic splice donor site may be the first cryptic splice donor site 3′ to the major splice donor site.

Suitably, said cryptic splice donor site may be within 6 nucleotides of the major splice donor site.

Suitably, the major splice donor site and cryptic splice donor site may be mutated or deleted and/or the splicing activity from the major splice donor site and cryptic splice donor site of the RNA genome of the lentiviral vector may be suppressed or ablated (e.g. in transfected cells or in transduced cells).

Construction of Splice Site Mutants

Splice site mutants of the present invention may be constructed using a variety of techniques. For example, mutations may be introduced at particular loci by synthesising oligonucleotides containing a mutant sequence, flanked by restriction sites enabling ligation to fragments of the native sequence. Following ligation, the resulting reconstructed sequence comprises a derivative having the desired nucleotide insertion, substitution, or deletion.

Other known techniques allowing alterations of DNA sequence include recombination approaches such as Gibson assembly, Golden-gate cloning and In-fusion.

Alternatively, oligonucleotide-directed site-specific (or segment specific) mutagenesis procedures may be employed to provide an altered sequence according to the substitution, deletion, or insertion required. Deletion or truncation derivatives of splice site mutants may also be constructed by utilising convenient restriction endonuclease sites adjacent to the desired deletion.

Subsequent to restriction, overhangs may be filled in, and the DNA re-ligated.

Exemplary methods of making the alterations set forth above are disclosed by Sambrook et al. (Molecular cloning: A Laboratory Manual, 2d Ed., Cold Spring Harbor Laboratory Press, 1989). Splice site mutants may also be constructed utilising techniques of PCR mutagenesis, chemical mutagenesis, chemical mutagenesis (Drinkwater and Klinedinst, 1986) by forced nucleotide misincorporation (e.g., Liao and Wise, 1990), or by use of randomly mutagenised oligonucleotides (Horwitz et al., 1989).

D. Tat Independent Lentiviral Vectors

In the context of lentiviral vector production specifically, a tat-independent lentiviral vector may be used with the methods, viral vector production systems, and uses comprising PKC activators (and optionally HDAC inhibitors) described herein. In one aspect the lentiviral vector may be a 3^{rd} generation lentiviral vector. For clarity it is understood that the term ‘tat-independent’ means that the HIV-1 U3 promoter used to drive transcription of the vector genome cassette is replaced by a heterologous promoter. In one aspect, tat is not provided in the lentiviral vector production method, system or use, for example tat is not provided in trans. In one aspect the cell or vector or vector production system as described herein does not comprise the tat protein.

### Definitions

The practice of the present invention will employ, unless otherwise indicated, conventional techniques of chemistry, molecular biology, microbiology and immunology, which are within the capabilities of a person of ordinary skill in the art. Such techniques are explained in the literature. See, for example, J. Sambrook, E. F. Fritsch, and T. Maniatis (1989) Molecular Cloning: A Laboratory Manual, Second Edition, Books 1-3, Cold Spring Harbor Laboratory Press; Ausubel, F. M. et al. (1995 and periodic supplements) Current Protocols in Molecular Biology, Ch. 9, 13, and 16, John Wiley & Sons, New York, N.Y.; B. Roe, J. Crabtree, and A. Kahn (1996) DNA Isolation and Sequencing: Essential Techniques, John Wiley & Sons; J. M. Polak and James O′D. McGee (1990) In Situ Hybridization: Principles and Practice; Oxford University Press; M. J. Gait (ed.) (1984) Oligonucleotide Synthesis: A Practical Approach, IRL Press; and, D. M. J. Lilley and J. E. Dahlberg (1992) Methods of Enzymology: DNA Structure Part A: Synthesis and Physical Analysis of DNA Methods in Enzymology, Academic Press. Each of these general texts is herein incorporated by reference.

Unless defined otherwise, all technical and scientific terms used herein have the same meaning as is commonly understood by one of skill in the art to which this invention belongs.

The term “protein”, as used herein, includes proteins, polypeptides, and peptides. As used herein, the term “protein” includes single-chain polypeptide molecules as well as multiple-polypeptide complexes where individual constituent polypeptides are linked by covalent or non-covalent means. As used herein, the terms “polypeptide” and “peptide” refer to a polymer in which the monomers are amino acids and are joined together through peptide or disulfide bonds.

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”.

The methods, systems and uses described herein may comprise one of the specified PKC activators, or an analogue, derivative or pharmaceutical salt thereof. The methods, systems and uses described herein may also comprise one of the specified HDAC inhibitors, or an analogue, derivative or pharmaceutical salt thereof.

The term “analogue” encompasses structural analogues. As used herein, the term “structural analogue” means a compound that shares structural characteristics with a specified compound, but differs structurally in other ways, such as the inclusion or deletion of one or more other chemical moieties.

The term “derivative” can mean a molecule that has been altered in a way which does not affects its biological activity. A derivative may be a functional derivative or a biologically effective analogue of the parent molecule.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Vector/Expression Cassette

A vector is a tool that allows or facilitates the transfer of an entity from one environment to another. In accordance with the present invention, and by way of example, some vectors used in recombinant nucleic acid techniques allow entities, such as a segment of nucleic acid (e.g. a heterologous DNA segment, such as a heterologous cDNA segment), to be transferred into and expressed by a target cell. Vectors may facilitate integration of a nucleotide sequence within the target cell. For example, the vector may facilitate the integration of the nucleotide sequence encoding the modified U1 snRNA described herein to maintain the nucleotide sequence encoding the modified U1 snRNA of the invention and its expression within the target cell.

The vector may contain one or more selectable marker genes (e.g. a neomycin resistance gene) and/or traceable marker gene(s) (e.g. a gene encoding green fluorescent protein (GFP)). Vectors may be used, for example, to infect and/or transduce a target cell. The vector may further comprise a nucleotide sequence enabling the vector to replicate in the host cell in question.

The vector may be or may include an expression cassette (also termed an expression construct). Expression cassettes as described herein comprise regions of nucleic acid containing sequences capable of being transcribed. Thus, sequences encoding mRNA, tRNA and rRNA are included within this definition.

The term “cassette”—which is synonymous with terms such as “conjugate”, “construct” and “hybrid”—includes a polynucleotide sequence directly or indirectly attached to a promoter.

Expression cassettes typically comprise a promoter for the expression of the encoded nucleotide sequence and optionally a regulator of the encoded nucleotide sequence. For example, expression cassettes encoding a viral vector component typically comprise a promoter for the expression of the nucleotide sequence encoding a viral vector component and optionally a regulator of the nucleotide sequence encoding the viral vector component. Preferably the cassette comprises at least a polynucleotide sequence operably linked to a promoter.

In the context of methods, systems or uses that comprise a modified U1 snRNA described herein, an expression cassette may be used to provide the modified U1 snRNA to the host cell. For example, an expression cassette may comprise a promoter for the expression of the nucleotide sequence encoding the modified U1 snRNA and optionally a regulator of the nucleotide sequence encoding the modified U1 snRNA. The expression cassette may be used to replicate the nucleotide sequence encoding the modified U1 snRNA in a compatible target cell in vitro. Thus, modified U1 snRNAs may be made in vitro by introducing an expression cassette encoding the modified U1 snRNA into a compatible target cell in vitro and growing the target cell under conditions which result in expression of the modified U1 snRNAs. The introduction of an expression cassette of the invention into a cell using conventional molecular and cell biology techniques is within the capabilities of a person of ordinary skill in the art. The modified U1 snRNAs may be recovered from the target cell by methods well known in the art. Suitable target cells include mammalian cell lines and other eukaryotic cell lines.

The choice of expression cassette, e.g. plasmid, cosmid, virus or phage vector, will often depend on the host cell into which it is to be introduced. The expression cassette can be a DNA plasmid (supercoiled, nicked or linearised), minicircle DNA (linear or supercoiled), plasmid DNA containing just the regions of interest by removal of the plasmid backbone by restriction enzyme digestion and purification, DNA generated using an enzymatic DNA amplification platform e.g. doggybone DNA (dbDNA™) where the final DNA used is in a closed ligated form or where it has been prepared (e.g. restriction enzyme digestion) to have open cut ends.

The methods, viral vector production systems, and uses described herein are for the production of a viral vector. As would be clear to a person of skill in the art, any appropriate viral vector may be produced by the methods, viral vector production systems and uses described herein. For example, appropriate viral vectors may be selected from the group consisting of: a retroviral vector, an adenoviral vector, an adeno-associated viral vector, a herpes simplex viral vector and a vaccinia viral vector. In one example, the viral vector is a self-inactivating (SIN) viral vector.

Adenoviral and Adeno-Associated Viral Vectors

Adenoviruses may also be detected using the methods described herein. An adenovirus is a double-stranded, linear DNA virus that does not replicate through an RNA intermediate. There are over 50 different human serotypes of adenovirus divided into 6 subgroups based on their genetic sequence.

Adenoviruses are double-stranded DNA non-enveloped viruses that are capable of in vivo, ex vivo and in vitro transduction of a broad range of cell types of human and non-human origin. These cells include respiratory airway epithelial cells, hepatocytes, muscle cells, cardiac myocytes, synoviocytes, primary mammary epithelial cells and post-mitotically terminally differentiated cells such as neurons.

Adenoviral vectors are also capable of transducing non-dividing cells. This is very important for diseases, such as cystic fibrosis, in which the affected cells in the lung epithelium have a slow turnover rate. In fact, several trials are underway utilising adenovirus-mediated transfer of cystic fibrosis transporter (CFTR) into the lungs of afflicted adult cystic fibrosis patients.

Adenoviruses have been used as vectors for gene therapy and for expression of heterologous genes. The large (36 kb) genome can accommodate up to 8 kb of foreign insert DNA and is able to replicate efficiently in complementing cell lines to produce very high titres of up to 1012 transducing units per ml. Adenovirus is thus one of the best systems to study the expression of genes in primary non-replicative cells.

The expression of viral or foreign genes from the adenovirus genome does not require a replicating cell. Adenoviral vectors enter cells by receptor mediated endocytosis. Once inside the cell, adenovirus vectors rarely integrate into the host chromosome. Instead, they function episomally (independently from the host genome) as a linear genome in the host nucleus.

The use of recombinant adeno-associated viral (AAV) and Adenovirus based viral vectors for gene therapy is widespread, and manufacture of the same has been well documented. Typically, AAV-based vectors are produced in mammalian cell lines (e.g. HEK293-based) or through use of the baculovirus/Sf9 insect cell system. AAV vectors can be produced by transient transfection of vector component encoding DNAs, typically together with helper functions from Adenovirus or Herpes Simplex virus (HSV), or by use of cell lines stably expressing AAV vector components. Adenoviral vectors are typically produced in mammalian cell lines that stably express Adenovirus E1 functions (e.g. HEK293-based).

Adenoviral vectors are also typically ‘amplified’ via helper-function-dependent replication through serial rounds of ‘infection’ using the production cell line. An adenoviral vector and production system thereof comprises a polynucleotide comprising all or a portion of an adenovirus genome. It is well known that an adenovirus is, without limitation, an adenovirus derived from Ad2, Ad5, Ad12, and Ad40. An adenoviral vector is typically in the form of DNA encapsulated in an adenovirus coat or adenoviral DNA packaged in another viral or viral-like form (such as herpes simplex, and AAV).

An AAV vector it is commonly understood to be a vector derived from an adeno-associated virus serotype, including without limitation, AAV-1, AAV-2, AAV-3, AAV-4, AAV-5, AAV-6, AAV-7 and AAV-8. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging. An ‘AAV vector’ also refers to its protein shell or capsid, which provides an efficient vehicle for delivery of vector nucleic acid to the nucleus of target cells. AAV production systems require helper functions which typically refers to AAV-derived coding sequences which can be expressed to provide AAV gene products that, in turn, function in trans for productive AAV replication. As such, AAV helper functions include both of the major AAV open reading frames (ORFs), rep and cap. The Rep expression products have been shown to possess many functions, including, among others: recognition, binding and nicking of the AAV origin of DNA replication; DNA helicase activity; and modulation of transcription from AAV (or other heterologous) promoters. The Cap expression products supply necessary packaging functions. AAV helper functions are used herein to complement AAV functions in trans that are missing from AAV vectors. It is understood that a AAV helper construct refers generally to a nucleic acid molecule that includes nucleotide sequences providing AAV functions deleted from an AAV vector which is to be used to produce a transducing vector for delivery of a nucleotide sequence of interest. AAV helper constructs are commonly used to provide transient expression of AAV rep and/or cap genes to complement missing AAV functions that are necessary for AAV replication; however, helper constructs lack AAV ITRs and can neither replicate nor package themselves. AAV helper constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. See, e.g., Samulski et al. (1989) J. Virol. 63:3822-3828; and McCarty et al. (1991) J. Virol. 65:2936-2945. A number of other vectors have been described which encode Rep and/or Cap expression products. See, e.g., U.S. Pat. Nos. 5,139,941 and 6,376,237. In addition, it is common knowledge that the term “accessory functions” refers to non-AAV derived viral and/or cellular functions upon which AAV is dependent for its replication. Thus, the term captures proteins and RNAs that are required in AAV replication, including those moieties involved in activation of AAV gene transcription, stage specific AAV mRNA splicing, AAV DNA replication, synthesis of Cap expression products and AAV capsid assembly. Viral-based accessory functions can be derived from any of the known helper viruses such as adenovirus, herpesvirus (other than herpes simplex virus type-1) and vaccinia virus.

Herpes Simplex Virus Vectors

Herpes simplex virus (HSV) is an enveloped double-stranded DNA virus that naturally infects neurons. It can accommodate large sections of foreign DNA, which makes it attractive as a vector system, and has been employed as a vector for gene delivery to neurons (Manservigiet et al Open Virol J. (2010) 4:123-156).

The use of HSV in therapeutic procedures requires the strains to be attenuated so that they cannot establish a lytic cycle. In particular, if HSV vectors are used for gene therapy in humans, the polynucleotide should preferably be inserted into an essential gene. This is because if a viral vector encounters a wild-type virus, transfer of a heterologous gene to the wild-type virus could occur by recombination. However, as long as the polynucleotide is inserted into an essential gene, this recombinational transfer would also delete the essential gene in the recipient virus and prevent “escape” of the heterologous gene into the replication competent wild-type virus population.

Vaccinia Virus Vectors

Methods described herein may also be used to detect the presence of a replication competent vaccinia virus. Vaccinia virus vectors include MVA or NYVAC. Alternatives to vaccinia vectors include avipox vectors such as fowlpox or canarypox known as ALVAC and strains derived therefrom which can infect and express recombinant proteins in human cells but are unable to replicate.

In another example, the viral vector is a retroviral vector, preferably the retroviral vector is a lentiviral vector (e.g. a SIN lentiviral vector). Further details of these viruses are provided elsewhere herein. Suitable lentiviral vectors may be selected from the group consisting of: HIV-1, HIV-2, SIV, FIV, BIV, EIAV, CAEV and visna lentiviral vector. For example, the lentiviral vector may be selected from an HIV (e.g. HIV-1, HIV-2) or an EIAV lentiviral vector.

Retroviral Vectors

Retroviral vectors may be derived from or may be derivable from any suitable retrovirus. A large number of different retroviruses have been identified. Examples include: murine leukemia virus (MLV), human T-cell leukemia virus (HTLV), mouse mammary tumour virus (MMTV), Rous sarcoma virus (RSV), Fujinami sarcoma virus (FuSV), Moloney murine leukemia virus (Mo MLV), FBR murine osteosarcoma virus (FBR MSV), Moloney murine sarcoma virus (Mo-MSV), Abelson murine leukemia virus (A-MLV), Avian myelocytomatosis virus-29 (MC29) and Avian erythroblastosis virus (AEV). A detailed list of retroviruses may be found in Coffin et al. (1997) “Retroviruses”, Cold Spring Harbour Laboratory Press Eds: J M Coffin, SM Hughes, HE Varmus pp 758-763.

Retroviruses may be broadly divided into two categories, namely “simple” and “complex”. Retroviruses may even be further divided into seven groups. Five of these groups represent retroviruses with oncogenic potential. The remaining two groups are the lentiviruses and the spumaviruses. A review of these retroviruses is presented in Coffin et al (1997) ibid.

The basic structure of retroviral and lentiviral genomes share many common features such as a 5′ LTR and a 3′ LTR, between or within which are located a packaging signal to enable the genome to be packaged, a primer binding site, integration sites to enable integration into a target cell genome and gag/pol and env genes encoding the packaging components—these are polypeptides required for the assembly of viral particles. Lentiviruses have additional features, such as the rev gene and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration, and transcription. LTRs also serve as enhancer-promoter sequences and can control the expression of the viral genes.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

In a typical retroviral vector, at least part of one or more protein coding regions essential for replication may be removed from the virus; for example, gag/pol and env may be absent or not functional. This makes the viral vector replication-defective.

Lentiviral Vectors

Lentiviruses are part of a larger group of retroviruses. A detailed list of lentiviruses may be found in Coffin et al (1997) “Retroviruses” Cold Spring Harbour Laboratory Press Eds: J M Coffin, SM Hughes, HE Varmus pp 758-763). In brief, lentiviruses can be divided into primate and non-primate groups. Examples of primate lentiviruses include but are not limited to: the human immunodeficiency virus (HIV), the causative agent of human auto-immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV), feline immunodeficiency virus (FIV), Maedi visna virus (MVV) and bovine immunodeficiency virus (BIV).

The lentivirus family differs from retroviruses in that lentiviruses have the capability to infect both dividing and non-dividing cells (Lewis et al (1992) EMBO J 11(8):3053-3058 and Lewis and Emerman (1994) J Virol 68 (1):510-516). In contrast, other retroviruses, such as MLV, are unable to infect non-dividing or slowly dividing cells such as those that make up, for example, muscle, brain, lung and liver tissue.

A lentiviral vector, as used herein, is a vector which comprises at least one component part derivable from a lentivirus. Preferably, that component part is involved in the biological mechanisms by which the vector infects or transduces target cells and expresses NOI.

The lentiviral vector may be used to replicate the NOI in a compatible target cell in vitro. Thus, described herein is a method of making proteins in vitro by introducing a vector of the invention into a compatible target cell in vitro and growing the target cell under conditions which result in expression of the NOI. Protein and NOI may be recovered from the target cell by methods well known in the art. Suitable target cells include mammalian cell lines and other eukaryotic cell lines and are described elsewhere herein.

The vectors may have “insulators”—genetic sequences that block the interaction between promoters and enhancers, and act as a barrier reducing read-through from an adjacent gene. The insulator may be present between one or more of the lentiviral nucleic acid sequences to prevent promoter interference and read-thorough from adjacent genes. If the insulators are present in the vector between one or more of the lentiviral nucleic acid sequences, then each of these insulated genes may be arranged as individual expression units.

The basic structure of retroviral and lentiviral genomes share many common features such as a 5′ LTR and a 3′ LTR, between or within which are located a packaging signal to enable the genome to be packaged, a primer binding site, integration sites to enable integration into a target cell genome and gag/pol and env genes encoding the packaging components—these are polypeptides required for the assembly of viral particles. Lentiviruses have additional features, such as the rev gene and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration, and transcription. LTRs also serve as enhancer-promoter sequences and can control the expression of the viral genes.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

In a typical lentiviral vector as described herein, at least part of one or more protein coding regions essential for replication may be removed from the virus; for example, gag/pol and env may be absent or not functional. This makes the viral vector replication-defective.

The lentiviral vector may be derived from either a primate lentivirus (e.g. HIV-1) or a non-primate lentivirus (e.g. EIAV).

In general terms, a typical retroviral vector production system involves the separation of the viral genome from the essential viral packaging functions. These components are normally provided to the production cells on separate DNA expression cassettes (alternatively known as plasmids, expression plasmids, DNA constructs or expression constructs).

The vector genome comprises the NOI. Vector genomes typically require a packaging signal (ψ), the internal expression cassette harbouring the NOI, (optionally) a post-transcriptional element (PRE), typically a central polypurine tract (cppt), the 3′-ppu and a self-inactivating (SIN) LTR. The R-U5 regions are required for correct polyadenylation of both the vector genome RNA and NOI mRNA, as well as the process of reverse transcription. The vector genome may optionally include an open reading frame, as described in WO 2003/064665, which allows for vector production in the absence of rev.

The packaging functions include the gag/pol and env genes. These are required for the production of vector particles by the production cell. Providing these functions in trans to the genome facilitates the production of replication-defective viral vectors.

Production systems for gamma-retroviral vectors are typically 3-component systems requiring genome, gag/pol and env expression constructs. Production systems for HIV-1-based lentiviral vectors may additionally require the accessory gene rev to be provided and for the vector genome to include the rev-responsive element (RRE). EIAV-based lentiviral vectors do not require rev to be provided in trans if an open-reading frame (ORF) is present within the genome (see WO 2003/064665).

Usually both the “external” promoter (which drives the vector genome cassette) and “internal” promoter (which drives the NOI cassette) encoded within the vector genome cassette are strong eukaryotic or virus promoters, as are those driving the other vector system components. Examples of such promoters include CMV, EF1a, PGK, CAG, TK, SV40 and Ubiquitin promoters. Strong ‘synthetic’ promoters, such as those generated by DNA libraries (e.g. JeT promoter) may also be used to drive transcription. Alternatively, tissue-specific promoters such as rhodopsin (Rho), rhodopsin kinase (RhoK), cone-rod homeobox containing gene (CRX), neural retina-specific leucine zipper protein (NRL), Vitelliform Macular Dystrophy 2 (VMD2), Tyrosine hydroxylase, neuronal-specific neuronal-specific enolase (NSE) promoter, astrocyte-specific glial fibrillary acidic protein (GFAP) promoter, human al-antitrypsin (hAAT) promoter, phosphoenolpyruvate carboxykinase (PEPCK), liver fatty acid binding protein promoter, Flt-1 promoter, INF-β promoter, Mb promoter, SP-B promoter, SYN1 promoter, WASP promoter, SV40/hAIb promoter, SV40/CD43, SV40/CD45, NSE/RU5′ promoter, ICAM-2 promoter, GPIIb promoter, GFAP promoter, Fibronectin promoter, Endoglin promoter, Elastase-1 promoter, Desmin promoter, CD68 promoter, CD14 promoter and B29 promoter may be used to drive transcription.

Production of viral vectors involves either the transient co-transfection of the production cells with these DNA components or use of stable production cell lines wherein all the components are stably integrated within the production cell genome (e.g. Stewart H J, Fong-Wong L, Strickland I, Chipchase D, Kelleher M, Stevenson L, Thoree V, McCarthy J, Ralph G S, Mitrophanous K A and Radcliffe P A. (2011). Hum Gene Ther. March; 22 (3):357-69). An alternative approach is to use a stable packaging cell (into which the packaging components are stably integrated) and then transiently transfect in the vector genome plasmid as required (e.g. Stewart, H. J., M. A. Leroux-Carlucci, C. J. Sion, K. A. Mitrophanous and P. A. Radcliffe (2009). Gene Ther. June; 16 (6):805-14). It is also feasible that alternative, not complete, packaging cell lines could be generated (just one or two packaging components are stably integrated into the cell lines) and to generate vector the missing components are transiently transfected. The production cell may also express regulatory proteins such as a member of the tet repressor (TetR) protein group of transcription regulators (e.g. T-Rex, Tet-On, and Tet-Off), a member of the cumate inducible switch system group of transcription regulators (e.g. cumate repressor (CymR) protein), or an RNA-binding protein (e.g. TRAP—tryptophan-activated RNA-binding protein).

In one example, the viral vector is derived from EIAV. EIAV has the simplest genomic structure of the lentiviruses and is particularly preferred for use in the present invention. In addition to the gag/pol and env genes, EIAV encodes three other genes: tat, rev, and S2. Tat acts as a transcriptional activator of the viral LTR (Derse and Newbold (1993) Virology 194(2):530-536 and Maury et al (1994) Virology 200(2):632-642) and rev regulates and coordinates the expression of viral genes through rev-response elements (RRE) (Martarano et al. (1994) J Virol 68(5):3102-3111). The mechanisms of action of these two proteins are thought to be broadly similar to the analogous mechanisms in the primate viruses (Martarano et al. (1994) J Virol 68(5):3102-3111). The function of S2 is unknown. In addition, an EIAV protein, Ttm, has been identified that is encoded by the first exon of tat spliced to the env coding sequence at the start of the transmembrane protein. In an alternative embodiment of the present invention the viral vector is derived from HIV: HIV differs from EIAV in that it does not encode S2 but unlike EIAV it encodes vif, vpr, vpu and nef.

The term “recombinant retroviral or lentiviral vector” (RRV) refers to a vector with sufficient retroviral genetic information to allow packaging of an RNA genome, in the presence of packaging components, into a viral particle capable of transducing a target cell.

Transduction of the target cell may include reverse transcription and integration into the target cell genome. The RRV carries non-viral coding sequences which are to be delivered by the vector to the target cell. A RRV is incapable of independent replication to produce infectious retroviral particles within the target cell. Usually the RRV lacks a functional gag/pol and/or env gene, and/or other genes essential for replication.

Preferably the RRV vector of the present invention has a minimal viral genome.

As used herein, the term “minimal viral genome” means that the viral vector has been manipulated so as to remove the non-essential elements whilst retaining the elements essential to provide the required functionality to infect, transduce and deliver a NOI to a target cell. Further details of this strategy can be found in WO 1998/17815 and WO 99/32646. A minimal EIAV vector lacks tat, rev and S2 genes and neither are these genes provided in trans in the production system. A minimal HIV vector lacks vif, vpr, vpu, tat and nef.

The expression cassette used to produce the vector genome within a production cell may include transcriptional regulatory control sequences operably linked to the retroviral genome to direct transcription of the genome in a production cell/packaging cell. The expression plasmid used to produce the vector genome within a production cell may include transcriptional regulatory control sequences operably linked to the retroviral genome to direct transcription of the genome in a production cell/packaging cell. All 3^{rd} generation lentiviral vectors are deleted in the 5′ U3 enhancer-promoter region, and transcription of the vector genome RNA is driven by heterologous promoter such as another viral promoter, for example the CMV promoter, as discussed below. This feature enables vector production independently of tat. Some lentiviral vector genomes require additional sequences for efficient virus production. For example, particularly in the case of HIV, RRE sequences may be included. However the requirement for RRE on the (separate) GagPol cassette (and dependence on rev which is provided in trans) may be reduced or eliminated by codon optimisation of the GagPol ORF. Further details of this strategy can be found in WO 2001/79518.

Alternative sequences which perform the same function as the rev/RRE system are also known. For example, a functional analogue of the rev/RRE system is found in the Mason Pfizer monkey virus. This is known as the constitutive transport element (CTE) and comprises an RRE-type sequence in the genome which is believed to interact with a factor in the infected cell. The cellular factor can be thought of as a rev analogue. Thus, CTE may be used as an alternative to the rev/RRE system. Any other functional equivalents of the Rev protein which are known or become available may be relevant to the invention. For example, it is also known that the Rex protein of HTLV-I can functionally replace the Rev protein of HIV-1. Rev and RRE may be absent or non-functional in the vector for use in the methods of the present invention; in the alternative rev and RRE, or functionally equivalent system, may be present.

As used herein, the term “functional substitute” means a protein or sequence having an alternative sequence which performs the same function as another protein or sequence. The term “functional substitute” is used interchangeably with “functional equivalent” and “functional analogue” herein with the same meaning.

SIN Vectors

The viral vectors described herein may be used in a self-inactivating (SIN) configuration in which the viral enhancer and promoter sequences have been deleted. For example, lentiviral vectors described herein may be used in a SIN configuration. SIN vectors can be generated and transduce non-dividing target cells in vivo, ex vivo or in vitro with an efficacy similar to that of non-SIN vectors. The transcriptional inactivation of the long terminal repeat (LTR) in the SIN provirus should prevent mobilisation of vRNA, and is a feature that further diminishes the likelihood of formation of replication-competent virus. This should also enable the regulated expression of genes from internal promoters by eliminating any cis-acting effects of the LTR.

By way of example, self-inactivating retroviral vector systems have been constructed by deleting the transcriptional enhancers or the enhancers and promoter in the U3 region of the 3′ LTR. After a round of vector reverse transcription and integration, these changes are copied into both the 5′ and the 3′ LTRs producing a transcriptionally inactive ‘provirus’. However, any promoter(s) internal to the LTRs in such vectors will still be transcriptionally active. This strategy has been employed to eliminate effects of the enhancers and promoters in the viral LTRs on transcription from internally placed genes. Such effects include increased transcription or suppression of transcription. This strategy can also be used to eliminate downstream transcription from the 3′ LTR into genomic DNA. This is of particular concern in human gene therapy where it is important to prevent the adventitious activation of any endogenous oncogene. Yu et al., (1986) PNAS 83: 3194-98; Marty et al., (1990) Biochimie 72: 885-7; Naviaux et al., (1996) J. Virol. 70: 5701-5; Iwakuma et al., (1999) Virol. 261: 120-32; Deglon et al., (2000) Human Gene Therapy 11: 179-90. SIN lentiviral vectors are described in U.S. Pat. Nos. 6,924,123 and 7,056,699.

Replication-Defective Vectors

In the genome of a replication-defective viral vector the sequences of gag/pol and/or env may be mutated and/or not functional.

In a typical viral vector as described herein, at least part of one or more coding regions for proteins essential for virus replication may be removed from the vector. This makes the viral vector replication-defective. Portions of the viral genome may also be replaced by a NOI in order to generate a vector comprising an NOI which is capable of transducing a non-dividing target cell and/or integrating its genome into the target cell genome.

In one example, the viral vectors are non-integrating vectors as described in WO 2006/010834 and WO 2007/071994.

In a further example, the vectors have the ability to deliver a sequence which is devoid of or lacking viral RNA. In a further example, and a cognate binding domain on Gag or GagPol can be used to ensure packaging of the RNA to be delivered. Both of these vectors are described in WO 2007/072056.

Vector Production Systems and Cells

The viral vector production systems described herein comprise a set of nucleotide sequences encoding the components required for production of the viral vector. Accordingly, a vector production system comprises a set of nucleotide sequences which encode the components necessary to generate viral vector particles. Typically, the set of nucleotide sequences is present within a cell.

“Viral vector production system” or “vector production system” or “production system” is to be understood as a system comprising the necessary components for viral vector production. The terms “components required for production of the vector” and “viral vector components” are used interchangeably herein. The viral vector production system comprises a set of nucleotide sequences which encode the components necessary to generate viral vector particles.

A non-limiting example of a viral vector production system described herein is a lentiviral vector production system. A lentiviral vector production system of the invention comprises a set of nucleotide sequences encoding the components required for production of a lentiviral vector. A lentiviral vector production system therefore comprises a set of nucleotide sequences which encode the components necessary to generate lentiviral vector particles. As stated above, the set of nucleotide sequences is typically present within a cell.

In one example, the set of nucleotide sequences may be suitable for generation of a lentiviral vector in a tat-independent system for vector production. As described herein, 3rd generation lentiviral vectors are U3-dependent (and employ a heterologous promoter to drive transcription). In one example, tat is not provided in the lentiviral vector production system, for example tat is not provided in trans. In one aspect the viral vector production system as described herein therefore does not comprise the tat protein.

In one example, the set of nucleotide sequences may comprise nucleotide sequences encoding Gag and Gag/Pol proteins, and Env protein and the vector genome sequence. The set of nucleotide sequences may optionally comprise a nucleotide sequence encoding the Rev protein, or functional substitute thereof.

In one embodiment, the viral vector production system comprises modular nucleic acid constructs (modular constructs). A modular construct is a DNA expression construct comprising two or more nucleic acids used in the production of viral vectors. A modular construct can be a DNA plasmid comprising two or more nucleic acids used in the production of viral vectors. The plasmid may be a bacterial plasmid. The nucleic acids can encode for example, gag-pol, rev, env, vector genome. In addition, modular constructs designed for generation of packaging and producer cell lines may additionally need to encode transcriptional regulatory proteins (e.g. TetR, CymR) and/or translational repression proteins (e.g. TRAP) and selectable markers (e.g. Zeocin™, hygromycin, blasticidin, puromycin, neomycin resistance genes). Suitable modular constructs are described in EP 3502260, which is hereby incorporated by reference in its entirety.

As modular constructs contain nucleic acid sequences encoding two or more of the viral components on one construct, the safety profile of these modular constructs has been considered and additional safety features directly engineered into the constructs. These features include the use of insulators for multiple open reading frames of viral vector components and/or the specific orientation and arrangement of the viral genes in the modular constructs. It is believed that by using these features the direct read-through to generate replication-competent viral particles will be prevented.

The nucleic acid sequences encoding the viral vector components may be in reverse and/or alternating transcriptional orientations in the modular construct. Thus, the nucleic acid sequences encoding the viral vector components are not presented in the same 5′ to 3′ orientation, such that the viral vector components cannot be produced from the same mRNA molecule. The reverse orientation may mean that at least two coding sequences for different vector components are presented in the ‘head-to-head’ and ‘tail-to-tail’ transcriptional orientations. This may be achieved by providing the coding sequence for one vector component, e.g. env, on one strand and the coding sequence for another vector component, e.g. rev, on the opposing strand of the modular construct. Preferably, when coding sequences for more than two vector components are present in the modular construct, at least two of the coding sequences are present in the reverse transcriptional orientation. Accordingly, when coding sequences for more than two vector components are present in the modular construct, each component may be orientated such that it is present in the opposite 5′ to 3′ orientation to all of the adjacent coding sequence(s) for other vector components to which it is adjacent, i.e. alternating 5′ to 3′ (or transcriptional) orientations for each coding sequence may be employed.

The modular construct may comprise nucleic acid sequences encoding two or more of the following vector components: gag-pol, rev, env, vector genome. The modular construct may comprise nucleic acid sequences encoding any combination of the vector components. In one example, the modular construct may comprise nucleic acid sequences encoding:

i) the RNA genome of a retroviral vector and rev, or a functional substitute thereof;

ii) the RNA genome of a retroviral vector and gag-pol;

iii) the RNA genome of a retroviral vector and env;

iv) gag-pol and rev, or a functional substitute thereof;

v) gag-pol and env;

vi) env and rev, or a functional substitute thereof;

vii) the RNA genome of a retroviral vector, rev, or a functional substitute thereof, and gag-pol;

viii) the RNA genome of a retroviral vector, rev, or a functional substitute thereof, and env;

ix) the RNA genome of a retroviral vector, gag-pol and env; or

x) gag-pol, rev, or a functional substitute thereof, and env,

wherein the nucleic acid sequences are in reverse and/or alternating orientations.

In some examples, the retroviral vector may be a lentiviral vector.

As stated elsewhere herein, the viral vector production system described herein typically comprises the nucleic acid sequences encoding viral vector components within a cell (in other words, a cell comprises the nucleic acid sequences encoding viral vector components). In one example, the cell of the viral vector production system may comprise nucleic acid sequences encoding any one of the combinations i) to x) above, wherein the nucleic acid sequences are located at the same genetic locus and are in reverse and/or alternating orientations. The same genetic locus may refer to a single extrachromosomal locus in the cell, e.g. a single plasmid, or a single locus (i.e. a single insertion site) in the genome of the cell. The cell may be a stable or transient cell for producing retroviral vectors, e.g. lentiviral vectors.

The DNA expression construct can be a DNA plasmid (supercoiled, nicked or linearised), minicircle DNA (linear or supercoiled), plasmid DNA containing just the regions of interest by removal of the plasmid backbone by restriction enzyme digestion and purification, DNA generated using an enzymatic DNA amplification platform e.g. doggybone DNA (dbDNA™) where the final DNA used is in a closed ligated form or where it has been prepared (e.g restriction enzyme digestion) to have open cut ends.

A “viral vector production cell”, “vector production cell”, or “production cell” is to be understood as a cell that is capable of producing a viral vector or viral vector particle. Viral vector production cells may be “producer cells” or “packaging cells”. One or more DNA constructs of the viral vector system may be either stably integrated or episomally maintained within the viral vector production cell. Alternatively, all the DNA components of the viral vector system may be transiently transfected into the viral vector production cell. In yet another alternative, a production cell stably expressing some of the components may be transiently transfected with the remaining components required for vector production.

As used herein, the term “packaging cell” refers to a cell which contains the elements necessary for production of viral vector particles but which lacks the vector genome. Optionally, such packaging cells contain one or more expression cassettes which are capable of expressing viral structural proteins (such as gag, gag/pol and env).

Producer cells/packaging cells can be of any suitable cell type. Producer cells are generally mammalian cells but can be, for example, insect cells.

As used herein, the term “producer/production cell” or “vector producing/production cell” refers to a cell which contains all the elements necessary for production of viral vector particles. The producer cell may be either a stable producer cell line or derived transiently or may be a stable packaging cell wherein the viral genome is transiently expressed.

The vector production cells may be cells cultured in vitro such as a tissue culture cell line. Suitable cell lines include, but are not limited to, mammalian cells such as murine fibroblast derived cell lines or human cell lines. Preferably the vector production cells are derived from a human cell line.

Cells and Production Methods

The methods, viral vector production systems, and uses described herein are for producing a viral vector of interest.

General methods for producing viral vector from a cell (producer/production cell) comprising nucleic acid sequences encoding viral vector components are well known in the art. These methods comprise culturing the cell under conditions suitable for the production of the viral vectors, optionally with a further step of isolating the produced viral vector.

Suitable production cells or cells for producing a viral vector may be cells which are capable of producing viral vectors or viral vector particles when cultured under appropriate conditions. Thus, the cells typically comprise nucleotide sequences encoding vector components, which may include gag, env, rev and the genome of the viral vector. Suitable cell lines include, but are not limited to, mammalian cells such as murine fibroblast derived cell lines or human cell lines. They are generally mammalian, including human cells, for example HEK293T, HEK293, CAP, CAP-T or CHO cells, but can be, for example, insect cells such as SF9 cells. Preferably, the vector production cells are derived from a human cell line. Accordingly, such suitable production cells may be employed in any of the methods or uses of the present invention.

Methods for introducing nucleotide sequences into cells are well known in the art and have been described previously. Thus, the introduction into a cell of nucleotide sequences encoding vector components including gag, env, rev and the genome of the viral vector using conventional techniques in molecular and cell biology is within the capabilities of a person skilled in the art.

Stable production cells may be packaging or producer cells. To generate producer cells from packaging cells the vector genome DNA construct may be introduced stably or transiently. Packaging/producer cells can be generated by transducing a suitable cell line with a retroviral vector which expresses one of the components of the vector, i.e. a genome, the gag-pol components and an envelope as described in WO 2004/022761. Alternatively, the nucleotide sequence can be transfected into cells and then integration into the production cell genome occurs infrequently and randomly. The transfection methods may be performed using methods well known in the art. For example, a stable transfection process may employ constructs which have been engineered to aid concatemerisation. In another example, the transfection process may be performed using calcium phosphate or commercially available formulations such as Lipofectamine™ 2000 CD (Invitrogen, CA), FuGENE® HD or polyethylenimine (PEI). Alternatively nucleotide sequences may be introduced into the production cell via electroporation. The skilled person will be aware of methods to encourage integration of the nucleotide sequences into production cells. For example, linearising a nucleic acid construct can help if it is naturally circular. Less random integration methodologies may involve the nucleic acid construct comprising of areas of shared homology with the endogenous chromosomes of the mammalian host cell to guide integration to a selected site within the endogenous genome. Furthermore, if recombination sites are present on the construct then these can be used for targeted recombination. For example, the nucleic acid construct may contain a loxP site which allows for targeted integration when combined with Cre recombinase (i.e. using the Cre/lox system derived from P1 bacteriophage). Alternatively or additionally, the recombination site is an att site (e.g. from A phage), wherein the att site permits site-directed integration in the presence of a lambda integrase. This would allow the viral genes to be targeted to a locus within the host cellular genome which allows for high and/or stable expression.

Other methods of targeted integration are well known in the art. For example, methods of inducing targeted cleavage of genomic DNA can be used to encourage targeted recombination at a selected chromosomal locus. These methods often involve the use of methods or systems to induce a double strand break (DSB) e.g. a nick in the endogenous genome to induce repair of the break by physiological mechanisms such as non-homologous end joining (NHEJ). Cleavage can occur through the use of specific nucleases such as engineered zinc finger nucleases (ZFN), transcription-activator like effector nucleases (TALENs), using CRISPR/Cas9 systems with an engineered crRNA/tracr RNA (‘single guide RNA’) to guide specific cleavage, and/or using nucleases based on the Argonaute system (e.g., from T. thermophilus).

Packaging/producer cell lines can be generated by integration of nucleotide sequences using methods of just viral transduction or just nucleic acid transfection, or a combination of both can be used.

Methods for generating retroviral vectors from production cells and in particular the processing of retroviral vectors are described in WO 2009/153563.

In one example, the production cell may comprise the RNA-binding protein (e.g. tryptophan RNA-binding attenuation protein, TRAP) and/or the Tet Repressor (TetR) protein or alternative regulatory proteins (e.g. CymR).

Production of viral vector from production cells can be via transfection methods, from production from stable cell lines which can include induction steps (e.g. doxycycline induction) or via a combination of both. The transfection methods may be performed using methods well known in the art, and examples have been described previously.

Production cells, either packaging or producer cell lines or those transiently transfected with the viral vector encoding components are cultured to increase cell and virus numbers and/or virus titres. Culturing a cell is performed to enable it to metabolize, and/or grow and/or divide and/or produce viral vectors of interest. This can be accomplished by methods well known to persons skilled in the art, and includes but is not limited to providing nutrients for the cell, for instance in the appropriate culture media. The methods may comprise growth adhering to surfaces, growth in suspension, or combinations thereof. Culturing can be done for instance in tissue culture flasks, tissue culture multiwell plates, dishes, roller bottles, wave bags or in bioreactors, using batch, fed-batch, continuous systems and the like. In order to achieve large scale production of viral vector through cell culture it is preferred in the art to have cells capable of growing in suspension. Suitable conditions for culturing cells are known (see e.g. Tissue Culture, Academic Press, Kruse and Paterson, editors (1973), and R. I. Freshney, Culture of animal cells: A manual of basic technique, fourth edition (Wiley-Liss Inc., 2000, ISBN 0-471-34889-9).

Cells may initially be ‘bulked up’ in tissue culture flasks or bioreactors and subsequently grown in multi-layered culture vessels or large bioreactors (greater than 50 L) to generate the vector producing cells.

Cells may be grown in an adherent mode to generate the vector producing cells. Alternatively, cells may be grown in a suspension mode to generate the vector producing cells.

Nucleotide Sequences, Including Nucleotides of Interest (NOI)

As used herein, the term “nucleotide sequence” is synonymous with the term “polynucleotide” and/or the term “nucleic acid sequence”. The term “nucleotide sequence” in relation to the present invention can be a double stranded or single stranded molecule and includes genomic DNA, cDNA, synthetic DNA, RNA and a chimeric DNA/RNA molecule.

Typically, the nucleotide sequences encompassed by the scope of the present invention are prepared using recombinant DNA techniques (i.e. recombinant DNA). Such techniques are well known in the art.

Polynucleotides of the invention may comprise DNA or RNA. They may be single-stranded or double-stranded. It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed.

The polynucleotides may be modified by any method available in the art. Such modifications may be carried out in order to enhance the in vivo activity or lifespan of the polynucleotides of the invention.

Polynucleotides such as DNA polynucleotides may be produced recombinantly, synthetically or by any means available to those of skill in the art. They may also be cloned by standard techniques.

Longer polynucleotides will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA obtained from an animal or human cell, performing PCR under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

Common Viral Vector Elements

Promoters and Enhancers

Expression of a NOI and polynucleotide may be controlled using control sequences for example transcription regulation elements or translation repression elements, which include promoters, enhancers and other expression regulation signals (e.g. tet repressor (TetR) system) or the Transgene Repression In vector Production cell system (TRIP) or other regulators of NOIs described herein.

Prokaryotic promoters and promoters functional in eukaryotic cells may be used. Tissue-specific or stimuli-specific promoters may be used. Chimeric promoters may also be used comprising sequence elements from two or more different promoters.

Suitable promoting sequences are strong promoters including those derived from the genomes of viruses, such as polyoma virus, adenovirus, fowlpox virus, bovine papilloma virus, avian sarcoma virus, cytomegalovirus (CMV), retrovirus and Simian Virus 40 (SV40), or from heterologous mammalian promoters, such as the actin promoter, EF1a, CAG, TK, SV40, ubiquitin, PGK or ribosomal protein promoter. Alternatively, tissue-specific promoters such as rhodopsin (Rho), rhodopsin kinase (RhoK), cone-rod homeobox containing gene (CRX), neural retina-specific leucine zipper protein (NRL), Vitelliform Macular Dystrophy 2 (VMD2), Tyrosine hydroxylase, neuronal-specific neuronal-specific enolase (NSE) promoter, astrocyte-specific glial fibrillary acidic protein (GFAP) promoter, human al-antitrypsin (hAAT) promoter, phosphoenolpyruvate carboxykinase (PEPCK), liver fatty acid binding protein promoter, Flt-1 promoter, INF-β promoter, Mb promoter, SP-B promoter, SYN1 promoter, WASP promoter, SV40/hAIb promoter, SV40/CD43, SV40/CD45, NSE/RU5′ promoter, ICAM-2 promoter, GPIIb promoter, GFAP promoter, Fibronectin promoter, Endoglin promoter, Elastase-1 promoter, Desmin promoter, CD68 promoter, CD14 promoter and B29 promoter may be used to drive transcription.

Transcription of a NOI may be increased further by inserting an enhancer sequence into the vector. Enhancers are relatively orientation- and position-independent; however, one may employ an enhancer from a eukaryotic cell virus, such as the SV40 enhancer and the CMV early promoter enhancer. The enhancer may be spliced into the vector at a position 5′ or 3′ to the promoter, but is preferably located at a site 5′ from the promoter.

The promoter can additionally include features to ensure or to increase expression in a suitable target cell. For example, the features can be conserved regions e.g. a Pribnow Box or a TATA box. The promoter may contain other sequences to affect (such as to maintain, enhance or decrease) the levels of expression of a nucleotide sequence. Suitable other sequences include the Sh1-intron or an ADH intron. Other sequences include inducible elements, such as temperature, chemical, light or stress inducible elements. Also, suitable elements to enhance transcription or translation may be present.

Regulators of NOIs

A complicating factor in the generation of retroviral packaging/producer cell lines and retroviral vector production is that constitutive expression of certain retroviral vector components and NOIs are cytotoxic leading to death of cells expressing these components and therefore inability to produce vector. Therefore, the expression of these components (e.g. gag-pol and envelope proteins such as VSV-G) can be regulated. The expression of other non-cytotoxic vector components, e.g. rev, can also be regulated to minimise the metabolic burden on the cell. Thus the modular constructs or nucleotide sequences encoding vector components and/or cells as described herein may comprise cytotoxic and/or non-cytotoxic vector components associated with at least one regulatory element. As used herein, the term “regulatory element” refers to any element capable of affecting, either increasing or decreasing, the expression of an associated gene or protein. A regulatory element includes a gene switch system, transcription regulation element and translation repression element

A number of prokaryotic regulator systems have been adapted to generate gene switches in mammalian cells. Many retroviral packaging and producer cell lines have been controlled using gene switch systems (e.g. tetracycline and cumate inducible switch systems) thus enabling expression of one or more of the retroviral vector components to be switched on at the time of vector production. Gene switch systems include those of the (TetR) protein group of transcription regulators (e.g. T-Rex, Tet-On, and Tet-Off), those of the cumate inducible switch system group of transcription regulators (e.g. CymR protein) and those involving an RNA-binding protein (e.g. TRAP).

One such tetracycline-inducible system is the tetracycline repressor (TetR) system based on the T-REx™ system. By way of example, in such a system tetracycline operators (TetO₂) are placed in a position such that the first nucleotide is 10 bp from the 3′ end of the last nucleotide of the TATATAA element of the human cytomegalovirus major immediate early promoter (hCMVp) then TetR alone is capable of acting as a repressor (Yao F, Svensjo T, Winkler T, Lu M, Eriksson C, Eriksson E., 1998, Hum Gene Ther, 9: 1939-1950). In such a system the expression of the NOI can be controlled by a CMV promoter into which two copies of the TetO₂ sequence have been inserted in tandem. TetR homodimers, in the absence of an inducing agent (tetracycline or its analogue doxycycline [dox]), bind to the TetO₂ sequences and physically block transcription from the upstream CMV promoter. When present, the inducing agent binds to the TetR homodimers, causing allosteric changes such that it can no longer bind to the TetO₂ sequences, resulting in gene expression. The TetR gene may be codon optimised as this was found to improve translation efficiency resulting in tighter control of TetO₂ controlled gene expression.

The TRIP system is described in WO 2015/092440 and provides another way of repressing expression of the NOI in the production cells during vector production. The TRAP-binding sequence (e.g. TRAP-tbs) interaction forms the basis for a transgene protein repression system for the production of retroviral vectors, when a constitutive and/or strong promoter, including a tissue-specific promoter, driving the transgene is desirable and particularly when expression of the transgene protein in production cells leads to reduction in vector titres and/or elicits an immune response in vivo due to viral vector delivery of transgene-derived protein (Maunder et al, Nat Commun. (2017) March 27; 8).

Briefly, the TRAP-tbs interaction forms a translational block, repressing translation of the transgene protein (Maunder et al, Nat Commun. (2017) March 27; 8). The translational block is only effective in production cells and as such does not impede the DNA- or RNA-based vector systems. The TRiP system is able to repress translation when the transgene protein is expressed from a constitutive and/or strong promoter, including a tissue-specific promoter from single- or multi cistronic mRNA. It has been demonstrated that unregulated expression of transgene protein can reduce vector titres and affect vector product quality. Repression of transgene protein for both transient and stable PaCL/PCL vector production systems is beneficial for production cells to prevent a reduction in vector titres: where toxicity or molecular burden issues may lead to cellular stress; where transgene protein elicits an immune response in vivo due to viral vector delivery of transgene-derived protein; where the use of gene-editing transgenes may result in on/off target affects; where the transgene protein may affect vector and/or envelope glycoprotein exclusion.

Envelope and Pseudotyping

In one preferred aspect, the lentiviral vector as described herein has been pseudotyped. In this regard, pseudotyping can confer one or more advantages. For example, the env gene product of the HIV based vectors would restrict these vectors to infecting only cells that express a protein called CD4. But if the env gene in these vectors has been substituted with env sequences from other enveloped viruses, then they may have a broader infectious spectrum (Verma and Somia (1997) Nature 389(6648):239-242). By way of example, workers have pseudotyped an HIV based vector with the glycoprotein from VSV (Verma and Somia (1997) Nature 389(6648):239-242).

In another alternative, the Env protein may be a modified Env protein such as a mutant or engineered Env protein. Modifications may be made or selected to introduce targeting ability or to reduce toxicity or for another purpose (Valsesia-Wittman et al 1996 J Virol 70: 2056-64; Nilson et al (1996) Gene Ther 3(4):280-286; and Fielding et al (1998) Blood 91(5):1802-1809 and references cited therein).

The vector may be pseudotyped with any molecule of choice.

As used herein, “env” shall mean an endogenous lentiviral envelope or a heterologous envelope, as described herein.

VSV-G

The envelope glycoprotein (G) of Vesicular stomatitis virus (VSV), a rhabdovirus, is an envelope protein that has been shown to be capable of pseudotyping certain enveloped viruses and viral vector virions.

Its ability to pseudotype MoMLV-based retroviral vectors in the absence of any retroviral envelope proteins was first shown by Emi et al. (1991) Journal of Virology 65:1202-1207. WO 1994/294440 teaches that retroviral vectors may be successfully pseudotyped with VSV-G. These pseudotyped VSV-G vectors may be used to transduce a wide range of mammalian cells. More recently, Abe et al. (1998) J Virol 72(8) 6356-6361 teach that non-infectious retroviral particles can be made infectious by the addition of VSV-G.

Burns et al. (1993) Proc. Natl. Acad. Sci. USA 90:8033-7 successfully pseudotyped the retrovirus MLV with VSV-G and this resulted in a vector having an altered host range compared to MLV in its native form. VSV-G pseudotyped vectors have been shown to infect not only mammalian cells, but also cell lines derived from fish, reptiles and insects (Burns et al. (1993) ibid). They have also been shown to be more efficient than traditional amphotropic envelopes for a variety of cell lines (Yee et al., (1994) Proc. Natl. Acad. Sci. USA 91:9564-9568, Emi et al. (1991) Journal of Virology 65:1202-1207). VSV-G protein can be used to pseudotype certain retroviruses because its cytoplasmic tail is capable of interacting with the retroviral cores.

The provision of a non-retroviral pseudotyping envelope such as VSV-G protein gives the advantage that vector particles can be concentrated to a high titre without loss of infectivity (Akkina et al. (1996) J. Virol. 70:2581-5). Retrovirus envelope proteins are apparently unable to withstand the shearing forces during ultracentrifugation, probably because they consist of two non-covalently linked subunits. The interaction between the subunits may be disrupted by the centrifugation. In comparison the VSV glycoprotein is composed of a single unit. VSV-G protein pseudotyping can therefore offer potential advantages for both efficient target cell infection/transduction and during manufacturing processes.

WO 2000/52188 describes the generation of pseudotyped retroviral vectors, from stable producer cell lines, having vesicular stomatitis virus-G protein (VSV-G) as the membrane-associated viral envelope protein, and provides a gene sequence for the VSV-G protein.

Ross River Virus

The Ross River viral envelope has been used to pseudotype a non-primate lentiviral vector (FIV) and following systemic administration predominantly transduced the liver (Kang et al., 2002, J. Virol., 76:9378-9388). Efficiency was reported to be 20-fold greater than obtained with VSV-G pseudotyped vector, and caused less cytotoxicity as measured by serum levels of liver enzymes suggestive of hepatotoxicity.

Baculovirus GP64

The baculovirus GP64 protein has been shown to be an alternative to VSV-G for viral vectors used in the large-scale production of high-titre virus required for clinical and commercial applications (Kumar M, Bradow B P, Zimmerberg J (2003) Hum Gene Ther. 14(1):67-77). Compared with VSV-G-pseudotyped vectors, GP64-pseudotyped vectors have a similar broad tropism and similar native titres. Because, GP64 expression does not kill cells, HEK293T-based cell lines constitutively expressing GP64 can be generated.

Alternative Envelopes

Other envelopes which give reasonable titre when used to pseudotype EIAV include Mokola, Rabies, Ebola and LCMV (lymphocytic choriomeningitis virus). Intravenous infusion into mice of lentivirus pseudotyped with 4070A led to maximal gene expression in the liver.

Packaging Sequence

As utilized within the context of the present invention the term “packaging signal”, which is referred to interchangeably as “packaging sequence” or “psi”, is used in reference to the non-coding, cis-acting sequence required for encapsidation of retroviral RNA strands during viral particle formation. In HIV-1, this sequence has been mapped to loci extending from upstream of the major splice donor site (SD) to at least the gag start codon (some or all of the 5′ sequence of gag to nucleotide 688 may be included). In EIAV the packaging signal comprises the R region into the 5′ coding region of Gag.

As used herein, the term “extended packaging signal” or “extended packaging sequence” refers to the use of sequences around the psi sequence with further extension into the gag gene. The inclusion of these additional packaging sequences may increase the efficiency of insertion of vector RNA into viral particles.

Feline immunodeficiency virus (FIV) RNA encapsidation determinants have been shown to be discrete and non-continuous, comprising one region at the 5′ end of the genomic mRNA (R-U5) and another region that mapped within the proximal 311 nt of gag (Kaye et al., J Virol. October; 69(10):6588-92 (1995).

Internal Ribosome Entry Site (IRES)

Insertion of IRES elements allows expression of multiple coding regions from a single promoter (Adam et al (as above); Koo et al (1992) Virology 186:669-675; Chen et al 1993 J. Virol 67:2142-2148). IRES elements were first found in the non-translated 5′ ends of picornaviruses where they promote cap-independent translation of viral proteins (Jang et al (1990) Enzyme 44: 292-309). When located between open reading frames in an RNA, IRES elements allow efficient translation of the downstream open reading frame by promoting entry of the ribosome at the IRES element followed by downstream initiation of translation. A review on IRES is presented by Mountford and Smith (TIG May 1995 vol 11, No 5:179-184). A number of different IRES sequences are known including those from encephalomyocarditis virus (EMCV) (Ghattas, I. R., et al., Mol. Cell. Biol., 11:5848-5859 (1991); BiP protein [Macejak and Sarnow, Nature 353:91 (1991)]; the Antennapedia gene of Drosophila (exons d and e) [Oh, et al., Genes & Development, 6:1643-1653 (1992)] as well as those in polio virus (PV) [Pelletier and Sonenberg, Nature 334: 320-325 (1988); see also Mountford and Smith, TIG 11, 179-184 (1985)].

IRES elements from PV, EMCV and swine vesicular disease virus have previously been used in retroviral vectors (Coffin et al, as above).

The term “IRES” includes any sequence or combination of sequences which work as or improve the function of an IRES. The IRES(s) may be of viral origin (such as EMCV IRES, PV IRES, or FMDV 2A-like sequences) or cellular origin (such as FGF2 IRES, NRF IRES, Notch 2 IRES or EIF4 IRES).

In order for the IRES to be capable of initiating translation of each polynucleotide it should be located between or prior to the polynucleotides in the modular construct.

The nucleotide sequences utilised for development of stable cell lines require the addition of selectable markers for selection of cells where stable integration has occurred. These selectable markers can be expressed as a single transcription unit within the nucleotide sequence or it may be preferable to use IRES elements to initiate translation of the selectable marker in a polycistronic message (Adam et al 1991 J. Virol. 65, 4985).

Genetic Orientation and Insulators

It is well known that nucleic acids are directional and this ultimately affects mechanisms such as transcription and replication in the cell. Thus genes can have relative orientations with respect to one another when part of the same nucleic acid construct.

In certain examples, at least two nucleic acid sequences present at the same locus in the cell or construct can be in a reverse and/or alternating orientations. In other words, at this particular locus, the pair of sequential genes will not have the same orientation. This can help prevent both transcriptional and translational read-through when the region is expressed within the same physical location of the host cell.

Having the alternating orientations benefits viral vector production when the nucleic acids required for vector production are based at the same genetic locus within the cell. This in turn can also improve the safety of the resulting constructs in preventing the generation of replication-competent viral vectors.

When nucleic acid sequences are in reverse and/or alternating orientations the use of insulators can prevent inappropriate expression or silencing of a NOI from its genetic surroundings.

The term “insulator” refers to a class of DNA sequence elements that when bound to insulator-binding proteins possess an ability to protect genes from surrounding regulator signals. There are two types of insulators: an enhancer blocking function and a chromatin barrier function. When an insulator is situated between a promoter and an enhancer, the enhancer-blocking function of the insulator shields the promoter from the transcription-enhancing influence of the enhancer (Geyer and Corces 1992; Kellum and Schedl 1992). The chromatin barrier insulators function by preventing the advance of nearby condensed chromatin which would lead to a transcriptionally active chromatin region turning into a transcriptionally inactive chromatin region and resulting in silencing of gene expression. Insulators which inhibit the spread of heterochromatin, and thus gene silencing, recruit enzymes involved in histone modifications to prevent this process (Yang J, Corces V G. 2011; 110:43-76; Huang, Li et al. 2007; Dhillon, Raab et al. 2009). An insulator can have one or both of these functions and the chicken β-globin insulator (cHS4) is one such example. This insulator is the most extensively studied vertebrate insulator, is highly rich in G+C and has both enhancer-blocking and heterochromatic barrier functions (Chung J H, Whitely M, Felsenfeld G. Cell. 1993; 74:505-514). Other such insulators with enhancer blocking functions are not limited to but include the following: human β-globin insulator 5 (HS5), human β-globin insulator 1 (HS1), and chicken β-globin insulator (cHS3) (Farrell CM1, West AG, Felsenfeld G., Mol Cell Biol. 2002 June; 22(11):3820-31; J Ellis et al. EMBO J. 1996 Feb. 1; 15(3): 562-568). In addition to reducing unwanted distal interactions the insulators also help to prevent promoter interference (i.e. where the promoter from one transcription unit impairs expression of an adjacent transcription unit) between adjacent viral nucleic acid sequences. If the insulators are used between each of the viral vector nucleic acid sequences, then the reduction of direct read-through will help prevent the formation of replication-competent viral vector particles.

The insulator may be present between each of the viral nucleic acid sequences. In one embodiment, the use of insulators prevents promoter-enhancer interactions from one NOI expression cassette interacting with another NOI expression cassette in a nucleotide sequence encoding vector components.

An insulator may be present between the vector genome and gag-pol sequences. This therefore limits the likelihood of the production of a replication-competent viral vector and ‘wild-type’ like RNA transcripts, improving the safety profile of the construct. The use of insulator elements to improve the expression of stably integrated multigene vectors is cited in Moriarity et al, Nucleic Acids Res. 2013 April; 41(8):e92.

Vector Titre

The skilled person will understand that there are a number of different methods of determining the titre of viral vectors (e.g. the viral titre of lentiviral vectors, SIN vectors). Titre is often described as transducing units/mL (TU/mL). Titre may be increased by increasing the number of vector particles and by increasing the specific activity of a vector preparation.

Therapeutic Use

The viral vector as described herein or a cell or tissue transduced with the viral vector as described herein may be used in medicine.

In addition, the viral vector as described herein, a production cell or a cell or tissue transduced with the lentiviral vector as described herein may be used for the preparation of a medicament to deliver a nucleotide of interest to a target site in need of the same. Such uses of the viral vector or transduced cell may be for therapeutic or diagnostic purposes, as described previously.

Accordingly, there is provided a cell transduced by the viral vector as described herein.

A “cell transduced by a viral vector particle” is to be understood as a cell, in particular a target cell, into which the nucleic acid carried by the viral vector particle has been transferred.

Nucleotide of Interest

In one embodiment of the invention, the nucleotide of interest is translated in a target cell which lacks TRAP.

“Target cell” is to be understood as a cell in which it is desired to express the NOI. The NOI may be introduced into the target cell using a viral vector of the present invention. Delivery to the target cell may be performed in vivo, ex vivo or in vitro.

In a preferred embodiment, the nucleotide of interest gives rise to a therapeutic effect.

The NOI may have a therapeutic or diagnostic application. Suitable NOIs include, but are not limited to sequences encoding enzymes, co-factors, cytokines, chemokines, hormones, antibodies, anti-oxidant molecules, engineered immunoglobulin-like molecules, single chain antibodies, fusion proteins, immune co-stimulatory molecules, immunomodulatory molecules, chimeric antigen receptors a transdomain negative mutant of a target protein, toxins, conditional toxins, antigens, transcription factors, structural proteins, reporter proteins, subcellular localization signals, tumour suppressor proteins, growth factors, membrane proteins, receptors, vasoactive proteins and peptides, anti-viral proteins and ribozymes, and derivatives thereof (such as derivatives with an associated reporter group). The NOIs may also encode micro-RNA. Without wishing to be bound by theory, it is believed that the processing of micro-RNA will be inhibited by TRAP.

In one embodiment, the NOI may be useful in the treatment of a neurodegenerative disorder.

In another embodiment, the NOI may be useful in the treatment of Parkinson's disease and multiple system atrophy.

In another embodiment, the NOI may encode an enzyme or enzymes involved in dopamine synthesis. For example, the enzyme may be one or more of the following: tyrosine hydroxylase, GTP-cyclohydrolase I and/or aromatic amino acid dopa decarboxylase. The sequences of all three genes are available (GenBank® Accession Nos. X05290, U19523 and M76180, respectively).

In another embodiment, the NOI may encode the vesicular monoamine transporter 2 (VMAT2). In an alternative embodiment the viral genome may comprise a NOI encoding aromatic amino acid dopa decarboxylase and a NOI encoding VMAT2. Such a genome may be used in the treatment of Parkinson's disease, in particular in conjunction with peripheral administration of L-DOPA.

In another embodiment the NOI may encode a therapeutic protein or combination of therapeutic proteins.

In another embodiment, the NOI may encode a protein or proteins selected from the group consisting of glial cell derived neurotophic factor (GDNF), brain derived neurotrophic factor (BDNF), ciliary neurotrophic factor (CNTF), neurotrophin-3 (NT-3), acidic fibroblast growth factor (aFGF), basic fibroblast growth factor (bFGF), interleukin-1 beta (IL-1β), tumor necrosis factor alpha (TNF-α), insulin growth factor-2, VEGF-A, VEGF-B, VEGF-C/VEGF-2, VEGF-D, VEGF-E, PDGF-A, PDGF-B, hetero- and homo-dimers of PDFG-A and PDFG-B.

In another embodiment, the NOI may encode an anti-angiogenic protein or anti-angiogenic proteins selected from the group consisting of angiostatin, endostatin, platelet factor 4, pigment epithelium derived factor (PEDF), placental growth factor, restin, interferon-α, interferon-inducible protein, gro-beta and tubedown-1, interleukin(IL)-1, IL-12, retinoic acid, anti-VEGF antibodies or fragments/variants thereof such as aflibercept, thrombospondin, VEGF receptor proteins such as those described in U.S. Pat. Nos. 5,952,199 and 6,100,071, and anti-VEGF receptor antibodies.

In another embodiment, the NOI may encode anti-inflammatory proteins, antibodies or fragment/variants of proteins or antibodies selected from the group consisting of NF-kB inhibitors, IL1beta inhibitors, TGFbeta inhibitors, IL-6 inhibitors, IL-23 inhibitors, IL-18 inhibitors, Tumour necrosis factor alpha and Tumour necrosis factor beta, Lymphotoxin alpha and Lymphotoxin beta, LIGHT inhibitors, alpha synuclein inhibitors, Tau inhibitors, beta amyloid inhibitors, IL-17 inhibitors, IL-33 inhibitors, IL-33 receptor inhibitors, TSLP inhibitors.

In another embodiment the NOI may encode cystic fibrosis transmembrane conductance regulator (CFTR).

In another embodiment the NOI may encode a protein normally expressed in an ocular cell.

In another embodiment, the NOI may encode a protein normally expressed in a photoreceptor cell and/or retinal pigment epithelium cell.

In another embodiment, the NOI may encode a protein selected from the group comprising RPE65, arylhydrocarbon-interacting receptor protein like 1 (AIPL1), CRB1, lecithin retinal acetyltransferace (LRAT), photoreceptor-specific homeo box (CRX), retinal guanylate cyclise (GUCY2D), RPGR interacting protein 1 (RPGRIP1), LCA2, LCA3, LCA5, dystrophin, PRPH2, CNTF, ABCR/ABCA4, EMP1, TIMP3, MERTK, ELOVL4, MYO7A, USH2A, VMD2, RLBP1, COX-2, FPR, harmonin, Rab escort protein 1, CNGB2, CNGA3, CEP 290, RPGR, RS1, RP1, PRELP, glutathione pathway enzymes and opticin.

In other embodiments, the NOI may encode the human clotting Factor VIII or Factor IX.

In other embodiments, the NOI may encode protein or proteins involved in metabolism selected from the group comprising phenylalanine hydroxylase (PAH), Methylmalonyl CoA mutase, Propionyl CoA carboxylase, Isovaleryl CoA dehydrogenase, Branched chain ketoacid dehydrogenase complex, Glutaryl CoA dehydrogenase, Acetyl CoA carboxylase, propionyl CoA carboxylase, 3 methyl crotonyl CoA carboxylase, pyruvate carboxylase, carbamoyl-phophate synthase ammonia, ornithine transcarbamylase, alpha galactosidase A, glucosylceramidase beta, cystinosin, glucosamine(N-acetyl)-6-sulfatase, N-acetyl-alpha-glucosaminidase, glucose-6-phosphatase, ATP7B, ATP8B1, ABCB11, ABCB4, TJP2, N-sulfoglucosamine sulfohydrolase, Galactosamine-6 sulfatase, arylsulfatase A, cytochrome 8-245 beta, ABCD1, ornithine carbamoyltransferase, argininosuccinate synthase, argininosuccinate lysase, arginase 1, alanine glycoxhylate amino transferase, ATP-binding cassette, sub-family B members.

In other embodiments, the NOI may encode a chimeric antigen receptor (CAR) or a T cell receptor (TCR). In one embodiment, the CAR is an anti-5T4 CAR. In other embodiments, the NOI may encode B-cell maturation antigen (BCMA), CD19, CD22, CD20, CD138, CD30, CD33, CD123, CD70, prostate specific membrane antigen (PSMA), Lewis Y antigen (LeY), Tyrosine-protein kinase transmembrane receptor (ROR1), Mucin 1, cell surface associated (Muc1), Epithelial cell adhesion molecule (EpCAM), endothelial growth factor receptor (EGFR), insulin, protein tyrosine phosphatase, non-receptor type 22, interleukin 2 receptor, alpha, interferon induced with helicase C domain 1, human epidermal growth factor receptor (HER2), glypican 3 (GPC3), disialoganglioside (GD2), mesothelin, vesicular endothelial growth factor receptor 2 (VEGFR2), Smith antigen, double stranded DNA, phospholipids, proinsulin, insulinoma antigen 2 (IA-2), 65 kDa isoform of glutamic add decarboxylase (GAD65), chromogranin A (CHGA), islet amyloid polypeptide (IAPP), islet-specific glucose-6-phosphatase catalytic subunit-reiated protein (IGRP), zinc transporter 8 (ZnT8).

In other embodiments, the NOI may encode a chimeric antigen receptor (CAR) against NKG2D ligands selected from the group comprising ULBP1, 2 and 3, H60, Rae-1a, b, g, d, MICA, MICB.

In further embodiments the NOI may encode SGSH, SUMF1, GAA, the common gamma chain (CD132), adenosine deaminase, WAS protein, globins, alpha galactosidase A, δ-aminolevulinate (ALA) synthase, δ-aminolevulinate dehydratase (ALAD), Hydroxymethylbilane (HMB) synthase, Uroporphyrinogen (URO) synthase, Uroporphyrinogen (URO) decarboxylase, Coproporphyrinogen (COPRO) oxidase, Protoporphyrinogen (PROTO) oxidase, Ferrochelatase, α-L-iduronidase, Iduronate sulfatase, Heparan sulfamidase, N-acetylglucosaminidase, Heparan-α-glucosaminide N-acetyltransferase, 3 N-acetylglucosamine 6-sulfatase, Galactose-6-sulfate sulfatase, β-galactosidase, N-acetylgalactosamine-4-sulfatase, β-glucuronidase and Hyaluronidase.

In addition to the NOI the vector may also comprise or encode a siRNA, shRNA, or regulated shRNA. (Dickins et al. (2005) Nature Genetics 37: 1289-1295, Silva et al. (2005) Nature Genetics 37:1281-1288).

Indications

The vectors, including retroviral and AAV vectors, according to the present invention may be used to deliver one or more NOI(s) useful in the treatment of the disorders listed in WO 1998/05635, WO 1998/07859, WO 1998/09985. The nucleotide of interest may be DNA or RNA. Examples of such diseases are given below:
A disorder which responds to cytokine and cell proliferation/differentiation activity; immunosuppressant or immunostimulant activity (e.g. for treating immune deficiency, including infection with human immunodeficiency virus, regulation of lymphocyte growth; treating cancer and many autoimmune diseases, and to prevent transplant rejection or induce tumour immunity); regulation of haematopoiesis (e.g. treatment of myeloid or lymphoid diseases); promoting growth of bone, cartilage, tendon, ligament and nerve tissue (e.g. for healing wounds, treatment of burns, ulcers and periodontal disease and neurodegeneration); inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); anti-inflammatory activity (for treating, for example, septic shock or Crohn's disease); macrophage inhibitory and/or T cell inhibitory activity and thus, anti-inflammatory activity; anti-immune activity (i.e. inhibitory effects against a cellular and/or humoral immune response, including a response not associated with inflammation); inhibition of the ability of macrophages and T cells to adhere to extracellular matrix components and fibronectin, as well as up-regulated fas receptor expression in T cells.
Malignancy disorders, including cancer, leukaemia, benign and malignant tumour growth, invasion and spread, angiogenesis, metastases, ascites and malignant pleural effusion.
Autoimmune diseases including arthritis, including rheumatoid arthritis, hypersensitivity, psoriasis, Sjogren's syndrome, allergic reactions, asthma, systemic lupus erythematosus, Type 1 diabetes mellitus, collagen diseases and other diseases.
Vascular diseases including arteriosclerosis, atherosclerotic heart disease, reperfusion injury, cardiac arrest, myocardial infarction, vascular inflammatory disorders, respiratory distress syndrome, cardiovascular effects, peripheral vascular disease, migraine and aspirin-dependent anti-thrombosis, stroke, cerebral ischaemia, ischaemic heart disease or other diseases.
Diseases of the gastrointestinal tract including peptic ulcer, ulcerative colitis, Crohn's disease and other diseases.
Hepatic diseases including hepatic fibrosis, liver cirrhosis, amyloidosis.
Inherited metabolic disorders including phenylketonuria PKU, Wilson disease, organic acidemias, glycogen storage diseases, urea cycle disorders, cholestasis, and other diseases, or other diseases.
Renal and urologic diseases including thyroiditis or other glandular diseases, glomerulonephritis, lupus nephritis or other diseases.
Ear, nose and throat disorders including otitis or other oto-rhino-laryngological diseases, dermatitis or other dermal diseases.
Dental and oral disorders including periodontal diseases, periodontitis, gingivitis or other dental/oral diseases.
Testicular diseases including orchitis or epididimo-orchitis, infertility, orchidal trauma or other testicular diseases.
Gynaecological diseases including placental dysfunction, placental insufficiency, habitual abortion, eclampsia, pre-eclampsia, endometriosis and other gynaecological diseases.
Ophthalmologic disorders such as Leber Congenital Amaurosis (LCA) including LCA10, posterior uveitis, intermediate uveitis, anterior uveitis, conjunctivitis, chorioretinitis, uveoretinitis, optic neuritis, glaucoma, including open angle glaucoma and juvenile congenital glaucoma, intraocular inflammation, e.g. retinitis or cystoid macular oedema, sympathetic ophthalmia, scleritis, retinitis pigmentosa, macular degeneration including age related macular degeneration (AMD) and juvenile macular degeneration including Best Disease, Best vitelliform macular degeneration, Stargardt's Disease, Usher's syndrome, Doyne's honeycomb retinal dystrophy, Sorby's Macular Dystrophy, Juvenile retinoschisis, Cone-Rod Dystrophy, Corneal Dystrophy, Fuch's Dystrophy, Leber's congenital amaurosis, Leber's hereditary optic neuropathy (LHON), Adie syndrome, Oguchi disease, degenerative fondus disease, ocular trauma, ocular inflammation caused by infection, proliferative vitreo-retinopathies, acute ischaemic optic neuropathy, excessive scarring, e.g. following glaucoma filtration operation, reaction against ocular implants, corneal transplant graft rejection, and other ophthalmic diseases, such as diabetic macular oedema, retinal vein occlusion, RLBP1-associated retinal dystrophy, choroideremia and achromatopsia.
Neurological and neurodegenerative disorders including Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, strokes, post-polio syndrome, psychiatric disorders, myelitis, encephalitis, subacute sclerosing pan-encephalitis, encephalomyelitis, acute neuropathy, subacute neuropathy, chronic neuropathy, Fabry disease, Gaucher disease, Cystinosis, Pompe disease, metachromatic leukodystrophy, Wscott Aldrich Syndrome, adrenoleukodystrophy, beta-thalassemia, sickle cell disease, Guillaim-Barre syndrome, Sydenham chorea, myasthenia gravis, pseudo-tumour cerebri, Down's Syndrome, Huntington's disease, CNS compression or CNS trauma or infections of the CNS, muscular atrophies and dystrophies, diseases, conditions or disorders of the central and peripheral nervous systems, motor neuron disease including amyotropic lateral sclerosis, spinal muscular atropy, spinal cord and avulsion injury.
Other diseases and conditions such as cystic fibrosis, mucopolysaccharidosis including Sanfilipo syndrome A, Sanfilipo syndrome B, Sanfilipo syndrome C, Sanfilipo syndrome D, Hunter syndrome, Hurler-Scheie syndrome, Morquio syndrome, ADA-SCID, X-linked SCID, X-linked chronic granulomatous disease, porphyria, haemophilia A, haemophilia B, post-traumatic inflammation, haemorrhage, coagulation and acute phase response, cachexia, anorexia, acute infection, septic shock, infectious diseases, diabetes mellitus, complications or side effects of surgery, bone marrow transplantation or other transplantation complications and/or side effects, complications and side effects of gene therapy, e.g. due to infection with a viral carrier, or AIDS, to suppress or inhibit a humoral and/or cellular immune response, for the prevention and/or treatment of graft rejection in cases of transplantation of natural or artificial cells, tissue and organs such as cornea, bone marrow, organs, lenses, pacemakers, natural or artificial skin tissue.

siRNA, micro-RNA and shRNA

In certain other embodiments, the NOI comprises a micro-RNA. Micro-RNAs are a very large group of small RNAs produced naturally in organisms, at least some of which regulate the expression of target genes. Founding members of the micro-RNA family are let-7 and lin-4. The let-7 gene encodes a small, highly conserved RNA species that regulates the expression of endogenous protein-coding genes during worm development. The active RNA species is transcribed initially as an ˜70 nt precursor, which is post-transcriptionally processed into a mature ˜21 nt form. Both let-7 and lin-4 are transcribed as hairpin RNA precursors which are processed to their mature forms by Dicer enzyme.

In addition to the NOI the vector may also comprise or encode a siRNA, shRNA, or regulated shRNA (Dickins et al. (2005) Nature Genetics 37: 1289-1295, Silva et al. (2005) Nature Genetics 37:1281-1288).

Post-transcriptional gene silencing (PTGS) mediated by double-stranded RNA (dsRNA) is a conserved cellular defence mechanism for controlling the expression of foreign genes. It is thought that the random integration of elements such as transposons or viruses causes the expression of dsRNA which activates sequence-specific degradation of homologous single-stranded mRNA or viral genomic RNA. The silencing effect is known as RNA interference (RNAi) (Ralph et al. (2005) Nature Medicine 11:429-433). The mechanism of RNAi involves the processing of long dsRNAs into duplexes of about 21-25 nucleotide (nt) RNAs. These products are called small interfering or silencing RNAs (siRNAs) which are the sequence-specific mediators of mRNA degradation. In differentiated mammalian cells, dsRNA >30 bp has been found to activate the interferon response leading to shut-down of protein synthesis and non-specific mRNA degradation (Stark et al., Annu Rev Biochem 67:227-64 (1998)). However this response can be bypassed by using 21 nt siRNA duplexes (Elbashir et al., EMBO J. Dec. 3; 20(23):6877-88 (2001), Hutvagner et al., Science. August 3, 293(5531):834-8. Eupub July 12 (2001)) allowing gene function to be analysed in cultured mammalian cells.

Pharmaceutical Compositions

A pharmaceutical composition is provided comprising the viral vector as described herein or a cell or tissue transduced with the viral vector as described herein, in combination with a pharmaceutically acceptable carrier, diluent or excipient.

A pharmaceutical composition for treating an individual by gene therapy is provided, wherein the composition comprises a therapeutically effective amount of a viral vector. The pharmaceutical composition may be for human or animal usage.

The composition may comprise a pharmaceutically acceptable carrier, diluent, excipient or adjuvant. The choice of pharmaceutical carrier, excipient or diluent can be made with regard to the intended route of administration and standard pharmaceutical practice. The pharmaceutical compositions may comprise, or be in addition to, the carrier, excipient or diluent any suitable binder(s), lubricant(s), suspending agent(s), coating agent(s), solubilising agent(s) and other carrier agents that may aid or increase vector entry into the target site (such as for example a lipid delivery system).

Where appropriate, the composition can be administered by any one or more of inhalation; in the form of a suppository or pessary; topically in the form of a lotion, solution, cream, ointment or dusting powder; by use of a skin patch; orally in the form of tablets containing excipients such as starch or lactose, or in capsules or ovules either alone or in admixture with excipients, or in the form of elixirs, solutions or suspensions containing flavouring or colouring agents; or they can be injected parenterally, for example intracavernosally, intravenously, intramuscularly, intracranially, intraoccularly intraperitoneally, or subcutaneously. For parenteral administration, the compositions may be best used in the form of a sterile aqueous solution which may contain other substances, for example enough salts or monosaccharides to make the solution isotonic with blood. For buccal or sublingual administration, the compositions may be administered in the form of tablets or lozenges which can be formulated in a conventional manner.

The viral vector as described herein may also be used to transduce target cells or target tissue ex vivo prior to transfer of said target cell or tissue into a patient in need of the same. An example of such cell may be autologous T cells and an example of such tissue may be a donor cornea.

Variants, Derivatives, Analogues, Homologues and Fragments

In addition to the specific proteins and nucleotides mentioned herein, the present invention also encompasses the use of variants, derivatives, pharmaceutically acceptable salts, analogues, homologues and fragments thereof.

A variant of any given sequence is a sequence in which the specific sequence of residues (whether amino acid or nucleic acid residues) has been modified in such a manner that the polypeptide or polynucleotide in question retains at least one of its endogenous functions. A variant sequence can be obtained by addition, deletion, substitution, modification, replacement and/or variation of at least one residue present in the naturally-occurring protein.

The term “derivative” as used herein, in relation to proteins or polypeptides includes any substitution of, variation of, modification of, replacement of, deletion of and/or addition of one (or more) amino acid residues from or to the sequence providing that the resultant protein or polypeptide retains at least one of its endogenous functions.

The term “analogue” as used herein, in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3 to 10 or 20 substitutions provided that the modified sequence retains the required activity or ability. Amino acid substitutions may include the use of non-naturally occurring analogues. Proteins used herein may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R H |
| AROMATIC | | F WY |

The term “homologue” means an entity having a certain homology with the wild type amino acid sequence and the wild type nucleotide sequence. The term “homology” can be equated with “identity”.

In the present context, a homologous sequence is taken to include an amino acid sequence which may be at least 50%, 55%, 65%, 75%, 85% or 90% identical, preferably at least 95%, 97 or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, a homologous sequence is taken to include a nucleotide sequence which may be at least 50%, 55%, 65%, 75%, 85% or 90% identical, preferably at least 95%, 97%, 98% or 99% identical to the subject sequence. Although homology can also be considered in terms of similarity, in the context of the present invention it is preferred to express homology in terms of sequence identity.

Homology comparisons can be conducted by eye, or more usually, with the aid of readily available sequence comparison programs. These commercially available computer programs can calculate percentage homology or identity between two or more sequences. Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is ˜12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Research 12:387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol Lett (1999) 174(2):247-50; FEMS Microbiol Lett (1999) 177(1):187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Once the software has produced an optimal alignment, it is possible to calculate percentage homology, preferably percentage sequence identity. The software usually does this as part of the sequence comparison and generates a numerical result. “Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the break. The DNA is then expressed in accordance with the invention to make the encoded protein. These methods are only illustrative of the numerous standard techniques known in the art for manipulation of DNA sequences and other known techniques may also be used.

All variants, fragments or homologues of the regulatory protein suitable for use in the cells and/or modular constructs of the invention will retain the ability to bind the cognate binding site of the NOI such that translation of the NOI is repressed or prevented in a viral vector production cell.

All variants fragments or homologues of the binding site will retain the ability to bind the cognate RNA-binding protein, such that translation of the NOI is repressed or prevented in a viral vector production cell.

Codon Optimisation

The polynucleotides used herein (including the NOI and/or components of the vector production system) may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. Many viruses, including retroviruses, use a large number of rare codons and changing these to correspond to commonly used mammalian codons, increases expression of a gene of interest, e.g. a NOI or packaging components in mammalian production cells, can be achieved. Codon usage tables are known in the art for mammalian cells, as well as for a variety of other organisms.

Codon optimisation of viral vector components has a number of other advantages. By virtue of alterations in their sequences, the nucleotide sequences encoding the packaging components of the viral particles required for assembly of viral particles in the producer cells/packaging cells have RNA instability sequences (INS) eliminated from them. At the same time, the amino acid sequence coding sequence for the packaging components is retained so that the viral components encoded by the sequences remain the same, or at least sufficiently similar that the function of the packaging components is not compromised. In lentiviral vectors codon optimisation also overcomes the Rev/RRE requirement for export, rendering optimised sequences Rev-independent. Codon optimisation also reduces homologous recombination between different constructs within the vector system (for example between the regions of overlap in the gag-pol and env open reading frames). The overall effect of codon optimisation is therefore a notable increase in viral titre and improved safety.

In one embodiment only codons relating to INS are codon optimised. However, in a much more preferred and practical embodiment, the sequences are codon optimised in their entirety, with some exceptions, for example the sequence encompassing the frameshift site of gag-pol (see below).

The gag-pol gene of lentiviral vectors comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimised. Retaining this fragment will enable more efficient expression of the Gag-Pol proteins. For EIAV the beginning of the overlap has been taken to be nt 1262 (where nucleotide 1 is the A of the gag ATG) and the end of the overlap to be nt 1461. In order to ensure that the frameshift site and the gag-pol overlap are preserved, the wild type sequence has been retained from nt 1156 to 1465. Derivations from optimal codon usage may be made, for example, in order to accommodate convenient restriction sites, and conservative amino acid changes may be introduced into the Gag-Pol proteins.

In one example, codon optimisation is based on lightly expressed mammalian genes. The third and sometimes the second and third base may be changed.

Due to the degenerate nature of the genetic code, it will be appreciated that numerous gag-pol sequences can be achieved by a skilled worker. Also there are many retroviral variants described which can be used as a starting point for generating a codon-optimised gag-pol sequence. Lentiviral genomes can be quite variable. For example there are many quasi-species of HIV-1 which are still functional. This is also the case for EIAV. These variants may be used to enhance particular parts of the transduction process. Examples of HIV-1 variants may be found at the HIV Databases operated by Los Alamos National Security, LLC at http://hiv-web.lanl.gov. Details of EIAV clones may be found at the National Center for Biotechnology Information (NCBI) database located at http://www.ncbi.nlm.nih.gov. The strategy for codon-optimised gag-pol sequences can be used in relation to any retrovirus. This would apply to all lentiviruses, including EIAV, FIV, BIV, CAEV, VMR, SIV, HIV-1 and HIV-2. In addition this method could be used to increase expression of genes from HTLV-1, HTLV-2, HFV, HSRV and human endogenous retroviruses (HERV), MLV and other retroviruses.

Codon optimisation can render gag-pol expression Rev-independent. In order to enable the use of anti-rev or RRE factors in the lentiviral vector, however, it would be necessary to render the viral vector generation system totally Rev/RRE-independent. Thus, the genome also needs to be modified. This is achieved by optimising vector genome components. Advantageously, these modifications also lead to the production of a safer system absent of all additional proteins both in the producer and in the transduced cell.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, N Y (1994); and Hale and Marham, The Harper Collins Dictionary of Biology, Harper Perennial, N.Y. (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole.

Aspects of the invention are demonstrated by the following non-limiting examples.

### EXAMPLES

### Example 1: Preliminary Evaluation of Small Molecule Induction Agents for Enhancing Vector Production in HEK293T

The inventors conducted a preliminary investigation into the use of alternative small molecule induction agents to increase vector titres in transiently transfected adherent HEK293T cells. Standard procedure in transient processes is to induce vector production at 24 h post transfection with 10 mM sodium butyrate, an aliphatic HDAC inhibitor. A preliminary high-throughput molecule screen was used to identify the impact on titre of using alternative HDAC inhibitors (sodium valproate, valeric acid, SAHA and TSA), a HAT inhibitor (tannic acid), a cell differentiating agent (HMBA), PKC agonists (prostratin and PMA), and an antioxidant agent (N-Acetyl Cysteine).

Materials and Methods

Adherent Cell Culture, Transfection and 3^{rd} Generation, SIN-Lentiviral Vector Production

HEK293T cells were maintained in complete media (Dulbecco's Modified Eagle Medium (DMEM) (Sigma) supplemented with 10% heat-inactivated fetal bovine serum (FBS) (Gibco), 2 mM L-glutamine (Sigma) and 1% non-essential amino acids (NEAA) (Sigma)), at 37° C. in 5% CO₂.

HIV CMV-GFP vector was produced at 12-well plate scale under the following conditions: HEK293T cells were seeded in complete media and approximately 24 hours later the cells were transfected with Genome, Gag-Pol, Rev and VSV-G. Transfection was mediated by mixing DNA with Lipofectamine 2000CD in OptiPRO as per manufacturer's protocol (Life Technologies).

An automated liquid handler was used to prepare 1 mL of induction mixture by diluting stock reagents in complete media to the final concentrations listed in Table 1. Cells were induced approximately 24 hours after transfection by discarding media and replacing with 0.8 mL induction mixture. Vector supernatant was harvested 24 hours later and filtered using a MultiScreen-GV 0.22 μm 96-well filter plate (Millipore).

TABLE 1
Concentration of tested induction reagents added to 12-well plate.

| Sodium | Sodium | Valeric | | | Tannic | | | | |
|---|---|---|---|---|---|---|---|---|---|
| Butyrate | Valproate | acid | NAC | HMBA | acid | Prostrain | SAHA | TSA | PMA |
| (mM) | (mM) | (mM) | (mM) | (mM) | (mM) | (mM) | (mM) | (mM) | (mM) |
| 0 | 0 | 0 | 0 | 0 | 0 | 0 | 0 | 0 | 0 |
| 5 | 0 | 0 | 0 | 0 | 0 | 0 | 0 | 0 | 0 |
| 10 | 0 | 0 | 0 | 0 | 0 | 0 | 0 | 0 | 0 |
| 20 | 0 | 0 | 0 | 0 | 0 | 0 | 0 | 0 | 0 |
| 0 | 5 | 0 | 0 | 0 | 0 | 0 | 0 | 0 | 0 |
| 0 | 10 | 0 | 0 | 0 | 0 | 0 | 0 | 0 | 0 |
| 0 | 20 | 0 | 0 | 0 | 0 | 0 | 0 | 0 | 0 |
| 0 | 0 | 5 | 0 | 0 | 0 | 0 | 0 | 0 | 0 |
| 0 | 0 | 10 | 0 | 0 | 0 | 0 | 0 | 0 | 0 |
| 0 | 0 | 20 | 0 | 0 | 0 | 0 | 0 | 0 | 0 |
| 0 | 0 | 0 | 1 | 0 | 0 | 0 | 0 | 0 | 0 |
| 0 | 0 | 0 | 2 | 0 | 0 | 0 | 0 | 0 | 0 |
| 0 | 0 | 0 | 4 | 0 | 0 | 0 | 0 | 0 | 0 |
| 0 | 0 | 0 | 0 | 1 | 0 | 0 | 0 | 0 | 0 |
| 0 | 0 | 0 | 0 | 2 | 0 | 0 | 0 | 0 | 0 |
| 0 | 0 | 0 | 0 | 4 | 0 | 0 | 0 | 0 | 0 |
| 0 | 0 | 0 | 0 | 0 | 0.05 | 0 | 0 | 0 | 0 |
| 0 | 0 | 0 | 0 | 0 | 0.1 | 0 | 0 | 0 | 0 |
| 0 | 0 | 0 | 0 | 0 | 0.2 | 0 | 0 | 0 | 0 |
| 0 | 0 | 0 | 0 | 0 | 0 | 0.0005 | 0 | 0 | 0 |
| 0 | 0 | 0 | 0 | 0 | 0 | 0.001 | 0 | 0 | 0 |
| 0 | 0 | 0 | 0 | 0 | 0 | 0.002 | 0 | 0 | 0 |
| 0 | 0 | 0 | 0 | 0 | 0 | 0 | 0.00125 | 0 | 0 |
| 0 | 0 | 0 | 0 | 0 | 0 | 0 | 0.0025 | 0 | 0 |
| 0 | 0 | 0 | 0 | 0 | 0 | 0 | 0.005 | 0 | 0 |
| 0 | 0 | 0 | 0 | 0 | 0 | 0 | 0 | 0.001 | 0 |
| 0 | 0 | 0 | 0 | 0 | 0 | 0 | 0 | 0.002 | 0 |
| 0 | 0 | 0 | 0 | 0 | 0 | 0 | 0 | 0.004 | 0 |
| 0 | 0 | 0 | 0 | 0 | 0 | 0 | 0 | 0 | 0.00000125 |
| 0 | 0 | 0 | 0 | 0 | 0 | 0 | 0 | 0 | 0.0000025 |
| 0 | 0 | 0 | 0 | 0 | 0 | 0 | 0 | 0 | 0.000005 |
| 10 | 0 | 0 | 0 | 0 | 0 | 0.001 | 0 | 0 | 0 |
| 0 | 0 | 10 | 0 | 2 | 0 | 0 | 0.0025 | 0.002 | 0.0000025 |
| 0 | 10 | 10 | 2 | 2 | 0.1 | 0 | 0 | 0.002 | 0 |
| 10 | 10 | 10 | 2 | 2 | 0.1 | 0.001 | 0.0025 | 0.002 | 0.0000025 |
| 0 | 10 | 0 | 0 | 2 | 0.1 | 0.001 | 0.0025 | 0.002 | 0 |
| 10 | 10 | 0 | 2 | 2 | 0 | 0.001 | 0 | 0 | 0 |
| 0 | 0 | 0 | 0 | 2 | 0.1 | 0 | 0 | 0 | 0 |
| 0 | 10 | 0 | 2 | 2 | 0 | 0 | 0.0025 | 0 | 0.0000025 |
| 0 | 0 | 10 | 2 | 0 | 0 | 0 | 0 | 0 | 0 |
| 5 | 5 | 5 | 1 | 1 | 0.05 | 0.0005 | 0.00125 | 0.001 | 0.00000125 |
| 0 | 10 | 0 | 0 | 0 | 0 | 0 | 0 | 0.002 | 0 |
| 10 | 10 | 10 | 0 | 0 | 0.1 | 0.001 | 0 | 0 | 0 |
| 10 | 0 | 0 | 0 | 0 | 0 | 0 | 0 | 0 | 0.0000025 |
| 10 | 0 | 0 | 2 | 0 | 0.1 | 0.001 | 0 | 0.002 | 0 |
| 0 | 0 | 10 | 2 | 2 | 0.1 | 0.001 | 0.0025 | 0 | 0 |
| 10 | 0 | 10 | 2 | 0 | 0 | 0.001 | 0.0025 | 0 | 0.0000025 |
| 0 | 10 | 10 | 0 | 0 | 0.1 | 0 | 0.0025 | 0 | 0.0000025 |
| 10 | 0 | 10 | 2 | 2 | 0.1 | 0 | 0 | 0 | 0.0000025 |
| 10 | 0 | 10 | 0 | 0 | 0.1 | 0 | 0.0025 | 0.002 | 0 |
| 10 | 10 | 0 | 2 | 0 | 0.1 | 0 | 0.0025 | 0 | 0 |
| 10 | 0 | 0 | 0 | 2 | 0.1 | 0.001 | 0.0025 | 0 | 0.0000025 |
| 0 | 10 | 10 | 0 | 2 | 0 | 0.001 | 0 | 0 | 0.0000025 |
| 10 | 0 | 0 | 2 | 2 | 0 | 0 | 0.0025 | 0.002 | 0 |
| 0 | 0 | 10 | 0 | 0 | 0.1 | 0.001 | 0 | 0.002 | 0.0000025 |
| 0 | 0 | 0 | 2 | 2 | 0 | 0.001 | 0 | 0.002 | 0.0000025 |
| 0 | 0 | 0 | 0 | 0 | 0 | 0.001 | 0.0025 | 0 | 0 |
| 10 | 10 | 10 | 0 | 2 | 0 | 0 | 0.0025 | 0 | 0 |
| 0 | 10 | 0 | 2 | 0 | 0.1 | 0.001 | 0 | 0 | 0.0000025 |
| 10 | 10 | 0 | 0 | 0 | 0 | 0.001 | 0.0025 | 0.002 | 0.0000025 |
| 0 | 0 | 0 | 2 | 0 | 0.1 | 0 | 0.0025 | 0.002 | 0.0000025 |
| 0 | 10 | 10 | 2 | 0 | 0 | 0.001 | 0.0025 | 0.002 | 0 |
| 10 | 10 | 10 | 2 | 0 | 0 | 0 | 0 | 0.002 | 0.0000025 |
| 10 | 0 | 10 | 0 | 2 | 0 | 0.001 | 0 | 0.002 | 0 |
| 10 | 10 | 0 | 0 | 2 | 0.1 | 0 | 0 | 0.002 | 0.0000025 |
| 0 | 0 | 0 | 0 | 0 | 0 | 0 | 0 | 0 | 0 |

Lentiviral Vector Titration Assay

For lentiviral vector titration by GFP marker-containing cassette, HEK293T cells were seeded in complete media. Wells were transduced approximately 24 hours after seeding with 270 μL vector diluted in complete media+8 μg/mL polybrene and wells were topped up with 530 μL complete media between 3-6 hours after transduction. The transduced cells were incubated for 3 days at 37° C. in 5% CO₂. Cells were detached using TrypLE and resuspended in complete media for flow cytometry. Percent GFP expression was measured using Live/Singlet/GFP⁺ gating. Titres were calculated based on percent GFP⁺ cells, a cell count at transduction of 1×10⁵, the vector dilution factor and volume vector at transduction, using the equation below.

$$Poisson Corrected Titre ( \frac{T U}{mL} ) = \frac{- ln ( 1 - ( \frac{\% {GFP}^{+}}{100} ) ) \cdot cell count at transduction \cdot dilution factor}{volume of vector at transduction ( mL )}$$

Results

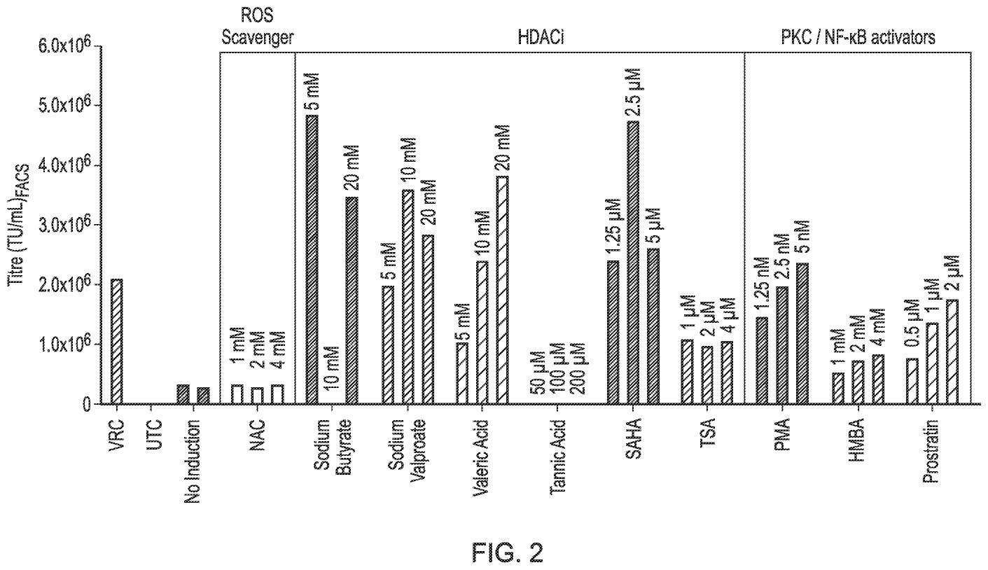

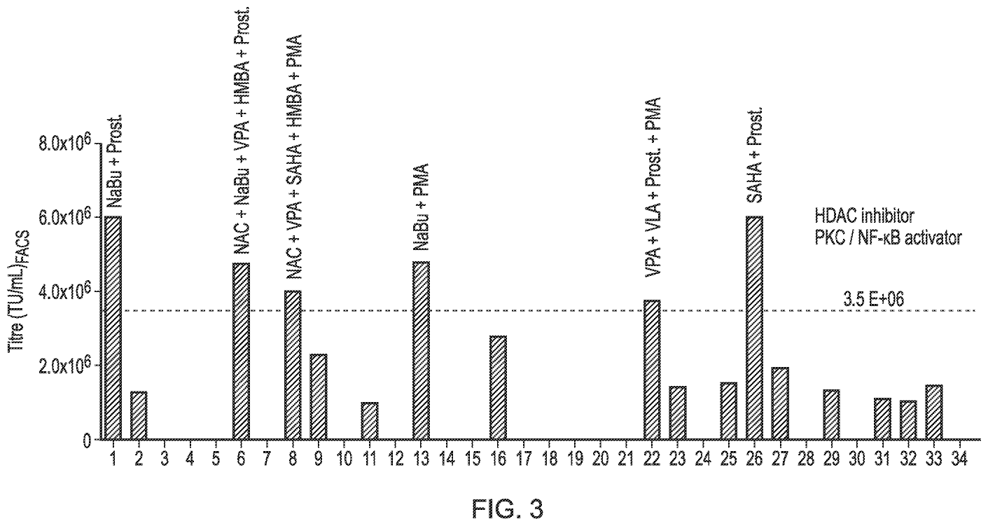

These results indicate that many of the tested HDAC inhibitors have induction effects in HEK293T, with optimum vector production at concentrations of 5 mM sodium butyrate, 10 mM sodium valproate, 20 mM valeric acid, and 2.5 μM SAHA (FIG. 2). TSA failed to improve vector titres in HEK293T cells above the level of 20 mM sodium butyrate. The antioxidant, NAC, had no positive impact on titre on its own, with basal vector production remaining the same in the presence of 1-4 mM NAC as with the no induction controls. Tannic acid had considerable negative effects on vector production, resulting in no measurable vector production. As a separate class of compounds, the transcription activators individually showed vector induction potential, with highest vector induction resulting from the PKC activators, PMA and prostratin. The randomised combination screen (FIG. 3) indicates that highest titres were achieved where HDAC inhibitors were combined with transcriptional activators, consistent with reports concerning increased virus production when HDAC inhibitors are combined with latency reversing agents in latent-HIV therapy (Reuse et al., 2009).

Discussion

The inventors have demonstrated that the combined use of HDAC inhibitors with PKC activators stimulates the greatest increase in vector titre.

### Example 2: Evaluation of Small Molecule Induction Agents for Enhancing Vector Production in HEK293T

The inventors further investigated the use of alternative small molecule induction agents to increase vector titres in transiently transfected HEK293T cells. Standard procedure in transient processes is to induce vector production at 24 h post transfection with 10 mM sodium butyrate, an aliphatic HDAC inhibitor. The use of high-throughput screening methods to investigate the use of two alternative aliphatic compounds (sodium valproate and valeric acid) and a hydroxamic acid compound (SAHA) as HDAC inhibitors is reported. In addition, the inventors investigated the effect of combining HDAC inhibitors with transcriptional activators, HMBA (a cell differentiating agent) and prostratin and PMA (PKC agonists) to increase titres.

Materials and Methods

Experiment 1

Adherent Cell Culture, Transfection and 3^{rd} Generation, SIN-Lentiviral Vector Production

HEK293T cells were maintained in complete media (Dulbecco's Modified Eagle Medium (DMEM) (Sigma) supplemented with 10% heat-inactivated fetal bovine serum (FBS) (Gibco), 2 mM L-glutamine (Sigma) and 1% non-essential amino acids (NEAA) (Sigma)), at 37° C. in 5% CO₂.

HIV CMV-GFP vector was produced at 12-well plate scale under the following conditions: HEK293T cells were seeded in 1 mL complete media and approximately 24 hours later the cells were transfected with Genome, Gag-Pol, Rev and VSV-G. Transfection was mediated by mixing DNA with Lipofectamine 2000CD in OptiPRO as per manufacturer's protocol (Life Technologies).

An automated liquid handler was used to prepare 1.2 mL of induction mixture by diluting stock reagents in complete media to the final concentrations listed in Table 2. Cells were induced approximately 24 hours after transfection by discarding media and replacing with 1 mL induction mixture. Vector supernatant was harvested approximately 24 hours later and filtered using a MultiScreen-GV 0.22 μm 96-well filter plate (Millipore).

TABLE 2
Concentration of tested induction reagents added to 12-well plate.

| | | Sodium | | | | | |
|---|---|---|---|---|---|---|---|
| | Sodium | Val- | Valeric | | | Pro- | |
| Con- | Butyrate | proate | acid | SAHA | HMBA | strain | PMA |
| dition | (mM) | (mM) | (mM) | (mM) | (mM) | (mM) | (mM) |
| 1 | 0 | 0 | 0 | 0 | 0 | 0 | 0 |
| 2 | 0 | 0 | 0 | 0 | 0 | 0 | 0 |
| 3 | 5 | 0 | 0 | 0 | 0 | 0 | 0 |
| 4 | 5 | 0 | 0 | 0 | 0 | 0 | 0 |
| 5 | 10 | 0 | 0 | 0 | 0 | 0 | 0 |
| 6 | 10 | 0 | 0 | 0 | 0 | 0 | 0 |
| 7 | 20 | 0 | 0 | 0 | 0 | 0 | 0 |
| 8 | 20 | 0 | 0 | 0 | 0 | 0 | 0 |
| 9 | 30 | 0 | 0 | 0 | 0 | 0 | 0 |
| 10 | 30 | 0 | 0 | 0 | 0 | 0 | 0 |
| 11 | 0 | 5 | 0 | 0 | 0 | 0 | 0 |
| 12 | 0 | 5 | 0 | 0 | 0 | 0 | 0 |
| 13 | 0 | 10 | 0 | 0 | 0 | 0 | 0 |
| 14 | 0 | 10 | 0 | 0 | 0 | 0 | 0 |
| 15 | 0 | 20 | 0 | 0 | 0 | 0 | 0 |
| 16 | 0 | 20 | 0 | 0 | 0 | 0 | 0 |
| 17 | 0 | 30 | 0 | 0 | 0 | 0 | 0 |
| 18 | 0 | 30 | 0 | 0 | 0 | 0 | 0 |
| 19 | 0 | 0 | 5 | 0 | 0 | 0 | 0 |
| 20 | 0 | 0 | 5 | 0 | 0 | 0 | 0 |
| 21 | 0 | 0 | 10 | 0 | 0 | 0 | 0 |
| 22 | 0 | 0 | 10 | 0 | 0 | 0 | 0 |
| 23 | 0 | 0 | 20 | 0 | 0 | 0 | 0 |
| 24 | 0 | 0 | 20 | 0 | 0 | 0 | 0 |
| 25 | 0 | 0 | 30 | 0 | 0 | 0 | 0 |
| 26 | 0 | 0 | 30 | 0 | 0 | 0 | 0 |
| 27 | 0 | 0 | 0 | 0.001 | 0 | 0 | 0 |
| 28 | 0 | 0 | 0 | 0.001 | 0 | 0 | 0 |
| 29 | 0 | 0 | 0 | 0.002 | 0 | 0 | 0 |
| 30 | 0 | 0 | 0 | 0.002 | 0 | 0 | 0 |
| 31 | 0 | 0 | 0 | 0.004 | 0 | 0 | 0 |
| 32 | 0 | 0 | 0 | 0.004 | 0 | 0 | 0 |
| 33 | 0 | 0 | 0 | 0.008 | 0 | 0 | 0 |
| 34 | 0 | 0 | 0 | 0.008 | 0 | 0 | 0 |
| 35 | 0 | 0 | 0 | 0.016 | 0 | 0 | 0 |
| 36 | 0 | 0 | 0 | 0.016 | 0 | 0 | 0 |
| 37 | 0 | 0 | 0 | 0 | 1 | 0 | 0 |
| 38 | 0 | 0 | 0 | 0 | 1 | 0 | 0 |
| 39 | 0 | 0 | 0 | 0 | 2 | 0 | 0 |
| 40 | 0 | 0 | 0 | 0 | 2 | 0 | 0 |
| 41 | 0 | 0 | 0 | 0 | 4 | 0 | 0 |
| 42 | 0 | 0 | 0 | 0 | 4 | 0 | 0 |
| 43 | 0 | 0 | 0 | 0 | 8 | 0 | 0 |
| 44 | 0 | 0 | 0 | 0 | 8 | 0 | 0 |
| 45 | 0 | 0 | 0 | 0 | 16 | 0 | 0 |
| 46 | 0 | 0 | 0 | 0 | 16 | 0 | 0 |
| 47 | 0 | 0 | 0 | 0 | 32 | 0 | 0 |
| 48 | 0 | 0 | 0 | 0 | 32 | 0 | 0 |
| 49 | 0 | 0 | 0 | 0 | 0 | 0.0005 | 0 |
| 50 | 0 | 0 | 0 | 0 | 0 | 0.0005 | 0 |
| 51 | 0 | 0 | 0 | 0 | 0 | 0.001 | 0 |
| 52 | 0 | 0 | 0 | 0 | 0 | 0.001 | 0 |
| 53 | 0 | 0 | 0 | 0 | 0 | 0.002 | 0 |
| 54 | 0 | 0 | 0 | 0 | 0 | 0.002 | 0 |
| 55 | 0 | 0 | 0 | 0 | 0 | 0.004 | 0 |
| 56 | 0 | 0 | 0 | 0 | 0 | 0.004 | 0 |
| 57 | 0 | 0 | 0 | 0 | 0 | 0.008 | 0 |
| 58 | 0 | 0 | 0 | 0 | 0 | 0.008 | 0 |
| 59 | 0 | 0 | 0 | 0 | 0 | 0.016 | 0 |
| 60 | 0 | 0 | 0 | 0 | 0 | 0.016 | 0 |
| 61 | 0 | 0 | 0 | 0 | 0 | 0 | 0.000001 |
| 62 | 0 | 0 | 0 | 0 | 0 | 0 | 0.000001 |
| 63 | 0 | 0 | 0 | 0 | 0 | 0 | 0.000002 |
| 64 | 0 | 0 | 0 | 0 | 0 | 0 | 0.000002 |
| 65 | 0 | 0 | 0 | 0 | 0 | 0 | 0.000004 |
| 66 | 0 | 0 | 0 | 0 | 0 | 0 | 0.000004 |
| 67 | 0 | 0 | 0 | 0 | 0 | 0 | 0.000008 |
| 68 | 0 | 0 | 0 | 0 | 0 | 0 | 0.000008 |
| 69 | 0 | 0 | 0 | 0 | 0 | 0 | 0.000016 |
| 70 | 0 | 0 | 0 | 0 | 0 | 0 | 0.000016 |
| 71 | 0 | 0 | 0 | 0 | 0 | 0 | 0.000032 |
| 72 | 0 | 0 | 0 | 0 | 0 | 0 | 0.000032 |
| 73 | 10 | 0 | 0 | 0 | 4 | 0 | 0 |
| 74 | 10 | 0 | 0 | 0 | 0 | 0.002 | 0 |
| 75 | 10 | 0 | 0 | 0 | 0 | 0 | 0.000004 |
| 76 | 0 | 10 | 0 | 0 | 4 | 0 | 0 |
| 77 | 0 | 10 | 0 | 0 | 0 | 0.002 | 0 |
| 78 | 0 | 10 | 0 | 0 | 0 | 0 | 0.000004 |
| 79 | 0 | 0 | 10 | 0 | 4 | 0 | 0 |
| 80 | 0 | 0 | 10 | 0 | 0 | 0.002 | 0 |
| 81 | 0 | 0 | 10 | 0 | 0 | 0 | 0.000004 |
| 82 | 0 | 0 | 0 | 0.002 | 4 | 0 | 0 |
| 83 | 0 | 0 | 0 | 0.002 | 0 | 0.002 | 0 |
| 84 | 0 | 0 | 0 | 0.002 | 0 | 0 | 0.000004 |

Lentiviral Vector Titration Assay

For lentiviral vector titration by GFP marker-containing cassette, HEK293T cells were seeded in complete media. Wells were transduced approximately 24 hours after seeding with 265 μL vector diluted in complete media+8 μg/mL polybrene and wells were topped up with 530 μL complete media between 3-6 hours after transduction. The transduced cells were incubated for 3 days at 37° C. in 5% CO₂. Cells were detached using TrypLE (Gibco) and resuspended in complete media for flow cytometry. Percent GFP expression was measured using Live/Singlet/GFP⁺ gating. Titres were calculated based on percent GFP⁺ cells, a cell count at transduction of 8.46×10⁴, the vector dilution factor and volume vector at transduction, using the equation below.

$$Poisson Corrected Titre ( \frac{T U}{mL} ) = \frac{- ln ( 1 - ( \frac{\% {GFP}^{+}}{100} ) ) \cdot cell count at transduction \cdot dilution factor}{volume of vector at transduction ( mL )}$$

Experiment 2

Adherent Cell Culture, Transfection and 3^{rd} Generation, SIN-Lentiviral Vector Production

HEK293T cells were maintained in complete media (Dulbecco's Modified Eagle Medium (DMEM) (Sigma) supplemented with 10% heat-inactivated fetal bovine serum (FBS) (Gibco), 2 mM L-glutamine (Sigma) and 1% non-essential amino acids (NEAA) (Sigma)), at 37° C. in 5% CO₂.

HIV CMV-GFP vector was produced at 12-well plate scale under the following conditions: HEK293T cells were seeded in 1 mL complete media and approximately 24 hours later the cells were transfected with Genome, Gag-Pol, Rev and VSV-G. Transfection was mediated by mixing DNA with Lipofectamine 2000CD in OptiPRO as per manufacturer's protocol (Life Technologies).

JMP was used to create a 3×3×2 full factorial DOE to screen sodium butyrate, prostratin and HMBA, and a 2×2×2 full factorial to screen alternate HDAC inhibitors, prostratin and HMBA. An automated liquid handler was used to prepare 1.2 mL of induction mixture by diluting stock reagents in complete media to the final concentrations listed in Table 3. Cells were induced approximately 24 hours after transfection by discarding media and replacing with 1 mL induction mixture. Vector supernatant was harvested 24 hours later and filtered using a MultiScreen-GV 0.22 μm 96-well filter plate (Millipore).

TABLE 3
Concentration of tested induction reagents added to 12-well plate.

| | Sodium | Sodium | Valeric | | | Pro- |
|---|---|---|---|---|---|---|
| Con- | Butyrate | Valproate | Acid | SAHA | HMBA | stratin |
| dition | (mM) | (mM) | (mM) | (mM) | (mM) | (mM) |
| 1 | 5 | 0 | 0 | 0 | 8 | 0.01 |
| 2 | 6.25 | 0 | 0 | 0 | 4 | 0.0055 |
| 3 | 10 | 0 | 0 | 0 | 0 | 0.0055 |
| 4 | 10 | 0 | 0 | 0 | 0 | 0.01 |
| 5 | 2.5 | 0 | 0 | 0 | 8 | 0.0055 |
| 6 | 6.25 | 0 | 0 | 0 | 4 | 0.0055 |
| 7 | 2.5 | 0 | 0 | 0 | 8 | 0.001 |
| 8 | 2.5 | 0 | 0 | 0 | 0 | 0.001 |
| 9 | 2.5 | 0 | 0 | 0 | 0 | 0.01 |
| 10 | 10 | 0 | 0 | 0 | 0 | 0.001 |
| 11 | 5 | 0 | 0 | 0 | 0 | 0.01 |
| 12 | 5 | 0 | 0 | 0 | 0 | 0.0055 |
| 13 | 2.5 | 0 | 0 | 0 | 0 | 0.0055 |
| 14 | 2.5 | 0 | 0 | 0 | 8 | 0.01 |
| 15 | 2.5 | 0 | 0 | 0 | 8 | 0.01 |
| 16 | 10 | 0 | 0 | 0 | 0 | 0.01 |
| 17 | 5 | 0 | 0 | 0 | 8 | 0.01 |
| 18 | 5 | 0 | 0 | 0 | 0 | 0.0055 |
| 19 | 5 | 0 | 0 | 0 | 8 | 0.0055 |
| 20 | 10 | 0 | 0 | 0 | 8 | 0.001 |
| 21 | 5 | 0 | 0 | 0 | 8 | 0.001 |
| 22 | 5 | 0 | 0 | 0 | 0 | 0.01 |
| 23 | 10 | 0 | 0 | 0 | 8 | 0.001 |
| 24 | 10 | 0 | 0 | 0 | 8 | 0.0055 |
| 25 | 5 | 0 | 0 | 0 | 8 | 0.001 |
| 26 | 2.5 | 0 | 0 | 0 | 0 | 0.01 |
| 27 | 10 | 0 | 0 | 0 | 8 | 0.0055 |
| 28 | 2.5 | 0 | 0 | 0 | 0 | 0.001 |
| 29 | 5 | 0 | 0 | 0 | 8 | 0.0055 |
| 30 | 10 | 0 | 0 | 0 | 8 | 0.01 |
| 31 | 5 | 0 | 0 | 0 | 0 | 0.001 |
| 32 | 2.5 | 0 | 0 | 0 | 0 | 0.0055 |
| 33 | 10 | 0 | 0 | 0 | 0 | 0.0055 |
| 34 | 2.5 | 0 | 0 | 0 | 8 | 0.001 |
| 35 | 2.5 | 0 | 0 | 0 | 8 | 0.0055 |
| 36 | 10 | 0 | 0 | 0 | 8 | 0.01 |
| 37 | 5 | 0 | 0 | 0 | 0 | 0.001 |
| 38 | 10 | 0 | 0 | 0 | 0 | 0.001 |
| 39 | 0 | 5 | 0 | 0 | 8 | 0.01 |
| 40 | 0 | 7.5 | 0 | 0 | 4 | 0.0055 |
| 41 | 0 | 5 | 0 | 0 | 0 | 0.01 |
| 42 | 0 | 10 | 0 | 0 | 8 | 0.01 |
| 43 | 0 | 5 | 0 | 0 | 8 | 0.01 |
| 44 | 0 | 10 | 0 | 0 | 0 | 0.001 |
| 45 | 0 | 5 | 0 | 0 | 0 | 0.001 |
| 46 | 0 | 10 | 0 | 0 | 8 | 0.001 |
| 47 | 0 | 10 | 0 | 0 | 8 | 0.01 |
| 48 | 0 | 10 | 0 | 0 | 0 | 0.01 |
| 49 | 0 | 5 | 0 | 0 | 8 | 0.001 |
| 50 | 0 | 5 | 0 | 0 | 8 | 0.001 |
| 51 | 0 | 5 | 0 | 0 | 0 | 0.001 |
| 52 | 0 | 10 | 0 | 0 | 0 | 0.001 |
| 53 | 0 | 10 | 0 | 0 | 8 | 0.001 |
| 54 | 0 | 10 | 0 | 0 | 0 | 0.01 |
| 55 | 0 | 5 | 0 | 0 | 0 | 0.01 |
| 56 | 0 | 7.5 | 0 | 0 | 4 | 0.0055 |
| 57 | 0 | 0 | 5 | 0 | 8 | 0.01 |
| 58 | 0 | 0 | 10 | 0 | 0 | 0.001 |
| 59 | 0 | 0 | 5 | 0 | 0 | 0.001 |
| 60 | 0 | 0 | 7.5 | 0 | 4 | 0.0055 |
| 61 | 0 | 0 | 10 | 0 | 8 | 0.001 |
| 62 | 0 | 0 | 5 | 0 | 8 | 0.001 |
| 63 | 0 | 0 | 10 | 0 | 8 | 0.001 |
| 64 | 0 | 0 | 10 | 0 | 8 | 0.01 |
| 65 | 0 | 0 | 7.5 | 0 | 4 | 0.0055 |
| 66 | 0 | 0 | 5 | 0 | 8 | 0.01 |
| 67 | 0 | 0 | 5 | 0 | 0 | 0.001 |
| 68 | 0 | 0 | 10 | 0 | 0 | 0.01 |
| 69 | 0 | 0 | 5 | 0 | 0 | 0.01 |
| 70 | 0 | 0 | 10 | 0 | 0 | 0.001 |
| 71 | 0 | 0 | 5 | 0 | 0 | 0.01 |
| 72 | 0 | 0 | 10 | 0 | 8 | 0.01 |
| 73 | 0 | 0 | 5 | 0 | 8 | 0.001 |
| 74 | 0 | 0 | 10 | 0 | 0 | 0.01 |
| 75 | 0 | 0 | 0 | 0.001 | 8 | 0.001 |
| 76 | 0 | 0 | 0 | 0.0005 | 0 | 0.01 |
| 77 | 0 | 0 | 0 | 0.0005 | 8 | 0.01 |
| 78 | 0 | 0 | 0 | 0.001 | 8 | 0.01 |
| 79 | 0 | 0 | 0 | 0.001 | 8 | 0.001 |
| 80 | 0 | 0 | 0 | 0.001 | 0 | 0.01 |
| 81 | 0 | 0 | 0 | 0.001 | 8 | 0.01 |
| 82 | 0 | 0 | 0 | 0.001 | 0 | 0.01 |
| 83 | 0 | 0 | 0 | 0.0005 | 8 | 0.001 |
| 84 | 0 | 0 | 0 | 0.0005 | 0 | 0.01 |
| 85 | 0 | 0 | 0 | 0.00075 | 4 | 0.0055 |
| 86 | 0 | 0 | 0 | 0.0005 | 0 | 0.001 |
| 87 | 0 | 0 | 0 | 0.00075 | 4 | 0.0055 |
| 88 | 0 | 0 | 0 | 0.001 | 0 | 0.001 |
| 89 | 0 | 0 | 0 | 0.001 | 0 | 0.001 |
| 90 | 0 | 0 | 0 | 0.0005 | 8 | 0.001 |
| 91 | 0 | 0 | 0 | 0.0005 | 8 | 0.01 |
| 92 | 0 | 0 | 0 | 0.0005 | 0 | 0.001 |
| 93 | 0 | 0 | 0 | 0 | 0 | 0 |
| 94 | 0 | 0 | 0 | 0 | 0 | 0 |
| 95 | 10 | 0 | 0 | 0 | 0 | 0 |
| 96 | 10 | 0 | 0 | 0 | 0 | 0 |

Lentiviral Vector Titration Assay

For lentiviral vector titration by GFP marker-containing cassette, HEK293T cells were seeded in complete media. Wells were transduced approximately 24 hours after seeding with 265 μL vector diluted in complete media+8 μg/mL polybrene and wells were topped up with 530 μL complete media between 3-6 hours after transduction. The transduced cells were incubated for 3 days at 37° C. in 5% CO₂. Cells were detached using TrypLE (Gibco) and resuspended in complete media for flow cytometry. Percent GFP expression was measured using Live/Singlet/GFP⁺ gating. Titres were calculated based on percent GFP⁺ cells, a cell count at transduction of 7.98×10⁴, the vector dilution factor and volume vector at transduction, using the equation below.

$$Poisson Corrected Titre ( \frac{T U}{mL} ) = \frac{- ln ( 1 - ( \frac{\% {GFP}^{+}}{100} ) ) \cdot cell count at transduction \cdot dilution factor}{volume of vector at transduction ( mL )}$$

Results

Experiment 1

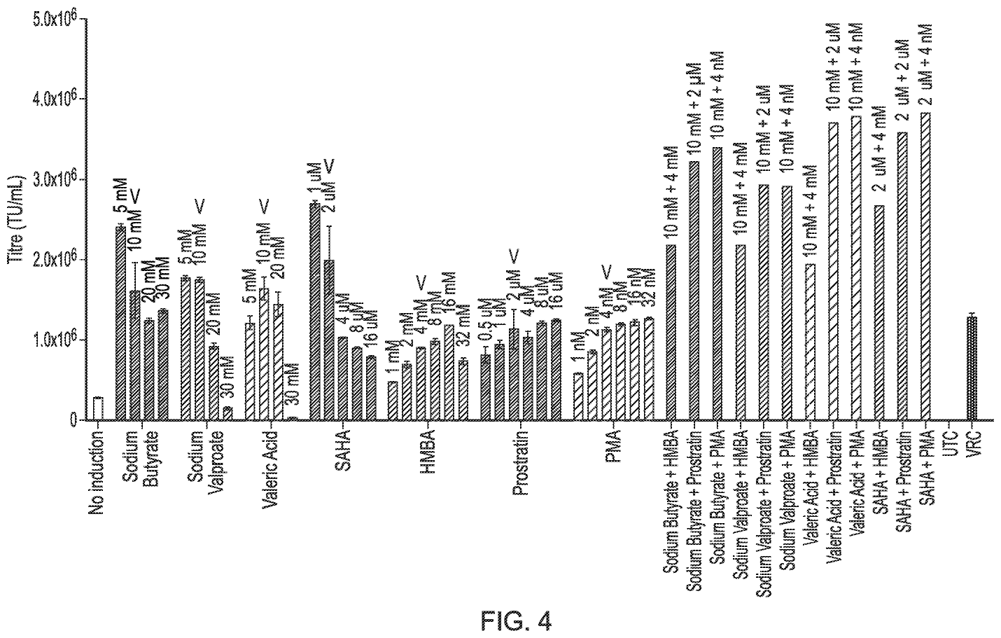

The results (see FIG. 4) indicate that the tested HDAC inhibitors have similar induction effects, with optimum concentrations of 10 mM sodium valproate, 10 mM valeric acid, and 1 μM SAHA. The transcriptional activators all showed good inducing effects on cells, with optimum concentrations of 16 mM HMBA, 16 μM prostratin and 32 nM PMA producing approximately 4-fold higher titres than the no-induction control. Notably, the greatest increase in vector titre was induced by combinations of HDAC inhibitors with the tested PKC agonists (prostratin and PMA), both of which induced similar increases of between 1.6- to 2.0-fold above the corresponding HDAC concentration. In contrast to prostratin, the combination of HMBA with HDAC inhibitors showed no or only marginal improvements in titres compared to those induced by the equivalent concentrations of HDAC inhibitor alone.

The MFI from GFP FACS was not observed to change in the concentration ranges of 0.5 to 8 μM prostratin. However, the MFI increased by 26% at 16 μM prostratin, indicating that a residual concentration of 0.4 μM prostratin after vector dilution (40-fold) was sufficient to impact transgene synthesis in transduced cells.

Experiment 2

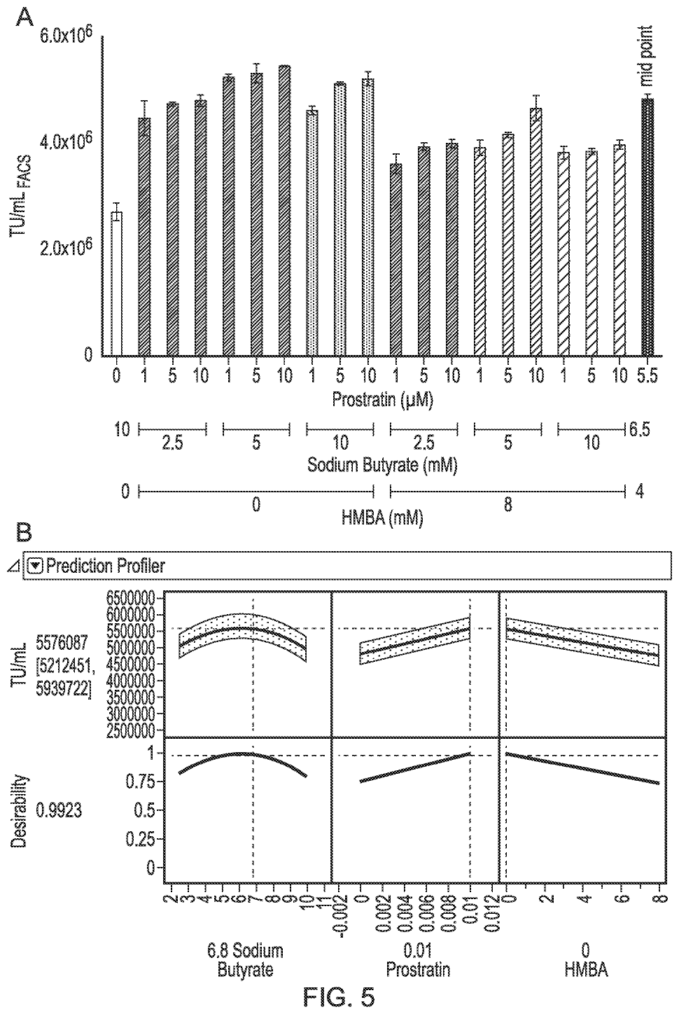

The aim of Experiment 2 was to investigate whether HMBA has any positive effect on titre in combination with both HDAC inhibitor and prostratin. To investigate interactions between varying concentrations of sodium butyrate, prostratin and HMBA, a 3×3×2 full-factorial DOE was performed (FIG. 5). The presence of prostratin in combination with 10 mM sodium butyrate showed good enhancement of vector production, increasing titres by 92% at a concentration of 10 μM prostratin. The prediction profiler indicated that presence of higher concentrations of prostratin gave higher desirability scores compared to 1 or 5 μM prostratin (FIG. 5B). Despite exhibiting inductive effects on its own in Experiment 1, titres in the presence of 8 mM HMBA in combination with prostratin and sodium butyrate showed a notable decrease compared to conditions where HMBA was excluded.

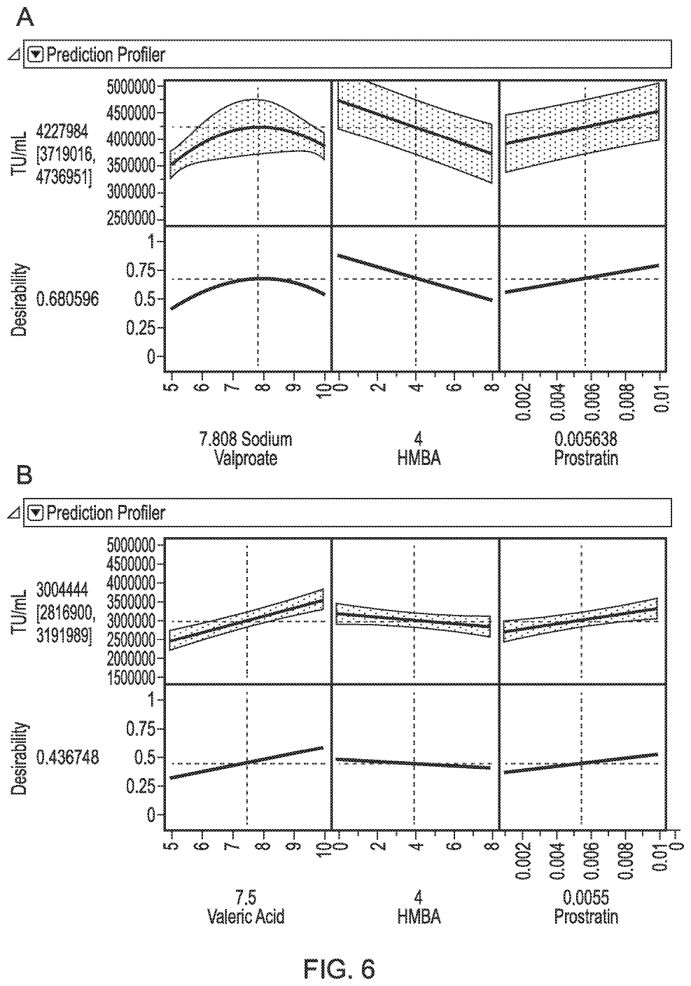

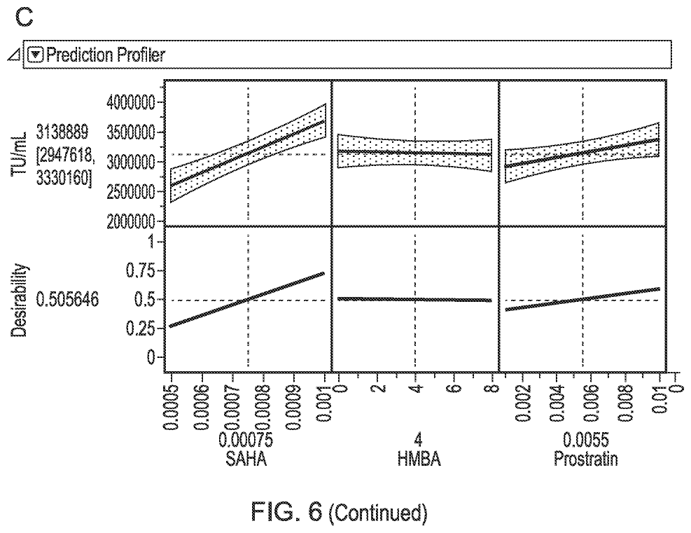

Similarly, HMBA showed negative effects on titre in combination with the alternate HDAC inhibitors: sodium valproate, valeric acid and SAHA, suggesting that HMBA does not provide any enhancing benefit as an additive alongside HDAC inhibitors and prostratin. Nevertheless, the positive enhancement of induction from increasing prostratin from 1 to 10 μM was common throughout all the tested HDAC inhibitors, supporting the effectiveness of PKC agonists as a potential enhancer of vector induction (FIG. 6).

Discussion

These experiments conclude that the combined use of HDAC inhibitors with PKC activators results in an increase in vector titre between 1.6- to 2.0-fold above what would be expected from induction with corresponding HDAC inhibitors on their own. Consistent with their structural and functional likeness, PMA showed similar enhancing effects as prostratin. However, the use of prostratin (or other similar analogue) may be favoured over PMA for inducing the production of medicinal vector due to its non-tumorigenic properties. It is noted that HMBA did not produce any positive effects on titre in combination with other small molecule induction agents.

### Example 3: Evaluation of Small Molecule Induction Agents for Enhancing Vector Production in HEK1.65S

The inventors investigated the use of alternative small molecule induction agents to increase vector titres in the transiently transfected suspension-adapted HEK293T (HEK1.65s) cells. Standard procedure in transient processes is to induce vector production at 24 h post transfection with 10 mM sodium butyrate, an aliphatic HDAC inhibitor. The use of high-throughput screening methods to investigate the use of two aliphatic compounds (sodium valproate and valeric acid) and a hydroxamic acid (SAHA) as alternative HDAC inhibitors is reported. In addition, the inventors investigated the effect of combining these HDAC inhibitors with the non-tumour-promoting PKC activator, prostratin, to increase titres of GFP HIV vector.

Materials and Methods

Experiment 1

Suspension Cell Culture, Transfection and 3^{rd} Generation, SIN-Lentiviral Vector Production

HEK1.65s cells were grown in serum-free Freestyle (FS) media+0.1% cholesterol lipid concentrate (CLC) (Gibco) at 37° C. in 5% CO₂ in a shaking incubator.

HIV CMV-GFP vector was produced in 24-well low attachment plates under the following conditions: HEK1.65s cells were seeded in 1 mL serum-free media and approximately 24 hours later the cells were transfected with Genome, Gag-Pol, Rev and VSV-G. Transfection was mediated by mixing DNA with Lipofectamine 2000CD in serum-free media as per manufacturer's protocol (Life Technologies). Cells were incubated at 37° C. in 5% CO₂ in a shaking incubator throughout vector production.

JMP was used to prepare a 2×2 full factorial condition matrix for each HDACi with 2× centre points and 2× replicates of each condition. An automated liquid handler was used to prepare a 96-well plate with 250 μL of 12× concentrated induction mixtures. 100 μL of 12× concentrated induction mixture was pipetted into the corresponding wells of each 24-well plate to give final concentrations listed in Table 4. Vector supernatant was harvested 2 days later and filtered using a MultiScreen-GV 0.22 μm 96-well filter plate (Millipore).

TABLE 4
Final concentration of tested induction reagents
upon addition to 24-well plate.

| | Sodium | Sodium | Valeric | | |
|---|---|---|---|---|---|
| | Butyrate | Valproate | Acid | SAHA | Prostratin |
| Condition | (mM) | (mM) | (mM) | (mM) | (mM) |
| 1 | 3 | 0 | 0 | 0 | 0 |
| 2 | 3 | 0 | 0 | 0 | 0 |
| 3 | 3 | 0 | 0 | 0 | 0.008 |
| 4 | 3 | 0 | 0 | 0 | 0.008 |
| 5 | 6.5 | 0 | 0 | 0 | 0.004 |
| 6 | 6.5 | 0 | 0 | 0 | 0.004 |
| 7 | 6.5 | 0 | 0 | 0 | 0.004 |
| 8 | 6.5 | 0 | 0 | 0 | 0.004 |
| 9 | 10 | 0 | 0 | 0 | 0 |
| 10 | 10 | 0 | 0 | 0 | 0 |
| 11 | 10 | 0 | 0 | 0 | 0.008 |
| 12 | 10 | 0 | 0 | 0 | 0.008 |
| 13 | 0 | 3 | 0 | 0 | 0 |
| 14 | 0 | 3 | 0 | 0 | 0 |
| 15 | 0 | 3 | 0 | 0 | 0.008 |
| 16 | 0 | 3 | 0 | 0 | 0.008 |
| 17 | 0 | 6.5 | 0 | 0 | 0.004 |
| 18 | 0 | 6.5 | 0 | 0 | 0.004 |
| 19 | 0 | 6.5 | 0 | 0 | 0.004 |
| 20 | 0 | 6.5 | 0 | 0 | 0.004 |
| 21 | 0 | 10 | 0 | 0 | 0 |
| 22 | 0 | 10 | 0 | 0 | 0 |
| 23 | 0 | 10 | 0 | 0 | 0.008 |
| 24 | 0 | 10 | 0 | 0 | 0.008 |
| 25 | 0 | 0 | 3 | 0 | 0 |
| 26 | 0 | 0 | 3 | 0 | 0 |
| 27 | 0 | 0 | 3 | 0 | 0.008 |
| 28 | 0 | 0 | 3 | 0 | 0.008 |
| 29 | 0 | 0 | 6.5 | 0 | 0.004 |
| 30 | 0 | 0 | 6.5 | 0 | 0.004 |
| 31 | 0 | 0 | 6.5 | 0 | 0.004 |
| 32 | 0 | 0 | 6.5 | 0 | 0.004 |
| 33 | 0 | 0 | 10 | 0 | 0 |
| 34 | 0 | 0 | 10 | 0 | 0 |
| 35 | 0 | 0 | 10 | 0 | 0.008 |
| 36 | 0 | 0 | 10 | 0 | 0.008 |
| 37 | 0 | 0 | 0 | 0.001 | 0 |
| 38 | 0 | 0 | 0 | 0.001 | 0 |
| 39 | 0 | 0 | 0 | 0.001 | 0.008 |
| 40 | 0 | 0 | 0 | 0.001 | 0.008 |
| 41 | 0 | 0 | 0 | 0.0015 | 0.004 |
| 42 | 0 | 0 | 0 | 0.0015 | 0.004 |
| 43 | 0 | 0 | 0 | 0.0015 | 0.004 |
| 44 | 0 | 0 | 0 | 0.0015 | 0.004 |
| 45 | 0 | 0 | 0 | 0.002 | 0 |
| 46 | 0 | 0 | 0 | 0.002 | 0 |
| 47 | 0 | 0 | 0 | 0.002 | 0.008 |
| 48 | 0 | 0 | 0 | 0.002 | 0.008 |

Lentiviral Vector Titration Assay

For lentiviral vector titration by GFP marker-containing cassette, HEK293T cells were seeded in complete media. Wells were transduced approximately 24 hours after seeding with 160 μL vector diluted in complete media+8 μg/mL polybrene and wells were topped up with 320 μL complete media between 3-6 hours after transduction. The transduced cells were incubated for 3 days at 37° C. in 5% CO₂. Cells were detached using TrypLE (Gibco) and resuspended in complete media for flow cytometry. Percent GFP expression was measured using Live/Singlet/GFP⁺ gating. Titres were calculated based on percent GFP⁺ cells, a cell count at transduction of 6.7×10⁴, the vector dilution factor and volume vector at transduction, using the equation below.

$$Poisson Corrected Titre ( \frac{T U}{mL} ) = \frac{- ln ( 1 - ( \frac{\% {GFP}^{+}}{100} ) ) \cdot cell count at transduction \cdot dilution factor}{volume of vector at transduction ( mL )}$$

Experiment 2

Suspension Cell Culture, Transfection and 3^{rd} Generation, SIN-Lentiviral Vector Production

HEK1.65s cells were grown in serum-free Freestyle (FS) media+0.1% cholesterol lipid concentrate (CLC) (Gibco) at 37° C. in 5% CO₂ in a shaking incubator.

HIV CMV-GFP vector was produced in 24-well low attachment plates under the following conditions: HEK1.65s cells were seeded in 1 mL serum-free media and approximately 24 hours later the cells were transfected with Genome, Gag-Pol, Rev and VSV-G. Transfection was mediated by mixing DNA with Lipofectamine 2000CD in serum-free media as per manufacturer's protocol (Life Technologies). Cells were incubated at 37° C. in 5% CO₂ in a shaking incubator throughout vector production.

JMP was used to prepare a 4×5 full factorial condition matrix with 2× centre points and 2× replicates of each condition. An automated liquid handler was used to prepare a 96-well plate with 250 μL of 12× concentrated induction mixtures. 100 μL of 12× concentrated induction mixture was pipetted into the corresponding wells of each 24-well plate to give final concentrations listed in Table 5. Vector supernatant was harvested 2 days later and filtered using a MultiScreen-GV 0.22 μm 96-well filter plate (Millipore).

TABLE 5
Final concentration of tested induction reagents
upon addition to 24-well plate.

| | Sodium | Prostratin |
|---|---|---|
| Condition | Butyrate (mM) | (mM) |
| 1 | 0 | 0 |
| 2 | 0 | 0 |
| 3 | 0 | 0.002 |
| 4 | 0 | 0.002 |
| 5 | 0 | 0.004 |
| 6 | 0 | 0.004 |
| 7 | 0 | 0.008 |
| 8 | 0 | 0.008 |
| 9 | 0 | 0.016 |
| 10 | 0 | 0.016 |
| 11 | 3 | 0 |
| 12 | 3 | 0 |
| 13 | 3 | 0.002 |
| 14 | 3 | 0.002 |
| 15 | 3 | 0.004 |
| 16 | 3 | 0.004 |
| 17 | 3 | 0.008 |
| 18 | 3 | 0.008 |
| 19 | 3 | 0.016 |
| 20 | 3 | 0.016 |
| 21 | 5 | 0.008 |
| 22 | 5 | 0.008 |
| 23 | 5 | 0.008 |
| 24 | 5 | 0.008 |
| 25 | 6.5 | 0 |
| 26 | 6.5 | 0 |
| 27 | 6.5 | 0.002 |
| 28 | 6.5 | 0.002 |
| 29 | 6.5 | 0.004 |
| 30 | 6.5 | 0.004 |
| 31 | 6.5 | 0.008 |
| 32 | 6.5 | 0.008 |
| 33 | 6.5 | 0.016 |
| 34 | 6.5 | 0.016 |
| 35 | 10 | 0 |
| 36 | 10 | 0 |
| 37 | 10 | 0.002 |
| 38 | 10 | 0.002 |
| 39 | 10 | 0.004 |
| 40 | 10 | 0.004 |
| 41 | 10 | 0.008 |
| 42 | 10 | 0.008 |
| 43 | 10 | 0.016 |
| 44 | 10 | 0.016 |
| 45 | 0 | 0.032 |
| 46 | 0 | 0.032 |
| 47 | 0 | 0 |
| 48 | 0 | 0 |

Lentiviral Vector Titration Assay

For lentiviral vector titration by GFP marker-containing cassette, HEK293T cells were seeded in complete media. Wells were transduced approximately 24 hours after seeding with 270 μL vector diluted in complete media+8 μg/mL polybrene and wells were topped up with 540 μL complete media between 3-6 hours after transduction. The transduced cells were incubated for 3 days at 37° C. in 5% CO₂. Cells were detached using TrypLE (Gibco) and resuspended in complete media for flow cytometry. Percent GFP expression was measured using Live/Singlet/GFP⁺ gating. Titres were calculated based on percent GFP⁺ cells, a cell count at transduction of 1.18×10⁵, the vector dilution factor and volume vector at transduction, using the equation below.

$$Poisson Corrected Titre ( \frac{T U}{mL} ) = \frac{- ln ( 1 - ( \frac{\% {GFP}^{+}}{100} ) ) \cdot cell count at transduction \cdot dilution factor}{volume of vector at transduction ( mL )}$$

Results

Experiment 1

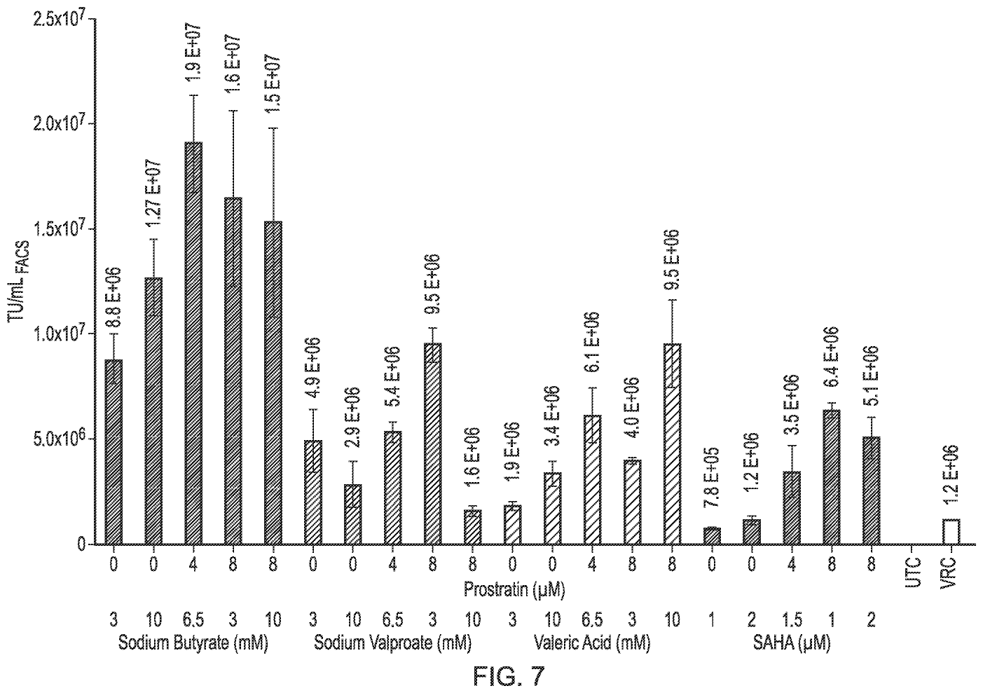

Of the different HDACi's tested on HEK1.65s cells, sodium butyrate induced the highest LV titres (FIG. 7). Surprisingly, SAHA exhibited the lowest inducing effect and yielded the lowest LV titres, despite previous data indicating SAHA stimulated comparable LV yields to sodium butyrate in transiently transfected HEK293T cells. In nearly all cases, the addition of prostratin alongside HDACi resulted in increased titres, supporting the prospective application of PKC agonists in HEK1.65s as inducing agents. Given the results of Experiment 1, Experiment 2 was conducted to establish a more detailed model of the combined effects of the best performing HDAC inhibitor (sodium butyrate) alongside prostratin to determine optimum concentrations of each induction agent.

Experiment 2

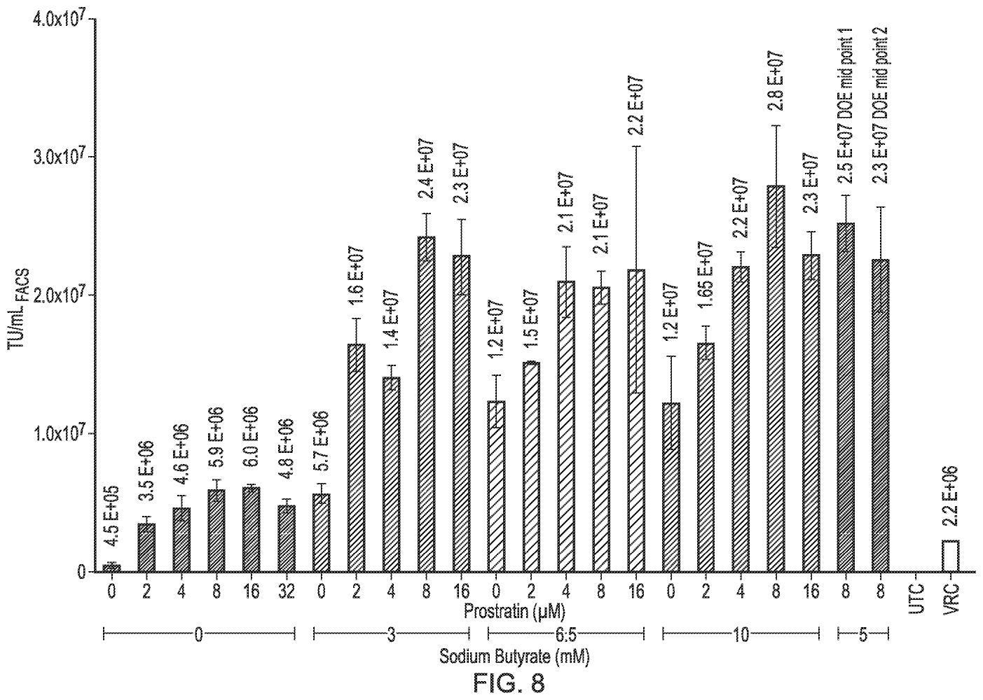

The results of Experiment 2 show that the prostratin has a marked positive effect on titre in combination with sodium butyrate in transiently transfected HEK1.65s suspension cells (FIG. 8). From the data shown herein, it is observed that concentrations of prostratin as low as 2-4 μM cause an increase in titre, with further increases observed at concentrations between 8-16 μM. Interestingly, these data also support previous findings that prostratin alone promotes increased vector titres, in this instance, increasing titres ˜13 fold at concentrations between 8-16 μM compared to the no induction control, and demonstrating approximately half the inducing effect of optimum sodium butyrate concentrations.

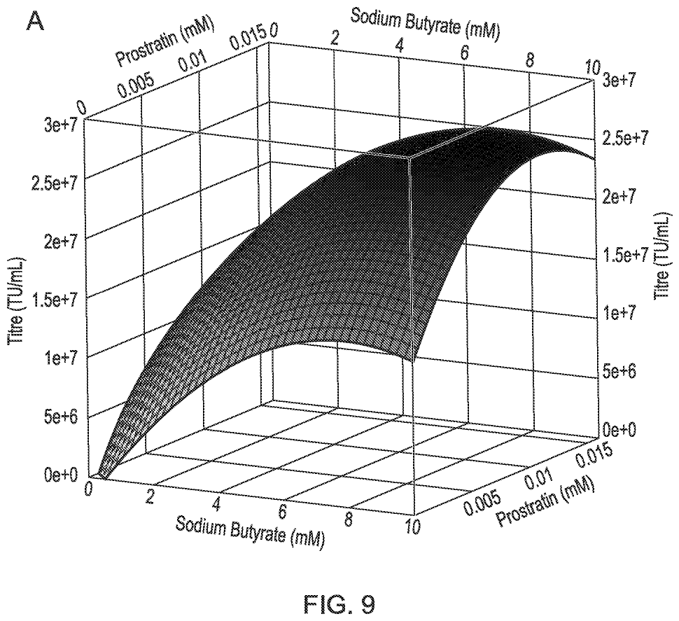

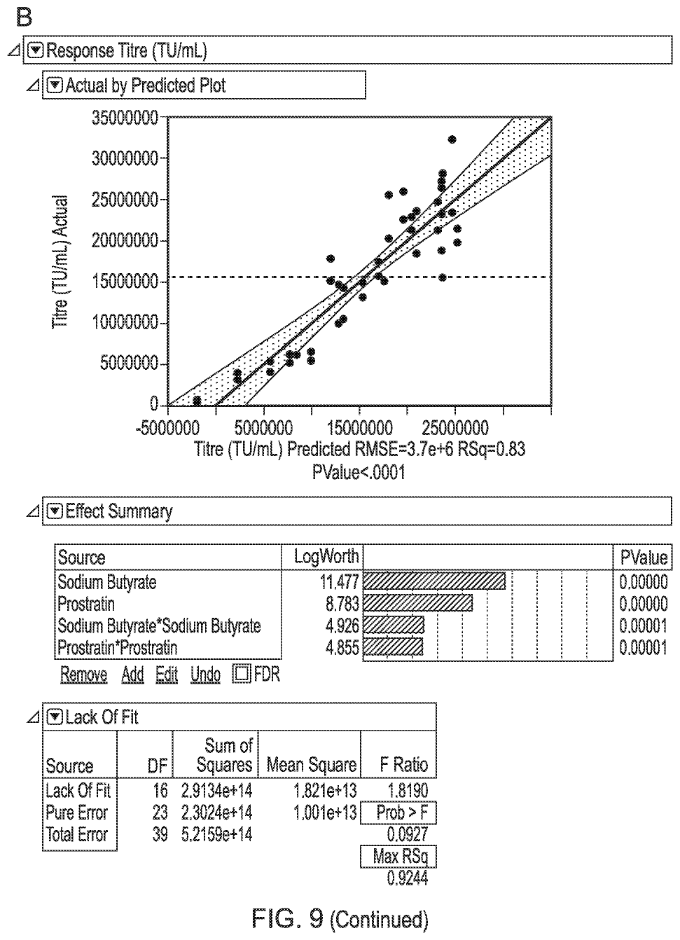

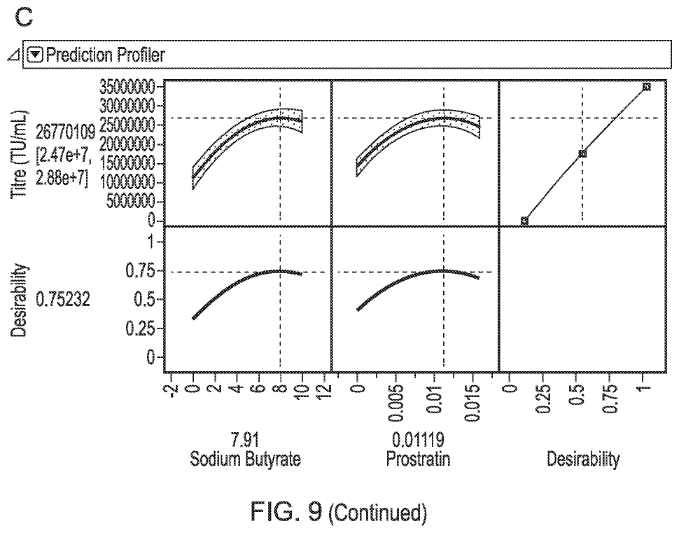

Resulting titres were returned into JMP DOE software to produce a model of the interactions of sodium butyrate and prostratin on LV titre (FIG. 9). The ‘actual by predicted’ plot (FIG. 9B) shows a strong fit between the DOE model and variation of collected data due to random effects. Log Worth values of sodium butyrate (11.5), prostratin (8.8), sodium butyrate*sodium butyrate (4.9), and prostratin*prostratin (4.9) were all in excess of 2, indicating that the significance of each effect greatly exceeds a threshold p value of 0.01. The prediction profiler (FIG. 9C) shows that the optimum predicted concentrations of the combined induction agents are: 8 mM sodium butyrate and 11 μM prostratin. Under these optimal conditions, LV titre is increased by 1.93 fold above optimum concentrations of sodium butyrate (8 mM) alone.

Cell viability measurements indicate that prostratin treated cells in the concentration range 2-32 μM did not present any further loss in cell viability compared to the 4% loss observed in sodium butyrate treated cells (Table 6). These results indicate that prostratin has little cytotoxic effect on cells over a vector production period of 48 hours.

TABLE 6
Cell viability measurements

| Condition | Description | Cell Viability (%) | Δ |
|---|---|---|---|
| 1 & 2 | No Induction Control | 78.8 ± 0.4 | N/A |
| 35 | 10 mM Sodium Butyrate | 74.8 | −4 |
| 3 | 2 μM Prostratin | 79.4 | +0.6 |
| 9 | 16 μM Prostratin | 82.2 | +3.4 |
| 45 | 32 μM Prostratin | 79.8 | +1 |

Discussion

The results show that prostratin is an effective enhancer of LV titres in transiently transfected HEK1.65s. Inducing transfected cells with 8-16 μM prostratin alone results in >10-fold increase in vector titre above the no-induction control. Furthermore, at the optimum concentrations established in this DOE model, 11 μM prostratin with 8 mM sodium butyrate increases LV titres nearly 2-fold above induction with optimum sodium butyrate conditions. In addition to its inducing effects, no decrease in cell viability was observed as a consequence of cellular exposure to prostratin. Although some inducing effect was observed using alternative aliphatic HDAC inhibitors (sodium valproate at 3 mM and valeric acid at 10 mM), the hydroxamic acid HDAC inhibitor, SAHA, presented the weakest inducing effect, and none of the alternate HDAC inhibitors increased titres above sodium butyrate at the concentrations tested in this experiment. This demonstrates that prostratin is a candidate small molecule for induction either on its own, or in combination with sodium butyrate in standard transient vector production methods.

### Example 4: Prostratin as a Small Molecule Enhancer of Vector Induction: 40 mL Shake Flask Study

Prostratin is a small molecule, non-tumour promoting modulator of protein kinase C (PKC) that has demonstrated promising therapeutic properties for the treatment of cancer (Alotaibi et al., 2018) and Alzheimer's disease (Hongpaisan & Alkon, 2007). In Example 3, the inventors demonstrated that prostratin is effective at increasing GFP-HIV LV titres in combination with HDAC inhibitors by ≥2-fold at 24-well plate scale in the transient LV production process using HEK1.65s. The inventors performed the following scale-up experiment to determine whether the prostratin-enhanced productivity of HEK cells translates to shake flask volumes (40 mL).

Materials and Methods

Suspension Cell Culture, Transfection and 3^{rd} Generation, SIN-Lentiviral Vector Production

HEK1.65s cells were grown in serum-free Freestyle (FS) media+0.1% cholesterol lipid concentrate (CLC) (Gibco) at 37° C. in 5% CO₂ in a shaking incubator.

HIV CMV-GFP vector was produced in six 125 mL Erlenmeyer shake flasks under the following conditions: HEK1.65s cells were seeded in 40 mL serum-free media and approximately 24 hours later the cells were transfected with Genome, Gag-Pol, Rev and VSV-G. Transfection was mediated by mixing DNA with Lipofectamine 2000CD in serum-free media as per manufacturer's protocol (Life Technologies). Cells were incubated at 37° C. in 5% CO₂ in a shaking incubator throughout the course of vector production.

Vector production in shake flasks 1-6 was induced using a final concentration of 8 mM sodium butyrate. At the same time as sodium butyrate induction, DMSO was added to flasks 3 & 4 to give a final concentration of 0.2% (v/v) DMSO, and prostratin (dissolved in DMSO) was added to shake flasks 5 & 6 to give a final concentration of 11 μM prostratin and 0.2% (v/v) DMSO (Table 7). Vector supernatant was harvested approximately 24 hours after induction and 0.45 μm filtered.

TABLE 7
Induction compositions of shake flasks.

| Shake Flask | NaBut (mM) | DMSO % (v/v) | Prostratin (μM) |
|---|---|---|---|
| 1 | 8 | N/A | N/A |
| 2 | 8 | N/A | N/A |
| 3 | 8 | 0.2 | N/A |
| 4 | 8 | 0.2 | N/A |
| 5 | 8 | 0.2 | 11 |
| 6 | 8 | 0.2 | 11 |

Lentiviral Vector Titration Assay

For lentiviral vector titration by FACS, HEK293T cells were seeded in complete media. Wells were transduced approximately 24 hours after seeding with 157 μL vector diluted in complete media+8 μg/mL polybrene and wells were topped up with 314 μL complete media between 3-6 hours after transduction. The transduced cells were incubated for 3 days at 37° C. in 5% CO₂. Cells were detached using TrypLE (Gibco) and resuspended in complete media for flow cytometry. Percent GFP expression was measured using Live/Singlet/GFP⁺ gating. Titres were calculated based on percent GFP⁺ cells, a cell count at transduction of 4.4×10⁴, the vector dilution factor and volume vector at transduction, using the equation below.

$$Poisson Corrected Titre ( \frac{T U}{mL} ) = \frac{- ln ( 1 - ( \frac{\% {GFP}^{+}}{100} ) ) \cdot cell count at transduction \cdot dilution factor}{volume of vector at transduction ( mL )}$$

For lentiviral vector titration by duplex QPCR integration assay, HEK293T cells were seeded in complete media. Wells were transduced approximately 24 hours after seeding with 500 μL vector diluted in complete media 8 μg/mL polybrene and wells were topped up with 1 mL complete media between 3-6 hours after transduction. Cultures were passaged for 10 days before host DNA was extracted from 1×10⁶ cell pellets. Duplex quantitative PCR was carried out using a FAM primer/probe set to the HIV packaging signal (ψ) and to RRPH1, and vector titres (TU/mL) calculated using the following factors: transduction volume, vector dilution, RRPH1-normalised HIV-1 ψ copies detected per reaction.

Results and Discussion

The translation of DOE-optimised induction conditions (8 mM sodium butyrate+11 μM prostratin: Example 3) to shake flask scale successfully demonstrated an increase in HIV-GFP titres. In the presence of prostratin, vector titres were 2.4- to 2.9-fold higher than titres achieved by inducing HEK1.65s cells with sodium butyrate alone (FIGS. 10A & B). The identical titres reported for the VRC and VRC+55 nM prostratin demonstrated that titres determined from the integration assay were not affected by residual concentrations of prostratin remaining after vector dilution (FIG. 10B).

### Example 5: Exemplary Modified U1 SNRNA Expression Constructs that May be Co-Expressed During Sin-Lentiviral Vector Production in the Context of the Invention

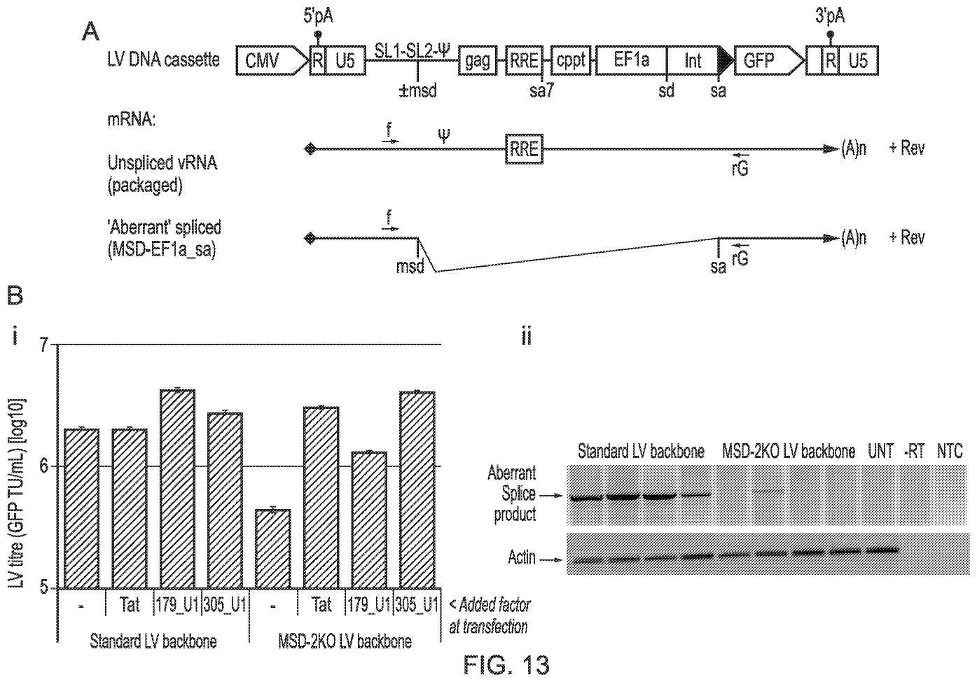

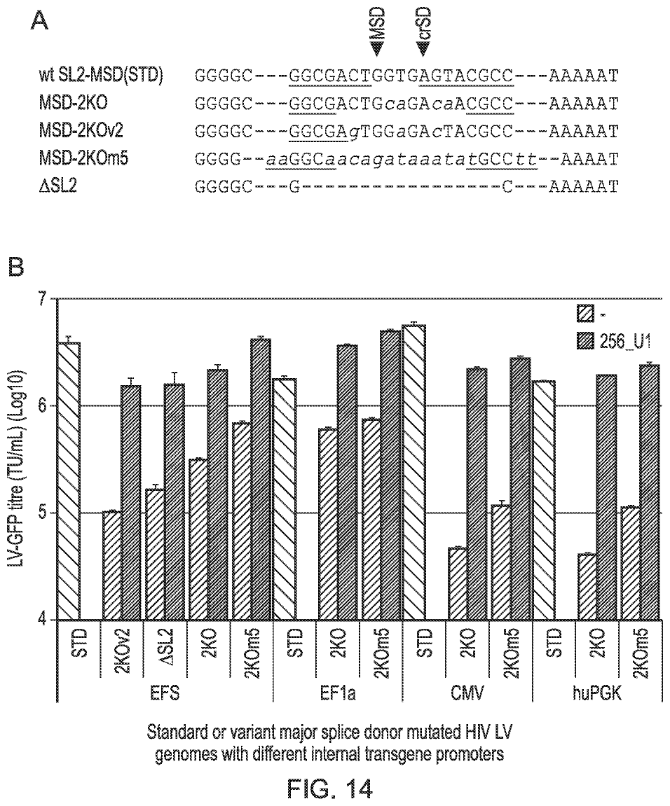

The inventors have shown previously that U1 snRNA can be modified, and co-expressed with lentiviral vectors (LVs) leading to enhanced production titres. An example of a modified U1 snRNA molecule is displayed in FIG. 11. Briefly, the native splice-donor site annealing sequence (nucleotides 1-11) may be replaced with a sequence that is complementary to a ‘target’ sequence within the 5′region of the lentiviral vector RNA (vRNA)— typically within the core packaging region—and expressed in parallel to the vRNA and other vector components, leading to an increase in vector titres. Without wishing to be bound by theory, it is hypothesised that modified U1 snRNAs bind to vRNA within the nucleus and ultimately stabilises/increases the steady state pool of vRNA available for packaging into virions. The inventors have shown previously that the major splice donor (MSD) region embedded within the packaging signal of lentiviral vector genome vRNA can be highly promiscuous, splicing to strong or cryptic splice acceptors within transgene sequences (even in the presence of rev) leading to a reduction in the amount of full length vRNA available for packaging (see FIG. 1). Ablation of this aberrant splicing activity is achieved by functional mutation or deletion of the MSD and a cryptic splice donor encoded a few nucleotides downstream (see FIG. 14A). This type of modification to LVs leads to a reduction in production titres; however, titres are boosted/recovered by the supply of the modified U1 snRNA during LV production, whilst also maintaining the block to aberrant splicing (see FIGS. 13 and 14).

The inventors wished to evaluate whether the use of Prostratin in the production of MSD-mutated LVs might lead to an enhancement in output titres, and whether both Prostratin and modified U1 snRNA might be applied together.

Suspension Cell Culture, Transfection and 3^{rd} Generation, SIN-Lentiviral Vector Production

HEK293T.1-65s suspension cells were grown in Freestyle+0.1% CLC (Gibco) at 37° C. in 5% CO₂, in a shaking incubator (25 mm orbit set at 190 RPM). HEK293Ts cells were seeded at 8×10⁵ cells per ml in serum-free media and were incubated at 37° C. in 5% CO₂, shaking, throughout vector production. Approximately 24 hours after seeding the cells were transfected using the following mass ratios of plasmids per effective final volume of culture at transfection: Genome, Gag-Pol, Rev, VSV-G, and between 0.01 to 0.2 μg/mL modified U1 snRNA plasmid when utilised.

Transfection was mediated by mixing DNA with Lipofectamine 2000CD in Opti-MEM as per manufacturer's protocol (Life Technologies). Sodium butyrate (Sigma) was added ˜18 hrs later to 10 mM final concentration, and optionally Prostratin was added along with sodium butyrate at a final concentration of 11 μM. Typically, vector supernatant was harvested 20-24 hours later, and then filtered (0.22 μm) and frozen at −20/−80° C. As a positive control for nuclease treatment, typically Benzonase® was added to the harvests at 5U/mL for 1 hour prior to filtration.

For evaluation of Prostratin and modified U1 snRNA, the standard SIN-LV genomes used were HIV-EFS-GFP or HIV-EF1a-GFP, and the MSD-mutated SIN-LV genomes were HIV-MSD2KOm5-EFS-GFP or MSD2KOm5-EF1a-GFP. The MSD2KOm5 modification is displayed in FIG. 14A. The modified U1 snRNA was 256U1, which targets the SL1 loop of the packaging signal (see SEQ ID No: 22); Table 8 shows other examples of targeting sequences of modified U1 snRNAs.

Lentiviral Vector Titration Assays

For lentiviral vector titration by GFP marker-containing cassette, HEK293T cells were seeded at 1.2×10⁴ cells/well in 96-well plates. GFP-encoding viral vectors were used to transduce the cells in complete media containing 8 mg/ml polybrene and 1×Penicillin Streptomycin for approximately 5-6 hours after which fresh media was added. The transduced cells were incubated for 2 days at 37° C. in 5% CO₂. Cultures were then prepared for flow cytometry using an Attune-NxT (Thermofisher). Percent GFP expression was measured and vector titres were calculated using a predicted cell count of 2×10⁴ cells at the time of transduction (base on typical growth rate), the dilution factor of the vector sample, the percentage positive GFP population and total volume at transduction.

Results

Surprisingly, it was found that the addition of Prostratin during SIN-LV production boosted titres of the MSD-mutated SIN-LV vectors (as well as standard SIN-LVs)—see FIG. 15. Moreover, when Prostratin was supplied together with 256U1 during production the output titres of the MSD-mutated SIN-LV vectors was increased higher than standard SIN-LV vectors in the absence of inducers. This data shows for the first time that a combination of chemical and polynucleotide-based inducer molecules can increase titres of both standard and MSD-mutated SIN-LVs.

The DNA-Based Expression Constructs for the Modified U1 snRNAs Comprise the Conserved Sequences in the Endogenous U1 snRNA Gene Driving RNA Transcription and Termination, Highlighted Below in the Non-Limiting Example of the 256U1 (Also Referred to as U1_256) snRNA:

(SEQ ID NO: 22)
TAAGGACCAGCTTCTTTGGGAGAGAACAGACGCAG
GGGCGGGAGGGAAAAAGGGAGAGGCAGACGTCACT
TCCCCTTGGCGGCTCTGGCAGCAGATTGGTCGGTT
GAGTGGCAGAAAGGCAGACGGGGACTGGGCAAGGC
ACTGTCGGTGACATCACGGACAGGGCGACTTCTAT
GTAGATGAGGCAGCGCAGAGGCTGCTGCTTCGCCA
CTTGCTGCTTCACCACGAAGGAGTTCCCGTGCCCT
GGGAGCGGGTTCAGGACCGCTGATCGGAAGTGAGA
ATCCCAGCTGTGTGTCAGGGCTGGAAAGGGCTCGG
GAGTGCGCGGGGCAAGTGACCGTGTGTGTAAAGAG
TGAGGCGTATGAGGCTGTGTCGGGGCAGAGGCCCA
AGATCTCatttgccgtgcgcgcttGCAGGGGAGAT
ACCATGATCACGAAGGTGGTTTTCCCAGGGCGAGG
CTTATCCATTGCACTCCGGATGTGCTGACCCCTGC
GATTTCCCCAAATGTGGGAAACTCGACTGCATAAT
TTGTGGTAGTGGGGGACTGCGTTCGCGCTTTCCCC
TGGTTTCAAAAGTAGACTGTACGCTAAGGGTCATA
TCTTTTTTTGTTTTGGTTTGTGTCTTGGTTGGCGT
CTTAAATGTTAA
Key:
Upper case only = U1 PoIII promoter (nt1-392);
lower case = retargeting region (nt393-409);
lowercase bold = retargeting sequence [in
this example targeting nt256-270 of wild
type HIV-1 packaging signal] (nt395-409);
upper case italics = main U1 snRNA sequence
[clover-leaf] (nt410-562);
upper case underlined = transcription
termination region (nt563-652)

A summary of the initial modified U1 snRNAs and controls used by the inventors is presented in the table below, indicating the new annealing sequence and the target site sequence (sequences are represented in the 5′ to 3′ direction).

TABLE 8
A list of sequences describing the target-annealing sequences (heterologous
sequence that is complementary to the target sequence) within test modified U1
snRNAs and control U1 snRNAs, and their target sequences used in the initial study.
Nucleotides are presented as DNA as they would be encoded within their respective
expression cassettes at the ‘retargeting region’. The (AT) motif was present in
all initial constructs, which forms the first two nucleotides of the U1 snRNA
molecule in each case. The target sequence numbers refer to targets in the
NL4-3 (GenBank:M19921.2) or HXB2 (GenBank: K03455.1) strains of HIV-1 where
denoted, since the lentiviral vector genome in this study contained a hybrid
packaging signal composed of these two highly conserved strains (packaging
sequence used in this study is most similar to the vector sequence in
GenBank: MH782475.1)

| Modified U1 | | U1 snRNA target- |
|---|---|---|
| snRNA* | HIV-1 target sequence [NL4-3]** | annealing sequence |
| U1_16 | 16-GACCAGATCTGAGCC-30 | (AT)GGCTCAGATCTGGTC |
| | (SEQ ID NO: 23) | (SEQ ID NO: 24) |
| U1_31 | 31-TGGGAGCTCTCTGGC-45 | (AT)GCCAGAGAGCTCCCA |
| | (SEQ ID NO: 25) | (SEQ ID NO: 26) |
| U1_76 | 76-TAAAGCTTGCCTTGA-90 | (AT)TCAAGGCAAGCTTTA |
| | (SEQ ID NO: 27) | (SEQ ID NO: 28) |
| U1_136 | 136-TAGAG ATCCCTCAG A-150 | (AT)TCTGAGGGATCTCTA |
| | (SEQ ID NO: 29) | (SEQ ID NO: 30) |
| U1_179 | 179-GCAGTGGCG-187 (SEQ ID | (AT)CGCCACTGC (SEQ ID |
| (9 nt) | NO: 31) | NO: 32) |
| U1_181 | 181-AGTGGCGCCCGAACA-195 | (AT)TGTTCGGGCGCCACT |
| | (SEQ ID NO: 33) | (SEQ ID NO: 34) |
| U1_196 | 196-GGGACTTGAAAGCGA-210 | (AT)TCGCTTTCAAGTCCC |
| | (SEQ ID NO: 35) | (SEQ ID NO: 36) |
| U1_211 | 211-AAGggAAaCCAGAGG-225 | (AT)CCTCTGGTTTCCCTT |
| | (SEQ ID NO: 37) | (SEQ ID NO: 38) |
| U1_226 | 226-AGcTCTCTCGACGCA-240 | (AT)TGCGTCGAGAGAGCT |
| | (SEQ ID NO:39) | (SEQ ID NO: 40) |
| U1_241 | 241-GGACTCGGCTTGCTG-255 | (AT)CAGCAAGCCGAGTCC |
| | (SEQ ID NO: 41) | (SEQ ID NO: 42) |
| U1_256 | 256-AAGCGCGCACGGCAA-270 | (A T) T TGCCGTGCGCGCTT |
| | (SEQ ID NO: 43) | (SEQ ID NO: 44) |
| U1_271 | 271-GAGGCGAGGGGCGGC-285 | (AT)GCCGCCCCTCGCCTC |
| | (SEQ ID NO: 45) | (SEQ ID NO: 46) |
| U1_286 | 286-GACTGGTGAGTACGC-300 | (AT)GCGTACTCACCAGTC |
| | (SEQ ID NO: 47) | (SEQ ID NO: 48) |
| U1_305 | 305-AATTTTGAC(TA)-313/5 | (AT)GTCAAAATT (SEQ ID |
| (9 nt) | (SEQ ID NO: 49) | NO: 50) |
| U1_305 | 305-AAT T T TGACTAGCGG-319 | (AT)CCGCTAGTCAAAATT |
| | (SEQ ID NO: 51) | (SEQ ID NO: 52) |
| U1_316 | 316-GCGGAGGCTAGAAGG-330 | (AT)CCTTCTAGCCTCCGC |
| | (SEQ ID NO: 53) | (SEQ ID NO: 54) |
| U1_331 | 331-AGAGAGATGGGTGCG-345 | (AT)CGCACCCATCTCTCT |
| | (SEQ ID NO: 55) | (SEQ ID NO: 56) |
| U1_346 | 346-AGAGCGTCgGTATTA-360 | (AT)TAATACTGACGCTCT |
| | (SEQ ID NO: 57) | (SEQ ID NO: 58) |
| U1_361 | 361-AGCGGGGGAGAATTA-375 | (AT)TAATTCTCCCCCGCT |
| | (SEQ ID NO: 59) | (SEQ ID NO: 60) |
| U1_376 | 376-GATCGCGATGGGAAA-390 | (AT)TTTCCCATCGCGATC |
| | (SEQ ID NO: 61) | (SEQ ID NO: 62) |
| U1_391 | 389-AAATTCGGTTAAGGC-403 | (AT)GCCTTAACCGAATTT |
| | (SEQ ID NO: 63) | (SEQ ID NO: 64) |
| U1_690 | 7159-GATCTTCAGACCTGG- | (AT)CCAGGTCTGAAGATC |
| | 7173 (SEQ ID NO: 65) | (SEQ ID NO: 66) |
| U1_1203 | 7672-TTACACAAGCTTAAT-7686 | (AT)ATTAAGCTTGTGTAA |
| | (SEQ ID NO: 67) | (SEQ ID NO: 68) |
| U11546 | 4375-TAGTAGACATAATAG-4389 | (AT)CTATTATGTCTACTA |
| | (SEQ ID NO: 69) | (SEQ ID NO: 70) |
| | Target | U1 snRNA target- |
| Control U1 | sequence | snRNA annealing sequence |
| U1_LacZ1 | 388-CTACAGGAA-396 (SEQ ID | (AT) T TCCTGTAG (SEQ ID |
| | NO: 71) | NO: 72) |
| U1_LacZ2 | 438-TCATCTGTG-446 (SEQ ID | (AT)CACAGATGA (SEQ ID |
| | NO: 73) | NO: 74) |

*numbering relative to vector genome RNA sequence
**lower case target sequence is for (HXB2), underlined target sequence is an AA > CGCG frameshift in the gag ORF (111 376)

### Example 6: Evaluation of the Individual and Combined Effects of Modified U1 SNRNA Expression and Prostratin Induction for the Production of Therapeutic Vector in Transiently Transfected HEK1.65s

Proceeding from previously observed increases in SIN-LV vector titre carrying GFP reporter transgene, the inventors further wished to investigate the individual and combined use of 256U1 and prostratin for enhancing the production of HIV vector comprising therapeutic transgenes (CAR #1, CAR #2 and CAR #2-T2A-GFP) in transiently transfected HEK1.65s cells. Titre increases were compared to the standard SIN-LV production procedure for the same transgenes in the absence of polynucleotide or small molecule inducing agents.

Materials and Methods

Suspension Cell Culture, Transfection and 3^{rd} Generation, SIN-Lentiviral Vector Production

HEK1.65s cells were grown in serum-free Freestyle (FS) media+0.1% cholesterol lipid concentrate (CLC) (Gibco) at 37° C. in 5% CO₂ in a shaking incubator.

HIV CAR #1, CAR #2 and CAR #2-T2A-GFP vector was produced in twelve 125 mL Erlenmeyer shake flasks under the following conditions: HEK1.65s cells were seeded in 20 mL serum-free media and approximately 24 hours later the cells were transfected with Genome, Gag-Pol, Rev and VSV-G. 256U1 plasmid was co-transfected in shake flasks 2, 4, 6, 8, 10 and 12 (Table 9). Transfection was mediated by mixing DNA with Lipofectamine 2000CD in serum-free media as per manufacturer's protocol (Life Technologies). Cells were incubated at 37° C. in 5% CO₂ in a shaking incubator throughout the course of vector production.

Vector production in shake flasks 1-12 was induced using a final concentration of 10 mM sodium butyrate approximately 24 hours after transfection. At the same time as sodium butyrate induction, prostratin (dissolved in DMSO) was added to shake flasks 3, 4, 7, 8, 11 and 12 to give a final concentration of 11 μM prostratin and 0.2% (v/v) DMSO (Table 9). Vector supernatant was harvested approximately 24 hours after induction and 0.45 μm filtered.

TABLE 9
Experimental conditions for investigating the impact of
modified U1 snRNA and prostratin on vector titre.

| Shake Flask | Genome | 256U1 (μg/mL) | Prostratin (μM) |
|---|---|---|---|
| 1 | CAR#1 | N/A | N/A |
| 2 | CAR#1 | 0.4 | N/A |
| 3 | CAR#1 | N/A | 11 |
| 4 | CAR#1 | 0.4 | 11 |
| 5 | CAR#2 | N/A | N/A |
| 6 | CAR#2 | 0.4 | N/A |
| 7 | CAR#2 | N/A | 11 |
| 8 | CAR#2 | 0.4 | 11 |
| 9 | CAR#2-T2A-GFP | N/A | N/A |
| 10 | CAR#2-T2A-GFP | 0.4 | N/A |
| 11 | CAR#2-T2A-GFP | N/A | 11 |
| 12 | CAR#2-T2A-GFP | 0.4 | 11 |

Lentiviral Vector Titration Assay

For lentiviral vector titration by FACS, HEK293T cells were seeded in complete media. Wells were transduced approximately 24 hours after seeding with 50 μL vector diluted in complete media+8 μg/mL polybrene and wells were topped up with 200 μL complete media between 3-6 hours after transduction. The transduced cells were incubated for 3 days at 37° C. in 5% CO₂. Cells were detached using TrypLE (Gibco), resuspended in complete media and washed with phosphate-buffered saline (PBS) prior to antibody staining for flow cytometry. For CAR #1 and CAR #2 vector, percent scFv (single-chain variable fragment) expression was measured using Live/Singlet/scFv⁺ gating, for CAR #2-T2A-GFP vector, percent scFv and GFP was measured using Live/Singlet/scFv⁺&GFP⁺ gating. Titres were calculated based on percent scFv⁺ or scFv⁺&GFP⁺ cells, a cell count at transduction of 1.65×10⁴, the vector dilution factor and volume vector at transduction, using the equation below.

$$Poisson Corrected Titre ( \frac{T U}{mL} ) = \frac{\begin{matrix}- ln ( 1 - ( \frac{\% {scFv}^{+} ( \& {GFP}^{+} )}{100} ) ) \cdot \\ cell count at transduction \cdot dilution factor\end{matrix}}{volume of vector at transduction ( mL )}$$

Results and Discussion

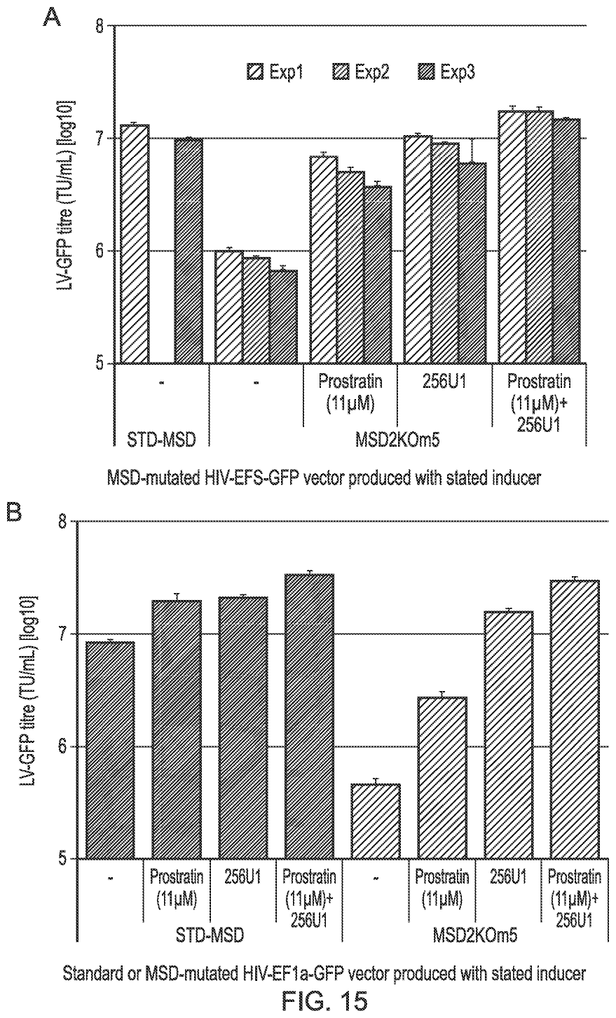

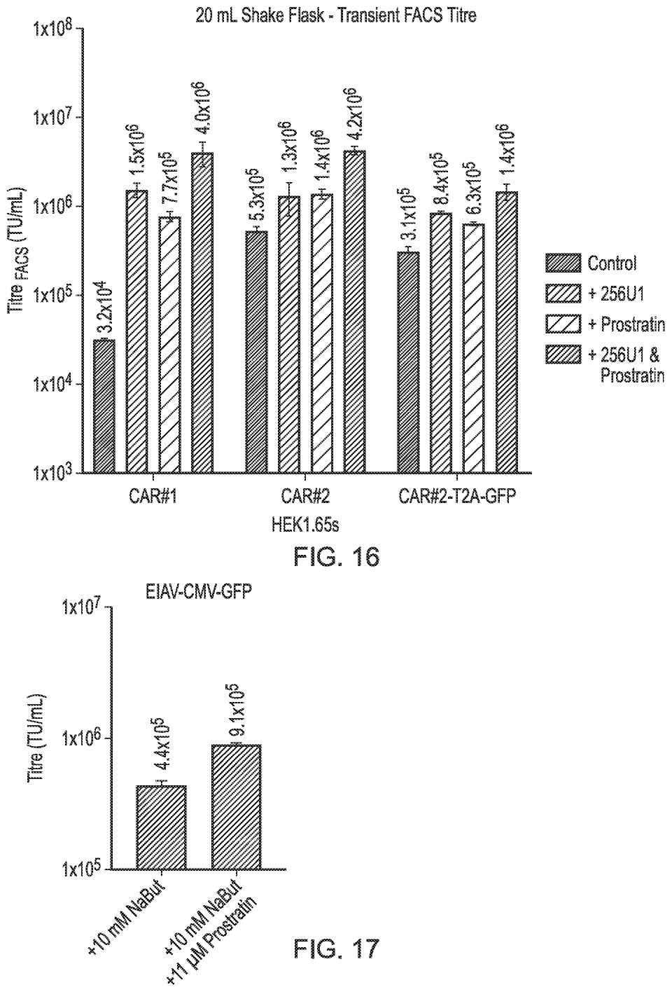

Titre increases for all three CAR vector products were observed when 256U1 snRNA was expressed by producing cells compared to the standard production procedure control flasks (46-fold for CAR #1, 2.5-fold for CAR #2, and 2.7-fold for CAR #2-T2A-GFP) (FIG. 16). Similarly, titre increases for the three vector products were also observed when prostratin was included with sodium butyrate during vector induction (24-fold for CAR #1, 2.6-fold for CAR #2, and 2-fold for CAR #2-T2A-GFP). Notably, the greatest increases in vector titres were achieved in shake flasks where 256U1 snRNA expression was combined with the addition of prostratin at induction (125-fold for CAR #1, 7.9-fold for CAR #2 and 4.5-fold for CAR #2-T2A-GFP). These results support the inventor's previous observations made in Example 5, and indicate for the first time in therapeutic vector production, that polynucleotide and small molecule inducing agents can be combined to increase vector titre within the transient SIN-LV production process.

### Example 7: Prostratin as a Small Molecule Enhancer of EIAV Vector Induction

Prostratin is a small molecule, non-tumour promoting modulator of protein kinase C (PKC) that has demonstrated promising therapeutic properties for the treatment of cancer (Alotaibi et al., 2018) and Alzheimer's disease (Hongpaisan & Alkon, 2007). In Example 4, the inventors demonstrated that prostratin is effective at increasing GFP-HIV LV titres at shake flask volumes (40 mL). Here, the inventors demonstrate that prostratin is effective at increasing titre during the production of an alternative lentivirus used for gene therapy, equine infectious anaemia virus (EIAV).

Materials and Methods

Suspension Cell Culture, Transfection and 3^{rd} Generation, SIN-Lentiviral Vector Production

HEK1.65s cells were grown in serum-free Freestyle (FS) media+0.1% cholesterol lipid concentrate (CLC) (Gibco) at 37° C. in 5% CO₂ in a shaking incubator.

EIAV CMV-GFP vector was produced in 125 mL Erlenmeyer shake flasks under the following conditions: HEK1.65s cells were seeded in 20 mL serum-free media and approximately 24 hours later the cells were transfected with EIAV-GFP-CMV, EIAV Gag-Pol and VSV-G. Transfection was mediated by mixing DNA with Lipofectamine 2000CD in serum-free media as per manufacturer's protocol (Life Technologies). Cells were incubated at 37° C. in 5% CO₂ in a shaking incubator throughout the course of vector production.

Vector production in shake flasks 1˜4 was induced using a final concentration of 10 mM sodium butyrate. At the same time as sodium butyrate induction, prostratin (dissolved in DMSO) was added to shake flasks 3 and 4 to give a final concentration of 11 μM prostratin and 0.2% (v/v) DMSO (Table 10). Vector supernatant was harvested approximately 24 hours after induction and filtered using a 0.45 μm syringe filter.

TABLE 10
Induction compositions of shake flasks.

| Shake Flask | NaBut (mM) | Prostratin (μM) |
|---|---|---|
| 1 | 10 | N/A |
| 2 | 10 | N/A |
| 3 | 10 | 11 |
| 4 | 10 | 11 |

Lentiviral Vector Titration Assay

For lentiviral vector titration by FACS, HEK293T cells were seeded in complete media. Wells were transduced approximately 24 hours after seeding with 500 μL vector diluted in complete media+8 μg/mL polybrene and wells were topped up with 1 mL complete media between 3-6 hours after transduction. The transduced cells were incubated for 3 days at 37° C. in 5% CO₂. Cells were detached using TrypLE (Gibco) and re-suspended in complete media for flow cytometry. Percent GFP expression was measured using Live/Singlet/GFP⁺ gating. Titres were calculated based on percent GFP⁺ cells, a cell count at transduction of 1.85×10⁵, the vector dilution factor and volume vector at transduction.

Results and Discussion

Including prostratin for the induction of EIAV-CMV-GFP vector resulted in a 2-fold increase in titre compared to the control titre (9.1E+05 TU/mL with prostratin at induction vs 4.4E+05 TU/mL without prostratin) (FIG. 17).

### Example 8: The Use of Prostratin to Boost Expression from a Variety of Constitutive Promoters to Model Induction of Expression of Viral Vector Component Expression During Vector Production

A variety of constitutive promoters were cloned into a GFP-expression plasmid: Cytomegalovirus promoter—CMV; Rous Sarcoma virus U3 promoter—RSV; CAG synthetic promoter (CMV enhancer, promoter-exon/intron of chicken beta-actin gene, the splice acceptor of the rabbit beta-globin gene); Chinese hamster EF-1alpha-1 promoter—CHEF1; GRP78/BiP (stress-inducible) promoter—GRP78; Ubiquitin-C promoter—UBC; HIV-1 U3 promoter—HIV-1 U3; Human ferritin heavy chain promoter—FERH; and Simian virus 40 promoter—SV40.

To model expression of a viral vector component during vector production (e.g. AAV capsid, LV genome, etc), suspension (serum-free) HEK293T cells were transfected separately at two input amounts with each pPromoter-GFP DNA (to model expression at alternative ratios). All cultures were treated with sodium butyrate (10 mM; a typical induction treatment) post-transfection, simultaneously with or without 11 μM prostratin. Approximately two days post-transfection, cultures were analysed by flow cytometry to assess transfection efficiency and GFP expression levels. Transgene expression scores were generated for each culture/condition by multiplying % GFP positive cells with the median fluorescence intensity (MFI) values (arbitrary units). These data are plotted in FIG. 18, and show induction of expression from the promoters, surprisingly even in the presence of sodium butyrate which is a well-known inducer of gene expression. This suggests that prostratin is inducing promoter activity in a mechanism distinct from that of sodium butyrate, and hence allowing both compounds to be used simultaneously if required. Of note is the clear induction of three powerful promoters—CMV, CAG and RSV—at both pDNA input levels. This demonstrates the utility for use of prostratin to induce greater gene expression from already strong promoters for use in viral vector component expression systems, such as (and not limited to) LVs, AAVs and AdVs. For example others have shown that AAV rep and cap genes can be individually expressed by heterologous promoters such as CMV and RSV (Vincent et al., 1997; Journal of Virology, pg 1897-1905). Given the observed induction of prostratin on these (and other) promoters herein, it is reasonable to expect prostratin to increase expression of any viral vector packaging component should it be transcriptionally dependent on any of these promoters exemplified here, and any others can be easily tested for their induction by prostratin as demonstrated here.

### REFERENCES

Alotaibi D et al. (2018) Potential anticancer effect of prostratin through SIK3 inhibition. Oncol Lett 15(3): 3252-3258
Beans E J et al. (2013) Highly potent, synthetically accessible prostratin analogs induce latent HIV expression in vitro and ex vivo. PNAS 110(29): 11698-11703
Behrens R T et al. (2017) Nuclear Export Signal Masking Regulates HIV-1 Rev Trafficking and Viral RNA Nuclear Export. J Virol 91(3): e02107-02116.
Chen D, Wang H, Aweya J J et al. (2016) HMBA Enhances Prostratin-Induced Activation of Latent HIV-1 via Suppressing the Expression of Negative Feedback Regulator A20/TNFAIP3 in NF-kB Signalling. Biomed Res Int 5173205
Cooper J et al. (2011) Filamin A protein interacts with human immunodeficiency virus type 1 Gag protein and contributes to productive particle assembly. J Biol Chem 286(32):28498-28510
Dotson D et al. (2016) Filamin A Is Involved in HIV-1 Vpu-mediated Evasion of Host Restriction by Modulating Tetherin Expression. J Biol Chem 291(8):4236-4246
Hongpaisan J & Alkon D L (2007) A structural basis for enhancement of long-term associative memory in single dendritic spines regulated by PKC. PNAS. 104(49):19571-19576
Kulkosky J et al. (2001) Prostratin: activation of latent HIV-1 expression suggests a potential inductive adjuvant therapy for HAART. Blood 98(10):3006-3015
Marsden M D, Wu X, Navab S M et al. (2018) Characterisation of design, synthetically accessible bryostatin analog HIV latency reversing agent. Virology 520: 83-93
Newton A C (2010) Protein kinase C: poised to signal. Am J Physiol Endocrinol Metab 298(3): E395-E402.
Reuse S et al. (2009) Synergistic Activation of HIV-1 Expression by Deacetylase Inhibitors and Prostratin: Implications for Treatment of Latent Infection. PLoS One 4(6): e6093
Williams S A, Chen L-F, Kwon H et al. (2004) Prostratin Antagonizes HIV latency by Activating NF-kB. JBC 279(40): 42008-42017

1. A method for producing a viral vector, the method comprising culturing a cell comprising nucleic acid sequences encoding viral vector components in a cell culture medium that comprises a PKC activator.

2. The method of claim 1, wherein the viral vector is a self-inactivating viral vector.

3. The method of any preceding claim, wherein the PKC activator is prostratin or phorbol 12-myristate 13-acetate, an analogue, derivative or pharmaceutically acceptable salt thereof.

4. The method of claim 3, wherein:
a) prostratin is in the cell culture medium at a concentration of at least about 0.5 μM, optionally wherein prostratin is at a concentration of from about 0.5 to about 32 μM; or
b) phorbol 12-myristate 13-acetate is in the cell culture medium at a concentration of at least about 1 nM, optionally wherein phorbol 12-myristate 13-acetate is at a concentration of from about 1 to about 32 nM.

5. The method of any preceding claim, wherein the viral vector is a lentiviral vector and a modified U1 snRNA is co-expressed with the lentiviral vector components, wherein said modified U1 snRNA binds to a nucleotide sequence within the packaging region of the lentiviral vector genome sequence.

6. The method of any preceding claim, wherein the viral vector is a lentiviral vector and wherein splicing activity from the major splice donor region of the lentiviral vector genome has been functionally ablated.

7. The method of any preceding claim, wherein the viral vector is a lentiviral vector, wherein the lentiviral vector genome has been mutated in the major splice donor region or mutated in the major splice donor region and at least one cryptic splice donor region.

8. The method of any preceding claim, wherein the cell culture medium further comprises a HDAC inhibitor.

9. The method of claim 8, wherein the HDAC inhibitor is an aliphatic HDAC inhibitor or a hydroxamic acid HDAC inhibitor.

10. The method of claim 9, wherein the aliphatic HDAC inhibitor is sodium butyrate, sodium valproate or valeric acid, an analogue, derivative or pharmaceutically acceptable salt thereof.

11. The method of any of claims 8 to 10, wherein the PKC activator is prostratin and the HDAC inhibitor is sodium butyrate.

12. The method of claim 9, wherein the hydroxamic acid HDAC inhibitor is suberanilohydroxamic acid, an analogue, derivative or pharmaceutically acceptable salt thereof.

13. The method of any of claims 10 to 12, wherein:
a) sodium butyrate is in the cell culture medium at a concentration of at least about 2.5 mM, optionally wherein sodium butyrate is at a concentration of from about 2.5 to about 30 mM;
b) sodium valproate is in the cell culture medium at a concentration of at least about 3 mM, optionally wherein sodium valproate is at a concentration of from about 3 to about 30 mM;
c) valeric acid is in the cell culture medium at a concentration of at least about 3 mM, optionally wherein valeric acid is at a concentration of from about 3 to about 30 mM; or
d) suberanilohydroxamic acid is in the cell culture medium at a concentration of at least about 0.5 μM, optionally wherein suberanilohydroxamic acid is at a concentration of from about 0.5 to about 16 μM.

14. The method of any preceding claim, wherein the cell is a transiently transfected production cell.

15. The method of any of claims 1 to 13, wherein the cell is a stable producer cell.

16. The method of any preceding claim, wherein the cell is a eukaryotic cell.

17. The method of claim 16, wherein the cell is a mammalian cell.

18. The method of claim 17, wherein the cell is a human cell.

19. The method of any preceding claim, wherein the cell is adherent.

20. The method of any preceding claim, wherein the cell is a HEK293 cell, or a derivative thereof.

21. The method of claim 20, wherein the HEK293 production cell is a HEK293T cell.

22. The method of any of claims 1 to 18, wherein the cell is in suspension.

23. The method of any preceding claim, wherein the viral vector is selected from the group consisting of: a retroviral vector, an adenoviral vector, an adeno-associated viral vector, a herpes simplex viral vector and a vaccinia viral vector.

24. The method of claim 23, wherein the retroviral vector is a lentiviral vector.

25. The method of claim 24, wherein the lentiviral vector is selected from the group consisting of: HIV-1, HIV-2, SIV, FIV, BIV, EIAV, CAEV and visna lentiviral vector.

26. The method of any of preceding claim, wherein the viral vector comprises a nucleotide of interest (NOI).

27. The method of any preceding claim, wherein the cell culture medium comprises a volume of at least about 5 litres of medium.

28. The method of any preceding claim, wherein the cell culture medium is serum-free.

29. The method of any preceding claim, wherein at least one nucleic acid sequence encoding a viral vector component is operably linked to a promoter selected from the group consisting of: a CMV promoter, an RSV promoter, a CAG synthetic promoter, a CHEF1 promoter, a GRP78 promoter, a UBC promoter, an HIV-1 U3 promoter, and a FERH promoter.

30. The method of claim 30, wherein the promoter is selected from the group consisting of: a CMV promoter, an RSV promoter, and a CAG synthetic promoter.

31. A viral vector production system comprising:
i) a cell comprising nucleic acid sequences encoding viral vector components; and
ii) a cell culture medium that comprises a PKC activator.

32. The viral vector production system of claim 31, wherein the viral vector is a self-inactivating viral vector.

33. The viral vector production system of claim 31 or 32, wherein the PKC activator is prostratin or phorbol 12-myristate 13-acetate, an analogue, derivative or pharmaceutically acceptable salt thereof.

34. The viral vector production system of claim 33, wherein:
a) prostratin is in the cell culture medium at a concentration of at least about 0.5 μM, optionally wherein prostratin is at a concentration of from about 0.5 to about 32 μM; or
b) phorbol 12-myristate 13-acetate is in the cell culture medium at a concentration of at least about 1 nM, optionally wherein phorbol 12-myristate 13-acetate is at a concentration of from about 1 to about 32 nM.

35. The viral vector production system of any of claims 31 to 34, further comprising a nucleic acid sequence encoding a modified U1 snRNA, wherein the modified U1 snRNA binds to a nucleotide sequence within the packaging region of the lentiviral vector genome sequence.

36. The viral vector production system of any of claims 31 to 35, wherein the viral vector is a lentiviral vector and wherein splicing activity from the major splice donor region of the lentiviral vector genome has been functionally ablated.

37. The viral vector production system of any of claims 31 to 36, wherein the viral vector is a lentiviral vector and wherein the lentiviral vector genome has been mutated in the major splice donor region or mutated in the major splice donor region and at least one cryptic splice donor region.

38. The viral vector production system of any of claims 31 to 37, wherein the cell culture medium further comprises a HDAC inhibitor.

39. The viral vector production system of claim 38, wherein the HDAC inhibitor is an aliphatic HDAC inhibitor or a hydroxamic acid HDAC inhibitor.

40. The viral vector production system of claim 39, wherein the aliphatic HDAC inhibitor is sodium butyrate, sodium valproate or valeric acid, an analogue, derivative or pharmaceutically acceptable salt thereof.

41. The viral vector production system of any of claims 38 to 40, wherein the PKC activator is prostratin and the HDAC inhibitor is sodium butyrate.

42. The viral vector production system of claim 39, wherein the hydroxamic acid HDAC inhibitor is suberanilohydroxamic acid, an analogue, derivative or pharmaceutically acceptable salt thereof.

43. The viral vector production system of any of claims 40 to 42, wherein:
a) sodium butyrate is in the cell culture medium at a concentration of at least about 2.5 mM, optionally wherein sodium butyrate is at a concentration of from about 2.5 to about 30 mM;
b) sodium valproate is in the cell culture medium at a concentration of at least about 3 mM, optionally wherein sodium valproate is at a concentration of from about 3 to about 30 mM;
c) valeric acid is in the cell culture medium at a concentration of at least about 3 mM, optionally wherein valeric acid is at a concentration of from about 3 to about 30 mM; or
d) suberanilohydroxamic acid is in the cell culture medium at a concentration of at least about 0.5 μM, optionally wherein suberanilohydroxamic acid is at a concentration of from about 0.5 to about 16 μM.

44. The viral vector production system of any of claims 31 to 43, wherein the cell is a transiently transfected production cell.

45. The viral vector production system of any of claims 31 to 43, wherein the cell is a stable producer cell.

46. The viral vector production system of any of claims 31 to 45, wherein the cell is a eukaryotic cell.

47. The viral vector production system of claim 46, wherein the cell is a mammalian cell.

48. The viral vector production system of claim 47, wherein the cell is a human cell.

49. The viral vector production system of any of claims 31 to 48, wherein the cell is adherent.

50. The viral vector production system of any of claims 31 to 49, wherein the cell is a HEK293 cell, or a derivative thereof.

51. The viral vector production system of claim 50, wherein the HEK293 production cell is a HEK293T cell.

52. The viral vector production system of any of claims 31 to 48, wherein the cell is in suspension.

53. The viral vector production system of any of claims 31 to 52, wherein the viral vector is selected from the group consisting of: a retroviral vector, an adenoviral vector, an adeno-associated viral vector, a herpes simplex viral vector and a vaccinia viral vector.

54. The viral vector production system of claim 53, wherein the retroviral vector is a lentiviral vector.

55. The viral vector production system of claim 54, wherein the lentiviral vector is selected from the group consisting of: HIV-1, HIV-2, SIV, FIV, BIV, EIAV, CAEV and visna lentiviral vector.

56. The viral vector production system of any of claims 31 to 55, wherein the viral vector comprises a nucleotide of interest (NOI).

57. The viral vector production system of any of claims 31 to 56, wherein at least one nucleic acid sequence encoding a viral vector component is operably linked to a promoter selected from the group consisting of: a CMV promoter, an RSV promoter, a CAG synthetic promoter, a CHEF1 promoter, a GRP78 promoter, a UBC promoter, an HIV-1 U3 promoter, and a FERH promoter.

58. The viral vector production system of claim 57, wherein the promoter is selected from the group consisting of: a CMV promoter, an RSV promoter, and a CAG synthetic promoter

59. The viral vector production system of any of claims 31 to 58, wherein the cell culture medium is serum-free.

60. Use of a PKC activator for increasing viral vector titre during viral vector production.

61. The use according to claim 60, wherein the PKC activator is used in combination with a HDAC inhibitor.

62. The use according to claim 60 or 61, wherein the viral vector is a self-inactivating viral vector.

63. The use according to claims 60 to 62, wherein the PKC activator is prostratin or phorbol 12-myristate 13-acetate, an analogue, derivative or pharmaceutically acceptable salt thereof.

64. The use according to claims 61 to 63, wherein the HDAC inhibitor is an aliphatic HDAC inhibitor or a hydroxamic acid HDAC inhibitor.

65. The use according to claim 64, wherein the aliphatic HDAC inhibitor is sodium butyrate, sodium valproate or valeric acid, an analogue, derivative or pharmaceutically acceptable salt thereof.

66. The use according to claims 61 to 65, wherein the PKC activator is prostratin and the HDAC inhibitor is sodium butyrate.

67. The use according to claim 64, wherein the hydroxamic acid HDAC inhibitor is suberanilohydroxamic acid, an analogue, derivative or pharmaceutically acceptable salt thereof.

68. The use according to any of claims 60 to 67, wherein the viral vector is produced from a cell comprising nucleic acid sequences encoding viral vector components, wherein at least one of the nucleic acid sequences is operably linked to a promoter selected from the group consisting of: a CMV promoter, an RSV promoter, a CAG synthetic promoter, a CHEF1 promoter, a GRP78 promoter, a UBC promoter, an HIV-1 U3 promoter, and a FERH promoter.

69. The use according to claim 68, wherein the promoter is selected from the group consisting of: a CMV promoter, an RSV promoter, and a CAG synthetic promoter.
